(19) United States
(12) Patent Application Publication
(10) Pub. No.: US 2020/0078335 A1
(43) Pub. Date: Mar. 12, 2020

(54) COMPOSITIONS AND METHODS TO REDUCE PATHOGENESIS

(71) Applicant: The Board Of Supervisors Of Louisiana State University And Agricultural And Mechanical College, Baton Rouge, LA (US)

(72) Inventors: Timothy Paul Foster, Slidell, LA (US); Charles David Nichols, New Orleans, LA (US)

(21) Appl. No.: 16/610,322

(22) PCT Filed: May 1, 2018
(86) PCT No.: PCT/US2018/030443

§ 371 Date: Nov. 1, 2019

Related U.S. Application Data
(60) Provisional application No. 62/492,835, filed on May 1, 2017.
(60) Provisional application No. 62/492,841, filed on May 1, 2017.

(51) Int. Cl. A61K 31/4045; A61K 31/137; A61K 31/48; A61K 45/06; A61K 9/00; A61P 27/02; A61P 31/22

(52) U.S. Cl. CPC A61K 31/4045; A61K 31/137; A61K 31/48; A61P 31/22; A61K 9/0048; A61P 27/02; A61K 45/06

(57) ABSTRACT

This invention is directed to compositions, methods and kits for ameliorating or reducing pathogenesis of a disease by administering to a subject a serotonin receptor agonist.

This application claims priority from U.S. Provisional Application No. 62/492,835, filed on May 1, 2017, and from U.S. Provisional Application No. 62/492,841, filed on May 1, 2017, the entire contents of each which are incorporated herein by reference in their entireties.

### GOVERNMENT INTERESTS

This invention was made with government support under Grant No. P30GM106392 awarded by the National Institutes of Health and Project No. 08-69-04921 awarded by the US Department of Commerce Economic Development Administration. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to compositions, methods, and kits for ameliorating or reducing pathogenesis of an ocular disease by administering to a subject a serotonin receptor agonist.

### BACKGROUND OF THE INVENTION

Serotonin or 5-hydroxytryptamine (5-HT) is a small monoamine molecule primarily known for its role as a neurotransmitter. Within the brain, 5-HT modulates a variety of behaviors including cognition, mood, aggression, mating, feeding, and sleep (Nichols and Nichols, 2008). These behaviors are mediated through interactions at seven different receptor families (5-HT₁₋₇) comprised of fourteen distinct subtypes (Nichols and Nichols, 2008). Each of these are G-protein coupled receptors, with the exception of the 5-HT₃ receptor, which is a ligand-gated ion channel. Of all the serotonin receptors, the 5-HT_{2A} receptor, which is known to primarily couple to the Gαq effector pathway (Roth et al., 1986), has been the one most closely linked to complex behaviors.

### SUMMARY OF THE INVENTION

Embodiments as described herein are directed towards a method of reducing or ameliorating vascularization and vascularization-associated disease in a non-ocular tissue of a subject. In embodiments, the method comprises administering to the subject suffering from a disease associated with vascularization or a vascularization-associated pathology within a tissue a therapeutically effective amount of a composition comprising a serotonin receptor agonist, wherein the tissue is not an ocular tissue.

Embodiments as described herein are directed towards a method of reducing or ameliorating symptoms associated with vascularization or vascularization-associated pathologies in a non-ocular tissue of a subject. In embodiments, the method comprises administering to the subject suffering from a disease associated with neovascularization in a tissue a therapeutically effective amount of a composition comprising a serotonin receptor agonist, wherein the serotonin receptor agonist reduces or ameliorates symptoms associated with dysregulation of vasculature, wherein the tissue is not an ocular tissue.

A method of reducing or ameliorating vascularization-associated disease pathology in an ocular tissue of a subject comprising administering to the subject afflicted with a disease associated with a vascularization-associated pathology a therapeutically effective amount of a composition comprising a serotonin receptor agonist.

In embodiments, vascularization-associated pathologies comprise neovascularization; angiogenesis, for example that of blood vessels or that of lymphatics; vasoconstriction or vasodialation, for example that of blood vessels or that of lymphatics; vascular leakage, vascular permeability, edema, hypertension; ischemia; vascular occlusons; haemmoraghing, and increased hypersensitivity reactions or disorders.

Embodiments as described herein are directed towards a method of reducing or ameliorating a hypersensitivitiy-associated disease process in an immunologically-restricted tissue of a subject. In embodiments, the method comprises administering to a subject afflicted with a hypersensitivity-associated disease process in an immunologically-restricted tissue a therapeutically effective amount of a composition comprising a serotonin receptor agonist, wherein the serotonin receptor agonist reduces the hypersensitivity-associated disease process in the immunologically-restricted tissue of the subject.

In embodiments, the tissue comprises an immunologically-restricted tissue. Non-limiting examples of immunologically-restricted tissues comprise tissues of the lung, skin, brain, eyes, gut or combination thereof.

Embodiments as described herein comprise a method of reducing or ameliorating hypersensitivity in an immunologically-restricted tissue of a subject. In embodiments, the method comprises administering to a subject suffering from hypersensitivity in an immunologically-restricted tissue a therapeutically effective amount of a composition comprising a serotonin receptor agonist.

Embodiments as described herein are directed towards a method of treating a vascularization-associated non-ocular disease in a subject. In embodiments, the method comprises administering to a subject afflicted with a vascularization-associated disease a therapeutically effective amount of a composition comprising a serotonin receptor agonist, wherein the serotonin receptor agonist treats the neovascularization-associated disease in the subject, and wherein the disease is not an ocular disease.

Embodiments as described herein are directed towards a method of reducing or ameliorating pathogenesis associated with an infection in a subject. In embodiments, the method comprises administering to a subject suffering from at least one pathogenesis associated with an infection a therapeutically effective amount of a composition comprising a serotonin receptor agonist.

Embodiments as described herein are directed towards a method of reducing or ameliorating symptoms associated with a pathogenic infection in a subject. In embodiments, the method comprises administering to a subject afflicted with a pathogenic infection a therapeutically effective amount of a composition comprising a serotonin receptor agonist, wherein the serotonin receptor agonist reduces or ameliorates symptoms associated with the pathogenic infection.

In embodiments, the infection is not an ocular infection, but is a non-ocular infection. In embodiments, an immunologically-restricted tissue is infected. For example, a lung tissue is infected, such as with influenza. In embodiments, the infection comprises a resolved infection.

In embodiments, the infection causes pathogenesis in at least one tissue of the subject, non-limiting examples of which comprise angiogenesis, neovascularization, haemoraghing, hypersensitivity, vascular leakage, vascular permeability, hypertension, edema, lymphangiogenesis, or a combination thereof. In embodiments, the pathogenesis affects a tissue of the eye, lung, skin, brain, gut or a combination thereof.

The invention further provides a method of reducing or ameliorating pathogenesis, for example vascularization-associated pathogenesis, in an ocular tissue of a subject, the method comprising administering to a subject suffering from pathogenesis of an ocular tissue a therapeutically effective amount of a composition comprising a serotonin receptor agonist. Non-limiting examples of such ocular tissues comprise aqueous humor, choroid, conjunctiva, cornea, iris ciliary body, lens, optic nerve, optic nerve head, retina, sclera, various glands, virteous humor, or a combination thereof.

In embodiments, the ocular tissue comprises a tissue that is normally avascular. A non-limiting example of an avascular tissue of the eye comprises the cornea, including the corneal stroma.

In embodiments, the ocular tissue comprises a tissue that is normally vascularized. In such embodiments, the neovascularization of the ocular tissue can be tightly regulated. In embodiments, the vascularization comprises angiogenesis of lymphatics, angiongenesis of blood vessels, or a combination thereof.

In embodiments, the ocular tissue can be in the anterior segment of the eye, such as the tissues located between the front surface of the cornea and the vitreous. In other embodiments, the ocular tissue can be in the posterior segment of the eye, such as the vitreous, retina, optic disc, choroid, and pars plana. In embodiments, pathogenesis comprises a chronic condition.

Non-limiting examples of pathogenesis of the eye comprise hypersensitivity, vascularization, angiogenesis, lymphangiogenesis, edema, corneal epithelial defects, fibrosis, haemoraghing, ischemia, increased intraocular pressure, increased oxygen saturation, reduced vision, or a combination thereof.

Non-limiting examples of pathogenesis of the lung comprise neovascularization, inflammation, vascular leakage, vascular permeability, hypersensitivities, angiogenesis, fibrosis, decreased oxygen saturaton, decreased lung function, increased cellularity, asthma or a combination thereof.

Non-limiting examples of pathogenesis of the skin comprise neovascularization, hypersensitivities, vascular leakage, vascular permeability, haemorghing, ulceration, dermal hyperproliferation, angiogenesis, fibrosis, or a combination thereof.

Non-limiting examples of pathogenesis of the brain comprise demylenation, neural inflammation, encephalitis, meningitis, viral reactivation from latent neurons, or a combination thereof.

Embodiments as described herein can delay or prevent viral reactivation, shedding transmission, or a combination thereof. For example, embodiments can delay or prevent reactivation from latency, shedding, and/or transmission of HSV from infected individuals, such as those infected with HSV-1 or HSV-2. In embodiments, such delay or prevention of reactivation can delay or prevent the development of secondary diseases and/or pathogenesis, such as ocular diseases, including those described herein.

In embodiments, a 5HT agonist, for example DOI, can delay or inhibit neuronal reactivation of a virus, such as HSV-1, from latency, such as within the trigeminal ganglia.

Embodiments as described herein can maintain viral latency, so as to prevent viral reactivation.

Embodiments can prevent the progression of acute pathogenesis to chronic pathogenesis.

In embodiments, the infection comprises a viral infection, a bacterial infection, a fungal infection, a protozoan infection, or a combination thereof.

In embodiments, a double-stranded DNA virus causes infection, non-limiting examples of which comprise an adenovirus, herpes virus, John Cunningham virus, Cytomegalovirus, Parvovirus, human papilloma virus, polyoma virus, poxvirus or varicella zoster virus. Non-limiting examples of a herpes virus comprises Herpes simplex virus type 1, Herpes simplex virus type 2, or a combination thereof.

In embodiments, a single-stranded RNA virus causes infection, non-limiting examples of which comprise Picomaviridae, Togaviridae, Flaviviridae, Retroviridae, Coronaviridae, paramyxviridae, rhadboviridae, orthomyxoviridae, filoviridae, Arenaviridae, influenza virus, respiratory syncytial virus. SARS coronavirus, rabies virus, measles virus. polio virus, enterovirus, mumps virus, Rubella, Coxackie virus, Parainfluenza, West-Nile Virus, Equine Encephalitis, Picomaviruses, Rhinoviruses, Rabies, Ebola, lymphocytic choriomeningitis virus, or HIV. or a combination thereof.

In embodiments, the viral infection is caused by at least one of a herpes virus, an adenovirus, a respiratory syncytial virus (RSV), and an influenza virus. Non-limiting examples of viral infections comprise viral pneumonia, viral bronchitis, herpetic keratitis, stromal keratitis, adenoviral conjunctivitis, SARS, Acute Respiratory Distress Syndrome, viral meningitis, viral encephalitis, neural inflammation, demyelination, dermatitis, ulceration, virus-induced asthma, virus-induced pulmonary dysfunction or a combination thereof.

In embodiments, serotonin receptor agonists can prevent or lessen viral reactivation from latency within neurons, subsequent viral shedding and transmission from neurons, and recurrent neurological, dermal or ocular disease.

In embodiments, the bacterium that causes infection comprises listeria, a species of Staphylococcus, a species of Legionella, a species of Streptococcus, a species of Pseudomonas, or a combination thereof, non-limiting examples of which comprise at least one of bacterial pneumonia, Legionnaires' disease.

In embodiments, the fungus that causes infection comprises Candida albicans, Aspergillus, Fusarium, non-limiting examples of which comprise pneumonia, candidiasis, keratitis.

In embodiments, the protozoan that causes infection comprises plasmodium, trypanosome, amoeba, giardia, a species of Acanthamoeba, non-limiting examples of which comprise at least one of Acanthamoeba keratitis, malaria, protozoal pneumonia.

In embodiments, the disease comprises a chronic disease.

In embodiments, the disease comprises a disease of the lung, eye, skin, bones and joints, intestines, neuronal system or a combination thereof.

Non-limiting examples of an ocular disease comprises AMD, choroidal vascularization, diabetic retinopathies, ocular hypertension, corneal allograft rejection, allergic reactions, corneal vascularization, dry eye, or glaucoma.

Non-limiting examples of a lung disease comprise pulmonary hypertension, pneumonia, bronchitis, asthma, or nasal polyps. In embodiments, the lung disease is not asthma.

Non-limiting examples of a skin disease comprises psoriasis, warts, allergic dermatitis, contact dermatitis, or Kaposi Sarcoma.

Non-limiting examples of a disease of the bones and joints comprise arthritis.

In embodiments, the intestinal disease is not irritable bowel syndrome or Crohn's disease.

Non-limiting examples of a disease of the neuronal system comprises Alzheimer's disease or virus-mediated neuropathies.

In embodiments, neovascularization comprises angiogenesis of blood vessels, angiogenesis of lymphatic vessels, or a combination thereof.

Embodiments as described herein are directed towards a method of treating a hypersensitivity-associated ocular disease in a subject. In embodiments, the method comprises administering to a subject afflicted with a hypersensitivity-associated ocular disease a therapeutically effective amount of a composition comprising a serotonin receptor agonist, wherein the serotonin receptor agonist treats hypersensitivity-associated ocular disease in the subject.

Embodiments as described herein are directed towards a method of treating a hypersensitivity-associated non-ocular disease in a subject. In embodiments, the method comprises administering to a subject afflicted with a hypersensitivity-associated non-ocular disease a therapeutically effective amount of a composition comprising a serotonin receptor agonist, wherein the serotonin receptor agonist treats hypersensitivity-associated non-ocular disease in the subject.

In embodiments, the hypersensitivity is not TNF-α mediated inflammation.

In embodiments, the serotonin receptor agonist comprises a compound of

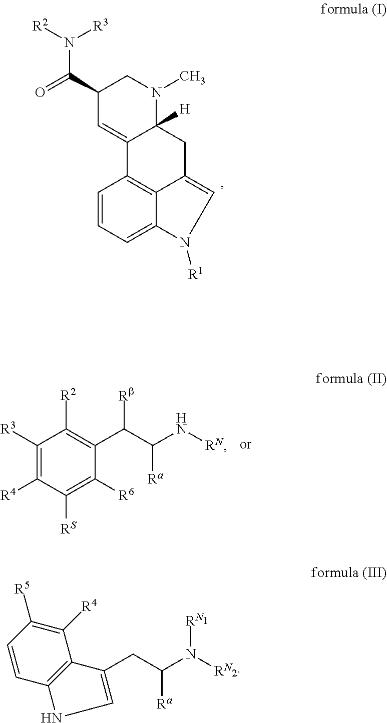

In embodiments, the serotonin receptor agonist comprises 2,5-Dimethoxy-4-iodoamphetamine (DOI).

In other embodiments, the serotonin receptor agonist does not comprise 2,5-Dimethoxy-4-iodoamphetamine (DOI).

Embodiments as described herein can comprise a combination of serotonin receptor agonists.

Embodiments as described herein can further comprise serotonin receptor antagonists. Non-limiting examples of antagonists to 5HT receptors comprise Chlorpromazine, Cyproheptadine, Pizotifen, Oxetorone, Carbinoxamine, Cyproheptadine, Methdilazine, Promethazine, Dolasetron, Granisetron, and Ondansetron.

In embodiments, the serotonin receptor agonist can be administered as a prodrug.

In embodiments, the method comprises a low dose of the serotonin receptor agonist.

Embodiments can further comprises at least one antimicrobial agent, at least one anti-pathogenic agent, at least one drug, or a combination thereof. For example, the antimicrobial agent can comprise an antiviral agent, an antibacterial agent, an antifungal agent, an antiprotozoal agent, or a combination thereof.

Non-limiting examples of an antiviral agent can comprise TFT, Acyclovir, gancyclovir, penciclovir, cidofovir; ribavirin, interferon, phosphonoacetate, Foscarnet, amantadine, Rimatidine, oseltamivir, Valacyclovir, Valgancyclovir, Peramivir, Zanamivir, anti-retroviral drugs.

Non-limiting examples of an antibacterial agent can comprise aminoglycosides, fluoroquinolones, beta-lactams, macrolide, and tetracyclines.

Non-limiting examples of an antifungal agent can comprise at least one polyene, at least one azole, at least one allylamine, echinocardins or a combination thereof.

Non-limiting examples of an antiprotozoal agent comprises chloroquine, pyrimethamine, mefloquine, hydroxychloroquine, metronidazole, atovaquone, or a combination thereof.

In embodiments, administering comprises topical, transdermal, subcutaneous, inhalation, injection, oral, sublingual, or a combination thereof.

In embodiments, the serotonin receptor comprises the 5-HT2A serotonin receptor.

In embodiments, the subject comprises a mammal.

In embodiments, the subject comprises a vertebrate, such as a bird or a mammal. Non-limiting examples of vertebrate mammals comprise a human, dog, cat, rabbit, horse, cow, or pig. Non-limiting examples of a vertebrate bird comprises a chicken, parrot, or hen.

Embodiments are directed towards a composition comprising at least one serotonin receptor agonist and at least one antimicrobial agent. In embodiments, the composition further comprises at least one antipathogenic agent.

In embodiments, the composition comprises a low-dose of the serotonin receptor agonist.

In embodiments, the composition can be administered to a subject as described herein, non-limiting examples of which comprise an ocular drop, dermal patch, ocular gel, topical gel, systemic delivery system, enteric capsule, nebulized inhalant, inhalant, intrathecal, and an injectable.

In embodiments, the antimicrobial inhibits the microbial replicative process.

In embodiments, the composition comprises a compound of

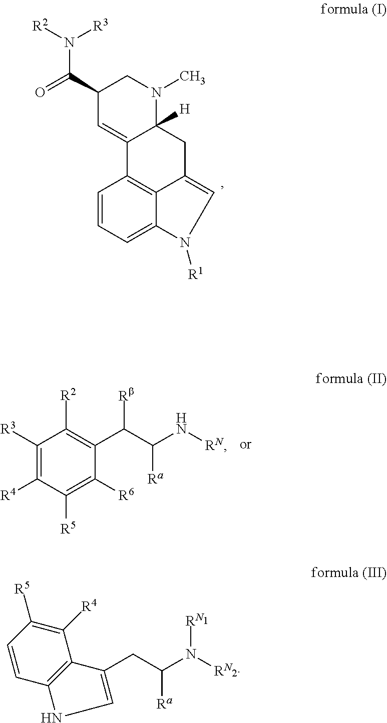

In embodiments, the composition comprises 2,5-Dimethoxy-4-iodoamphetamine (DOI).

In other embodiments, the composition does not comprise 2,5-Dimethoxy-4-iodoamphetamine (DOI).

In embodiments, the serotonin receptor agonist comprises a chemical having the

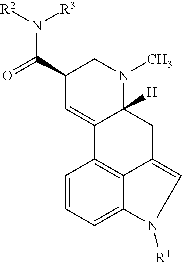

following formula:
wherein R¹, R², and R³ are selected from the group comprising CH₂CH₃, CH(CH₃)CH₂CH₃, CH(CH₃)CH₂CH₂CH₃, C₂H₅, CH₂CH₂CH₃, CH(CH₃)₂ and H.

In embodiments, the serotonin receptor agonist comprises a chemical having the

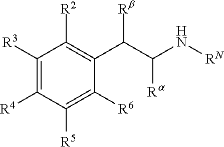

following formula:
wherein R^{α}, R^{β}, R², R³, R⁴, R⁵, R⁶ and R^{N} are selected from the group comprising OCH₃, CH₃, SCH₃, Br, I, CH₂CH(CH₃)₂, and H.

In embodiments, the serotonin receptor agonist comprises a chemical having the following formula:

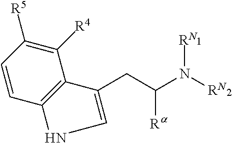

wherein R^{α}, R^{N}₁, R^{N}₂, R⁴ and R⁵ are selected from the group comprising C, CH₃, OH, F, OCH₃ and H.

### BRIEF DESCRIPTION OF THE FIGURES

FIG. 1 shows the comparison of acute and chronic disease scores in C57Black mice following treatment with BS, XTPFDOI or 0.5% TFT+XTPFDOI. Treatment: 4 ul/eye/4× daily ONLY FOR 8 days; Infection model Herpes Stromal Keratitis; C57Black; HSV-1 RE; 12,000 PFU/eye; Clinical Assessment Parameters Shown: Slit-Lamp Biomicroscopy of Eye; Stromal Opacity/Inflammation; Corneal Neovascularization

FIG. 2 shows comparison of Acute and Chronic Disease Scores in BALBc mice following treatment with BSS, 1% TFT, and XTPFDOI; Treatment: 4 ul/eye/4× daily ONLY FOR 8 days; Infection model Herpes Stromal Keratitis; BALBc; HSV-1 RE; 10,000 PFU/eye; Clinical Assessment Parameters Shown: Weight; Slit-Lamp Biomicroscopy of Eye; Stromal Opacity/Inflammation; Corneal Neovascularization.

FIG. 3 shows ability of DOI to control long-term chronic effects of HSV-mediated stromal keratitis: DAY 15 post infection. The three eyes in this group that had not clinically resolved disease still had low clinical scores associated with their pathology as shown in the accompanying pathology.

FIG. 4 shows ocular histology of eyes from BalBc experiments examining ability of DOI to control long-term chronic effects of HSV-mediated stromal keratitis: DAY 15 post infection. Uninfected normal eyes.

FIG. 5A, FIG. 5B, and FIG. 5C show ocular histology of eyes from BalBc experiments examining ability of DOI to control long-term chronic effects of HSV-mediated stromal keratitis: DAY 15 post infection. HSV/RE Infected; Control BSS Treatment Drops; FIG. 5A shows eye 1, FIG. 5B shows eye 2, FIG. 5C shows eye 3.

FIG. 6A, FIG. 6B, FIG. 6C show ocular histology of eyes from BalBc experiments examining ability of DOI to control long-term chronic effects of HSV-mediated stromal keratitis: DAY 15 post infection. HSV/RE Infected; Control 1% TFT Antiviral Treatment Drops: FIG. 6A shows eye 1, FIG. 6B shows eye 1, FIG. 6C shows eye 2 (worse of the Tx group).

FIG. 7 shows comparative preclinical assessment of therapeutic efficacy of a 5-HT receptor agonist (XTPFDOI, red), compared to the gold standard ocular antiviral 1% TFT/Viroptic (blue) or control saline drops (black) in a herpetic stromal keratitis ocular chronic disease model. DOI drops were topically applied for 7 days post infection and chronic disease was assessed up to day 15. DOI suppressed development of all clinically scored parameters with 60% of eyes exhibiting complete clinical resolution by day 15.

FIG. 8 shows histopathological analysis of representative eyes from clinical studies described herein. Top panels: The corneas of uninfected mouse eyes Exhibit regular and consistent uninterrupted outermost epithelial barrier, and an underlying tight corneal stromal layer of even thickness. There is a complete absence of inflammatory or red blood cells and no vascularization of corneal tissue. 2nd row panels: HSV infection and long-term inflammatory responses induces disruption of the epithelial layer, thickening of the stroma, and identifiable vascularization of corneal tissue (yellow arrows) with extensive presence of immune infiltrates. 3rd row panels: Despite treatment with the antiviral TFT and complete inhibition of HSV replication, similar disease processes to control Tx predominate at 15d. 4th row panels and enlarged inset: By contrast, eyes treated with the 5-HT agonist DOI have normal ocular morphology with an absence of clinical signs of ocular disease.

FIG. 9 shows histology of a preclinical mouse model of severe pulmonary influenza infection as will be performed for 5-HT agonists according to protocols we have previously established for drug evaluation. Left top panel: Outline of timecourse of clinical illness and treatment parameters of an example drug study. Right top panel: Clinical parameters of disease assessed and scored daily. Mid-panels: comparison of control treated “sick” animal and a test drug that significantly reduced clinical disease Left lower panels: Typical lung pathology associated with this model showing inflammation (A1, red arrows) and congested airways, as well as vascular leakage and bleeding into the lungs (yellow arrows). Right lower panels: Animals treated with this test drug showed mostly clear airways (green arrows) with only localized sites of inflammation and airway occlusion (red arrows). Similar data will be acquired and analyzed in this study with 5-HT agonists

FIG. 10 shows analysis of therapeutic effects of 5-HT receptor modulation on a preclinical mouse model of imiquimod (IMQ)-induced psoriasis. The experimental design is divided into 3 different therapeutic assessments as depicted and explained in the figure.

FIG. 11 shows clinical parameters scored.

FIG. 12 shows experimental protocol.

FIG. 13 shows experimental timeline.

FIG. 14 shows analysis of Mean+/−SEM total reactivated infectious virus per positive trigeminal ganglia (PFU/ml/positive TG).

FIG. 15 shows analysis of Mean+/−SEM total reactivated infectious virus (PFU/ml/TG).

FIG. 16 shows LSD can prevent symptoms of asthma in a rat model, demonstrating the effectiveness of an ergoline class molecule in vivo. Previous work demonstrated effectiveness of DOI (phenethyamine) and psilocin (tryptamine). OVA is ovalbumin, a frequently used allergen that induces robust, allergic pulmonary inflammation in laboratory rodents.

FIG. 17 shows human corneal epithelia cells (HCEC) and immune cells that contribute to HSK development, express 5HT_{2A} receptors. Primary HCEC (top), as well as several primary immune cell types were analyzed by Western blot for 5HT_{2A} receptor expression. 5HT_{2A} receptor expression within the cornea, on innate immune cells, and on activated T cells, which directly contribute to development of HSK, indicates that these cells can respond to 5HT receptor targeting drugs.

FIG. 18 shows (R)-DOI reduces symptoms of herpetic keratitis in a murine model. Antiviral TFT (blue) initially controlled ocular disease development relative to control BSS treated eyes (black). Eight days post infection (DPI), eyes exhibited a markedly increased slit lamp score, corneal opacity, and neovascularization. In contrast, (R)-DOI (500 μM) treated mice (red) exhibited reduced slit lamp scores, corneal opacity and neovascularization compared to control BSS (black) and TFT up to 15 DPI. (R)-DOI treated animals did not experience the severe weight loss that normally accompanies HSV ocular infection.

FIG. 19 shows an HSV-1 infected eye topically treated with (R)-DOI exhibit significantly less signs of immune-mediated herpetic stromal keratitis, including stromal and corneal inflammation, stromal thickening, damaged corneal epithelium, and corneal neovascularization 15 dpi. Clinically representative eyes from HSV-infected mice treated as herein with either Control BSS drops, the anti-herpetic 1% TFT, or (R)-DOI (500 μM) (bottom 3 panels) were histologically examined. Compared to Control BSS and TFT treated eyes, (R)-DOI treated eyes had markedly less disease presentation in all eyes examined.

FIG. 20 shows (R)-DOI inhibits HSV-1 reactivation from latent neurons within the trigeminal ganglia (TG). Reactivation of latent HSV-1 was induced from TG explants from mice previously ocularly infected with HSV-1. Ganglia were either treated with control (Mock treatment; blue) or media that contained 500 nM (R)-DOI (DOI 500 nM; red). The presence of infectious HSV-1 was assessed for 10 consecutive days.

FIG. 21 shows approach to validate embodiments of the invention.

FIG. 22 shows clinical assessments, behavioral assessments, virological assessments, and pathological assessment applicable to embodiments of the invention.

FIG. 23 shows strategy for demonstrating toxicity, safety, and tolerance.

FIG. 24 shows strategy for demonstrating drug delivery, dosing, and distribution.

FIG. 25 shows strategy for demonstrating therapeutic efficacy of 5-HT2a agonists in viral disease models.

FIG. 26 shows strategy for demonstrating anti-neovascularization activity of 5HT2a receptor agonists.

FIG. 27 shows study design and clinical parameters scored.

FIG. 28 shows HSV-1 viral titres

FIG. 29 shows study design and clinical parameters scored

FIG. 30 shows analysis of therapeutic effects of 5-HT receptor modulation on a preclinical mouse model of severe pulmonary influenza infection will be performed according to protocols we have previously established for drug evaluation. Left top panel: Outline of time course of clinical illness and treatment parameters of an example drug study. Right top panel: Clinical parameters of disease assessed and scored daily. Mid-panels: Comparison of control treated “sick” animal and a test drug that significantly reduced clinical disease Left lower panels: Lung pathology associated with this model showing inflammation (A1, red arrows) and congested airways, as well as vascular leakage and bleeding into the lungs (yellow arrows). Right lower panels: Animals treated with this test drug showed mostly clear airways (green arrows) with only localized sites of inflammation and airway occlusion (red arrows). Similar data will be acquired and analyzed in this study.

FIG. 31 shows experimental timeline.

FIG. 32 shows primary human corneal epithelial cells (HCEC), as well as several immune cell types were analyzed by western blot for expression of 5-HT_{2A} receptors, utilizing an antibody against human 5HT_{2A} receptor. Without wishing to be bound by theory, the expression of 5-HT_{2A} receptors within the cornea and on immune cells indicates an ability of these cells to respond to drugs that target this the serotonin-associated pathway. (A) shows the corneal epithelia of human eyes express drug target 5-HT2A receptors. (B) shows activation of T cells induces expression of drug target 5HT2A receptors. Activated CD3+ T cells are responsible for the development of herpetic keratitis. However, circulating T cells are devoid of 5HT2A receptor expression. (C) shows macrophages, contributors to ocular inflammation within diseased eyes, express 5-HT2A receptors constitutively.

FIG. 33 shows the 5HT2A agonist, serotonin, induces vascularization-like replication and tubule growth and in tissue-like spheroids derived from human microvascular endothelial cells (HMEC). In contrast, multiple 5HT2A receptor agonists (DOI, TCB2, and 2CI) unexpectedly abrogated formation of vascular-like tubular growth from HMEC spheroids. HMEC cells were cultured in specially coated U-shaped bottom 96 well plates in order to form tissue-like 3 dimension spheroids. Spheroids were subsequently implanted into wells that contained matrigel basement membranes supplemented with vascular endothelial growth factor (VEGF) or starved (Starvation control-no VEGF). Culture media was treated with either serotonin (50 nM), (R-DOI (100 nM) or TCB2 (500 nM) outgrowth from the spheroids of vascular-like structures was monitored microscopically.

FIG. 34 shows both the 5HT2A agonists (R-DOI & TCB2) and the antagonist (4F4PP) inhibit VEGF-mediated neovascularization from aortic rings (aortic ring neovascularization assay).

FIG. 35 shows both the 5HT2A agonists (R-DOI & TCB2) and the antagonist 4F4PP inhibit VEGF-mediated human vascular endothelium tubule formation (vascularization tubule formation assay).

FIG. 36 shows R-DOI has potential to selectively kill cancerous retinoblastomas. Normal retinal epithelial cells (APRE) or cancerous retinoblastoma cells (Y-79) were treated with the indicated concentrations of R-DOI and monitored for cell-lysis and cytotoxicity at 24 hour intervals up to 72 hours post treatment.

FIG. 37 shows mice treated ocularly with (R)-DOI do not experience significant weight loss associated with HSV-1 infection of the eye. Animals treated with either the antiviral TFT or with control BSS drops experienced weight loss associated with HSV-1 ocular infections that began at day 5 and lasted until 11 days post infection before beginning to rebound. In stark contrast, (R)-DOI treated animals for the most part maintained weight following infection and exhibited a significant difference in weight at day 8 post infection (peak viral-mediated disease is day 7).

FIG. 38 shows comparison of herpetic keratitis-associated disease Ssores in BALBc mice following treatment with BSS, 1% TFT, and (R)-DOI. Although mice treated with the antiviral TFT initially controlled progression of disease, following day 8 post infection, the eyes of these animals exhibited a markedly increased opacity score, indicative of inflammation. By contrast (R)-DOI treated animals increase in stromal opacity was muted compared to BSS controls and antiviral TFT controls.

FIG. 39 shows comparison of herpetic keratitis-associated disease scores in BALBc mice following treatment with BSS, 1% TFT, and (R)-DOI. Mice treated with R-DOI exhibited decreased development of corneal neovascularization, a key contributor to herpetic keratitis development, compared to control BSS treated or antiviral TFT treated eyes.

FIG. 40 shows comparison of herpetickeratitis-associated disease scores in BALBc mice following treatment with BSS, 1% TFT, and (R)-DOI.

FIG. 41 shows ability of DOI to control HSV-mediated stromal keratitis (DAY 15 post infection). * The three eyes in this group that had not clinically resolved disease, still had low clinical scores associated with their pathology as shown in the accompanying pathology.

FIG. 42 shows ability of DOI to control HSV-mediated stromal keratitis. DAY 15 post infection histology of eyes at day 15.

FIG. 43 shows a selective 5-HT2C agonist does not have any efficacy to reduce PenH in the asthma model.

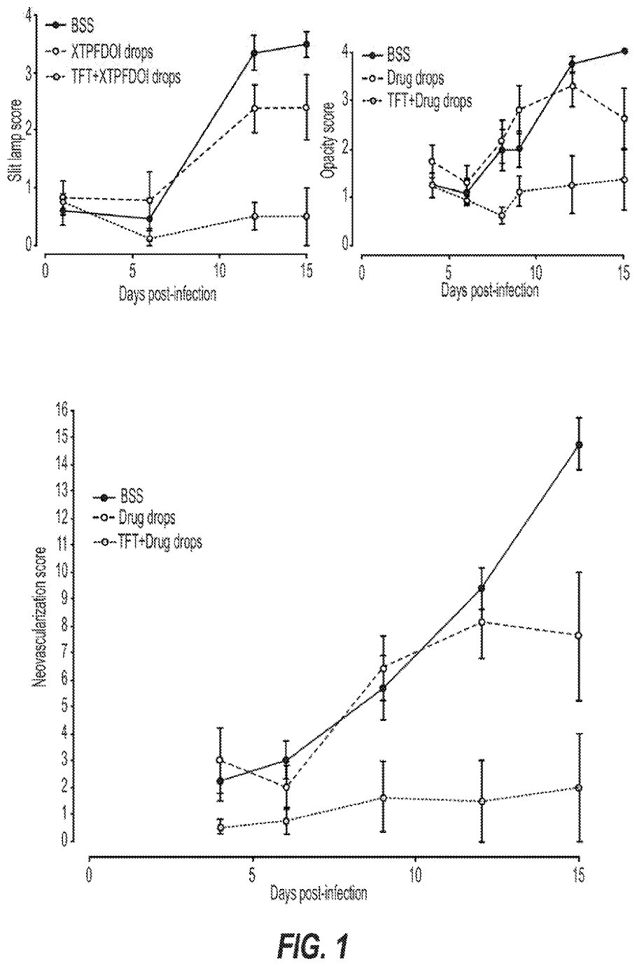

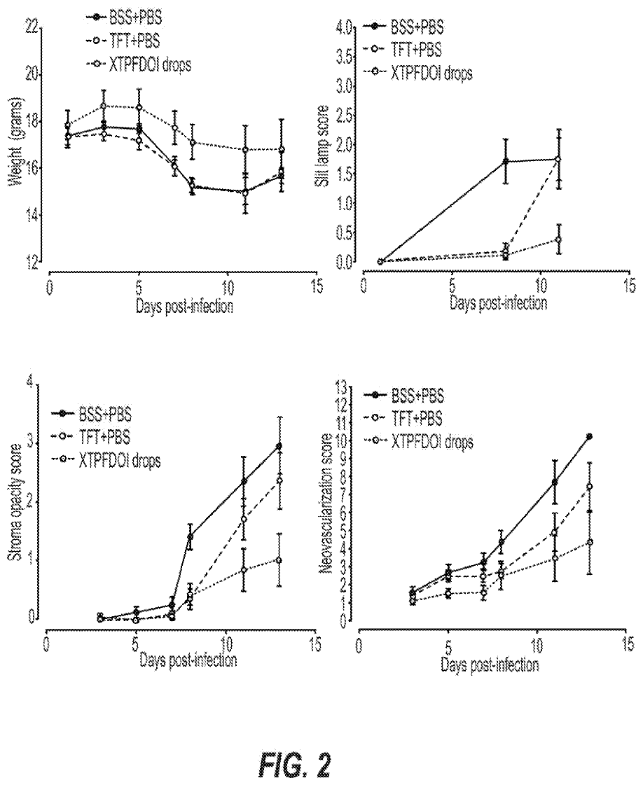

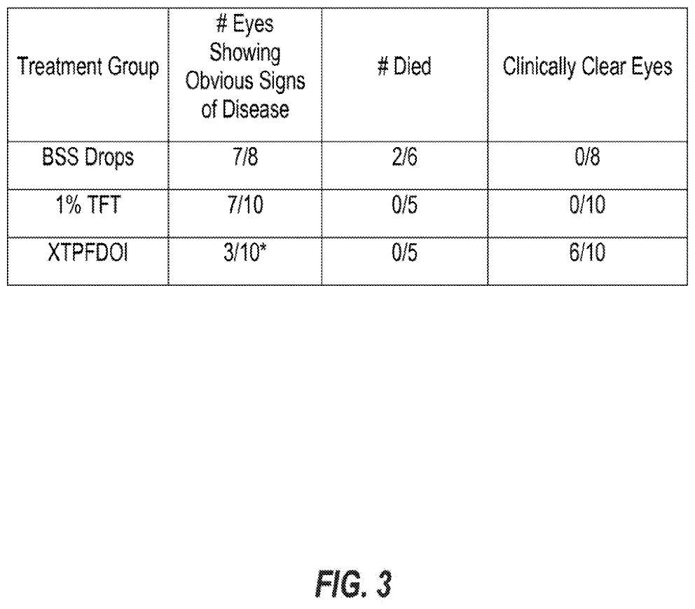

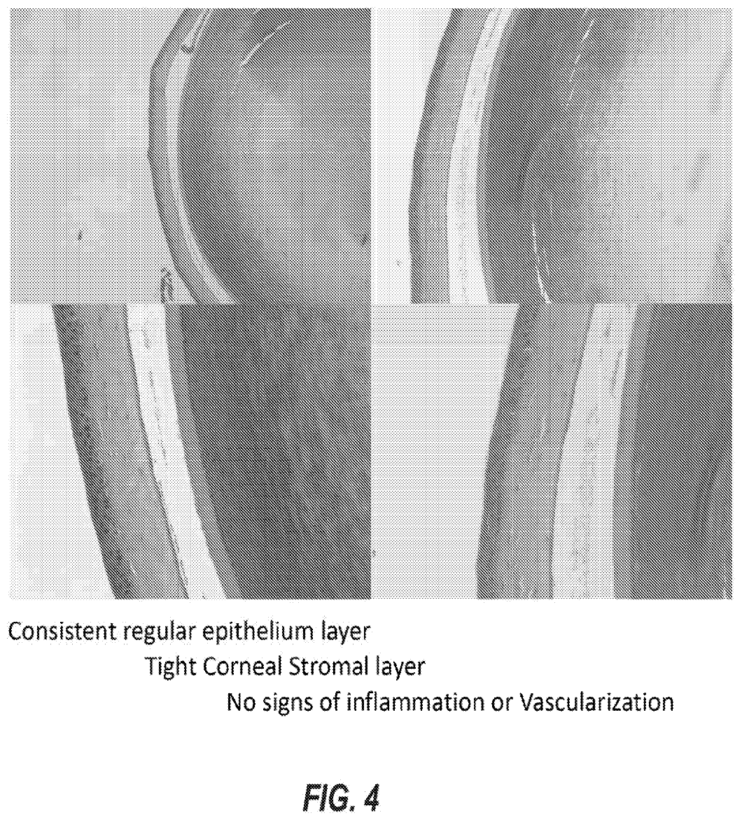

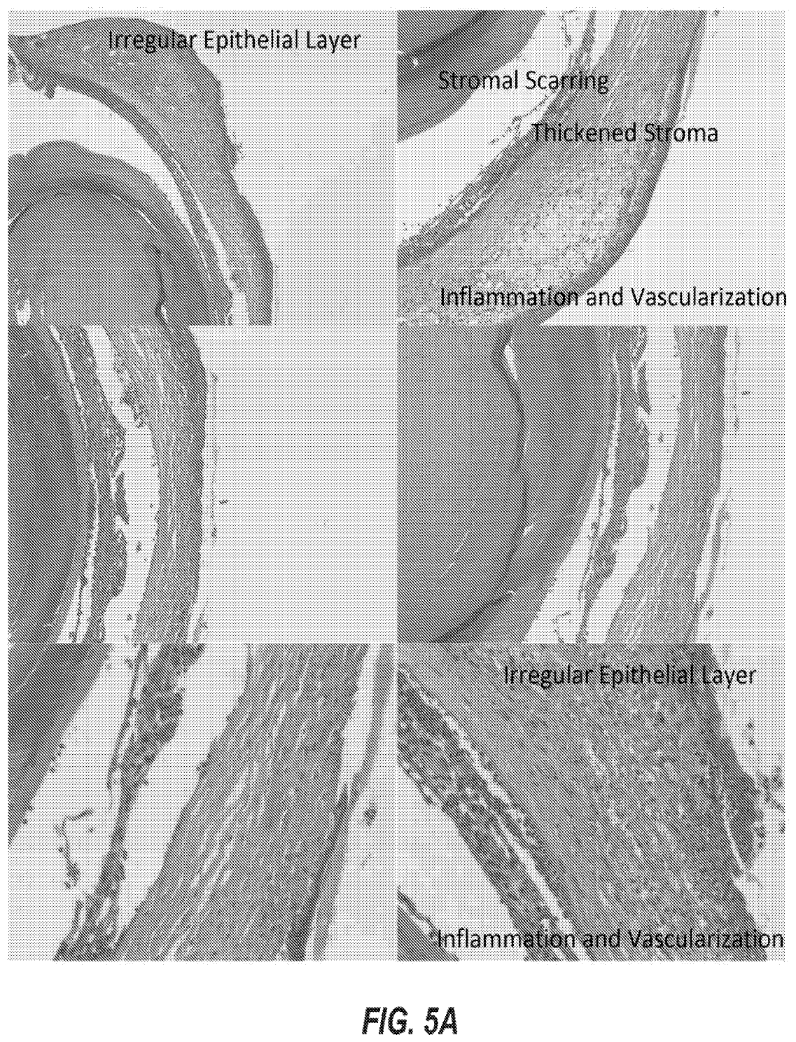

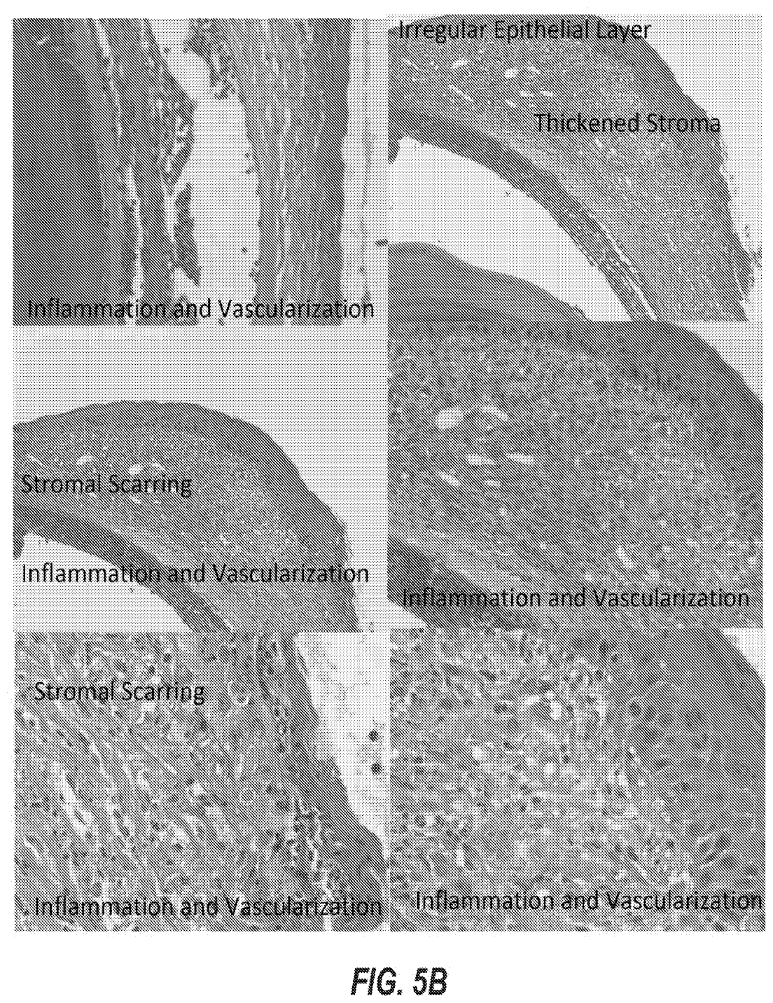

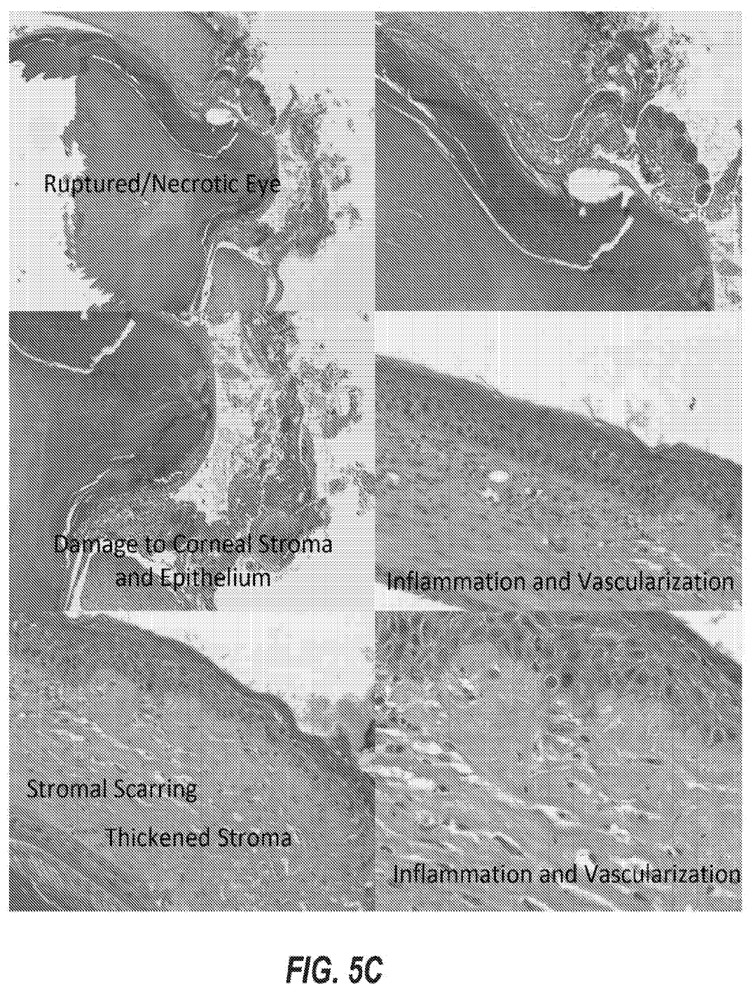

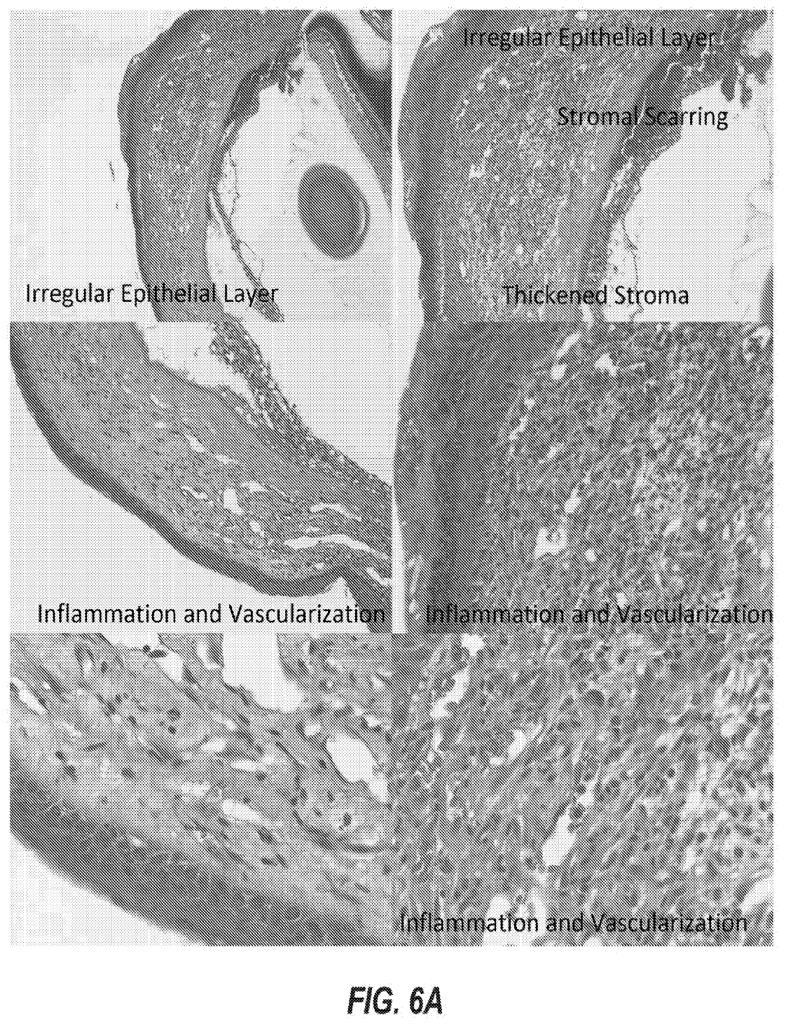

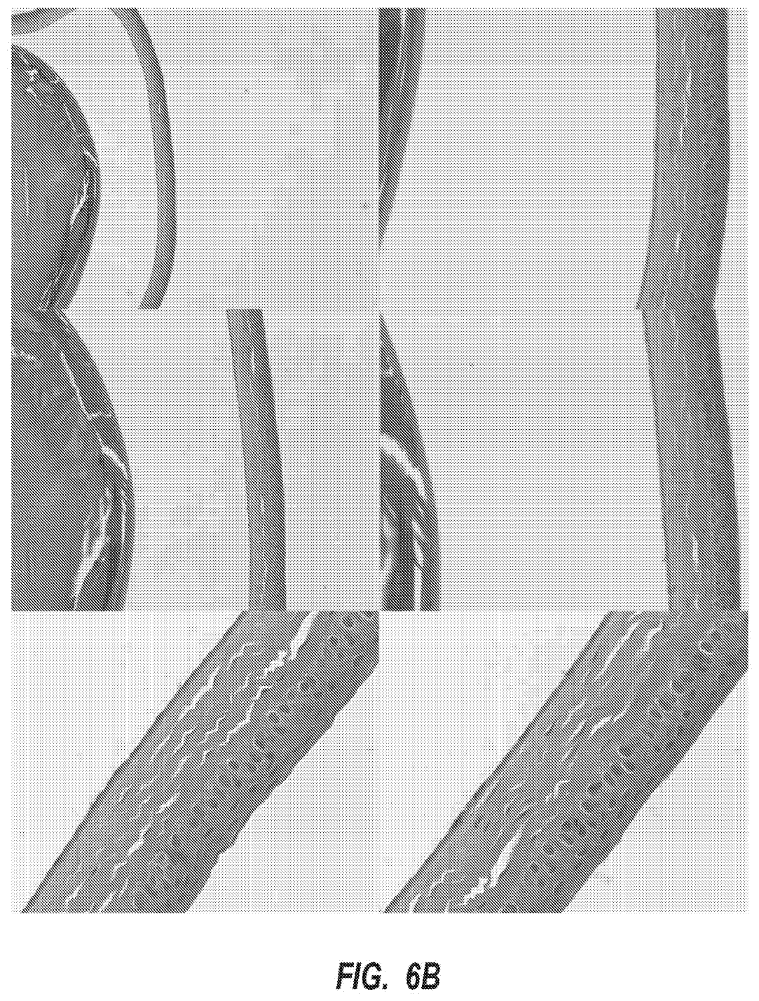

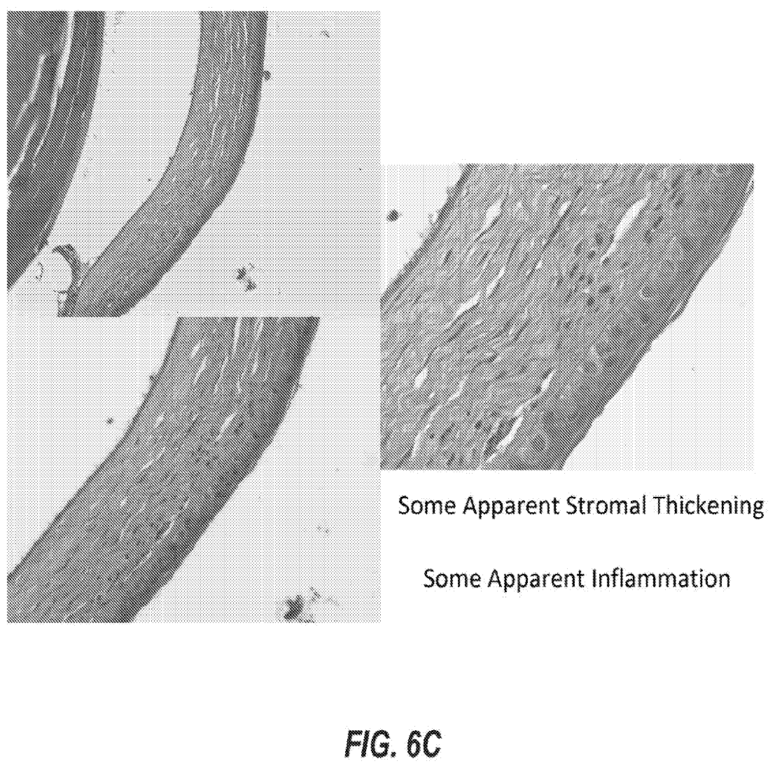

### DETAILED DESCRIPTION OF THE INVENTION

### Abbreviations and Definitions

Detailed descriptions of one or more embodiments are provided herein. However, the invention can be embodied in various forms. Therefore, specific details disclosed herein are not to be interpreted as limiting, but rather as a basis for the claims and as a representative basis for teaching one skilled in the art to employ the invention in any appropriate manner.

The singular forms “a”, “an” and “the” include plural reference unless the context dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is consistent with the common United States patent law definition of “comprising” and is understood to have an open term meaning “at least the following,” and also does not exclude additional features, limitations, aspects, etc. Thus, for example, “a process involving steps a, b, and c” means that the process includes at least steps a, b and c. Wherever the terms “a” or “an” are used, “one or more” is understood, unless it is nonsensical in context.

As used herein, “about” can be approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

An “effective amount”, “sufficient amount” or “therapeutically effective amount” can be an amount of a compound that is sufficient to effect beneficial or desired results, including clinical results. As such, the effective amount can be sufficient, for example, to reduce or ameliorate the severity and/or duration of an affliction or condition, or one or more symptoms thereof, prevent the advancement of conditions related to an affliction or condition, prevent the recurrence, development, or onset of one or more symptoms associated with an affliction or condition, or enhance or otherwise improve the prophylactic or therapeutic effect(s) of another therapy. An effective amount also includes the amount of the compound that avoids or substantially attenuates undesirable side effects.

“Treatment” can refer to an approach for obtaining beneficial or desired results, including clinical results. Beneficial or desired clinical results can include, but are not limited to, alleviation or amelioration of one or more symptoms or conditions, diminution of extent of disease, a stabilized (i.e., not worsening) state of disease, preventing spread of disease, delay or slowing of disease progression, amelioration or palliation of the disease state and remission (whether partial or total), whether detectable or undetectable. “Treatment” can also be prolonging survival as compared to expected survival if not receiving treatment.

The term “in need thereof” can refer to the need for symptomatic or asymptomatic relief from a condition such as, for example, an ocular condition, a skin condition, a lung condition, cancer or a neurodegenerative disease. The subject in need thereof may or may not be undergoing treatment for conditions related to, for example, an ocular condition, a skin condition, a lung condition, cancer, or a neurodegenerative disease. For example, in some embodiments, the cancer can be a retinoblastoma. Retinoblastoma is a malignant glioma and a rare form of cancer. It rapidly develops from the immature cells of the retina, the region of the eye tissue responsible for light-detection. It is the most common primary malignant intraocular cancer in children, almost exclusively found in young children that shows a hereditary pattern.

The term “carrier” can refer to a diluent, adjuvant, excipient, or vehicle with which a compound is administered. Non-limiting examples of such pharmaceutical carriers include liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used. Other examples of suitable pharmaceutical carriers are described in Remington: The Science and Practice of Pharmacy, 21^{st} Edition (University of the Sciences in Philadelphia, ed., Lippincott Williams & Wilkins 2005); and Handbook of Pharmaceutical Excipients, 7^{th} Edition (Raymond Rowe et al., ed., Pharmaceutical Press 2012); each hereby incorporated by reference in its entirety.

The terms “animal,” “subject,” and “patient” can refer to all members of the animal kingdom including, but not limited to, mammals, animals (e.g., cats, dogs, cows, horses, swine, etc.) and humans.

“Symptoms” can refer to one or more biological and/or physiological sequelae, including but not limited to hypersensitivity, burning, itching and light sensitivity, decrease in visual acuity, redness, pain, irritation, and photophobis. For example, symptoms of neovascularization comprise pain and redness.

“Agonist” can refer to a compound that can combine with a receptor, such as a serotonin receptor, to produce a cellular response. An agonist can be a ligand that directly binds to the receptor. Alternatively, an agonist can combine with a receptor indirectly by, for example, (a) forming a complex with another molecule that directly binds to the receptor, or (b) otherwise results in the modification of another compound so that the other compound directly binds to the receptor. An agonist can be referred to as an agonist of a particular serotonin receptor, such as a 5-HT2A serotonin receptor agonist.

Serotonin and the 5-HT2A Receptor

Serotonin (5 hydroxytryptamine; 5-HT) is a neurotransmitter and hormone whose effects are mediated through interactions at seven different families of receptor proteins, comprised of 14 different subtypes, consisting of 13 G-protein coupled receptors and one ligand-gated ion channel. Embodiments as described herein can comprise any of the receptor proteins of the seven different families of receptor proteins.

Serotonin is primarily known for its function as a neurotransmitter within the CNS, and is involved in many processes including cognition and memory. In the periphery, however, serotonin also plays significant roles where it mediates important processes like vasoconstriction and heart rate in the cardiovascular system, and gastrointestinal function. Although serotonin has been demonstrated to be involved in immune system function its precise role remains unclear.

Serotonin has been shown to influence a number of immunological processes, and can lead to both increases and decreases in proinflammatory cytokines. Examples of contradictory reports on 5-HT being proinflammatory and anti-inflammatory comprise Mossner R and Lesch K P (1998) Role of serotonin in the immune system and in neuroimmune interactions. Brain Behav Immun 12: 249-271; Kubera et al (2005) Effects of serotonin and serotonergic agonists and antagonists on the production of tumor necrosis factor alpha and interleukin-6. Psychiatry Res 134: 251-258; Kang et al. Regulation of serotonin-induced trafficking and migration of eosinophils. PLoS One 8; Diirk et al Production of serotonin by tryptophan hydroxylase 1 and release via platelets contribute to allergic airway inflammation. Am J Respir Crit Care Med 187: 476-485, 2013, each of which are incorporated herein by reference in their entireties.

There is a high level of expression of serotonin receptor 5-HT2A within the frontal cortex, with significant localization to the apical dendrites of cortical pyramidal cells (Willins et al., 1997), and further expression at lower levels throughout the brain (Nichols and Nichols, 2008). These receptors have been shown to participate in processes such as cognition and working memory, have been implicated in affective disorders such as schizophrenia, and have been shown to mediate the primary effects of hallucinogenic drugs (Nichols, 2004).

In addition, many peripheral tissues express 5-HT2A receptors. Within the vasculature, 5-HT2A receptors are known to modulate vasoconstriction (Nagatomo et al., 2004). Its role in other tissues such as mesangial cells of the kidney, fibroblasts, liver, and lymphocytes remains less defined, but has been linked to cellular proliferation and differentiation. Embodiments as described herein can comprise serotonin receptors, such as the 5-HT2A receptor, that are located in peripheral tissues. For example, peripheral tissues can comprise immunologically-restricted tissues.

In embodiments, the serotonin receptor comprises the 5-HT2A serotonin receptor. However, embodiments as described herein can comprise other receptor proteins of the family of serotonin receptors, such as 5-HT2B and 5-HT2C receptors, or downstream effector proteins activated by serotonin 5-HT2A receptors that convey the therapeutic effect to the cell or tissue.

Ocular Conditions

“Ocular tissue” can refer to a tissue contained within the eye. Ocular tissue includes without limitation tissues comprising cells of the lens, the cornea (endothelial, stromal and/or epithelial corneal cells), the iris, the retina, choroid, sclera, ciliary body, vitrous body, ocular vasculature, canal of Schlemm, ocular muscle cells, optic nerve, and other ocular sensory, motor and autonomic nerves.

The term “ocular condition” or “ocular disease” can refer to a disease or condition of one or more tissues, parts, or ocular regions of the eye that impairs the normal functioning of the eye. The anterior segment of the eye refers to the front third of the eyeball and includes structures located between the front surface of the cornea and the vitreous. The posterior segment of the eye refers to the rear two-thirds of the eyeball (behind the lens) and includes the vitreous, retina, optic disc, choroid, and pars plana. Non-limiting examples of an ocular condition comprise AMD, choroidal vascularization, diabetic retinopathies, viral retinopathies, glaucoma, corneal allograft transplant rejection, ocular hypertension, corneal neovascularization, keratoconjunctivitis, viral conjunctivitis, allergic ocnjunctivitisi, uveitis, iritis, keratitis, and infection.

The “eye” is the sense organ for sight, and includes the eyeball, or globe, the orbital sense organ that receives light and transmits visual information to the central nervous system. Broadly speaking, the eye includes the eyeball and the tissues and fluids which constitute the eyeball, the periocular muscles (such as the oblique and rectus muscles), and the portion of the optic nerve which is within or adjacent to the eyeball.

Physiological angiogenesis, neovascularization, and a normal immune system are required for embryonic development, tissue remodeling and wound healing. However, in certain tissues and diseases, dysregulation of these tightly controlled processes can result in pathological conditions, such as ocular conditions.

Pathological vascularization, dysregulation of vascular function, and hypersensitivity are critical determinates in the outcome of many ocular diseases and pathologies. For example, pathological vascularization is critical component to blinding stromal keratitis, proliferative retinopathies, and macular degeneration. Embodiments as described herein can treat diseases or symptoms of ocular vascularization-associated disease processes, such as in blinding stromal keratitis, proliferative retinopathies, and macular degeneration.

In diseases of the eye, pathological vascularization feeds a cascade of host-mediated responses that exacerbates the pathological processes within the innervated tissue and lowers the prognosis of disease resolution. Development of in vitro and in vivo vascularization-associated disease model systems have been expanded to additional pathological vascularization-associated diseases and provided opportunities to evaluate additional therapeutics, including serotonin receptor agonists such as the 5-HT agonist 2,5-Dimethoxy-4-iodoamphetamine (DOI). Findings indicate that in ocular models of disease, DOI potently inhibits disease-associated vascularization of tissues, thereby preventing the chronic pathology normally associated with disease progression.

Embodiments as described herein can be used to treat or ameliorate the symptoms associated with diseases of the eye. For example, dysregulation of vascularization processes or hypersensitivity can lead to vision-threatening ocular diseases or pathologies. In embodiments, a vascular-associated eye disease or hypersensitivity can be associated with, is caused by, or is exacerbated by vascular defects including but not limited to, angiogenesis, lymphangiogenesis, neovascularization, vascular leakage, edema, increased oxygen, ischemia, vasoconstriction, vasodialation, vascular occlusions, increased hypersensitivity reactions and/or ocular hypertension. Non-limiting examples of ocular diseases, such as vascularization-associated diseases of the eye, comprise Age-Related Macular Degeneration; choroidal vascularization; diabetic retinopathies; viral retinopathies; ocular hypertension; glaucoma; keratoconjuntivitis; conjunctivitis; herpetic stromal keratitis.

Embodiments as described herein can be used to reduce or ameliorate acute conditions or chronic ocular conditions. Further embodiments can prevent an acute condition from progressing to a chronic ocular condition. Embodiments as described herein can affect the TNF-axis, so as to reduce, ameliorate or prevent TNF-associated acute or chronic ocular conditions, or the progression thereof. Further embodiments as described herein can affect t IL17/IL22/IL23 axis, so as to reduce, ameliorate, or prevent acute conditions, chronic ocular conditions, or the progression of acute conditions to a chronic ocular condition. Furthermore, and without being bound by theory Further, embodiments as described herein can affect IL-6, IL-1, and/or IL-8 pathways.

Ocular Infection

“Ocular infection” can refer to an abnormal condition caused by bacteria, fungi, protozoa and/or viruses. Infections, if not treated, can lead to more severe ocular disorders.

Globally, infection- and inflammation-associated eye diseases are the leading causes of corneal blindness and visual morbidity, with over 500 million individuals affected. For example, Herpes Simplex virus type I (HSV-1), is present in 70-90% of the population and is the leading cause of corneal blindness in developed countries. The National Eye Institute estimates that 450,000 Americans have experienced some form of ocular herpetic disease, with 50,000 new and recurrent cases diagnosed. Current anti-pathogen drugs fail to inhibit pathogen-induced inflammatory responses. As such, approximately 25% of cases present with serious inflammation-associated stromal keratitis. Individuals that have experienced ocular herpes, have a 50% chance of recurrence. Each repeated episode triggers a chronic inflammatory disease process that that can result in vascularization and subsequent vision threatening scarring of the cornea that eventually requires corneal transplantation to resolve. Immuno-suppressive drugs, such as dexamethasone, can control deleterious hypersensitivity; however, they also license uncontrolled pathogen replication and are associated with loss of an intact corneal epithelial barrier, increased ocular pressure and eventual deterioration of vision. By contrast, modulation of 5-HT receptor activity within the eye has been shown to decrease ophthalmic pressure. Combined with its newly discovered anti-inflammatory and anti-vascularization properties, its potential within the eye can be immense, for example by replacing corticosteroids for several ocular disease indications. Embodiments as described herein can be used to reduce, or ameliorate, or prevent infection- and hypersensitivity hypersensitivity-associated eye diseases, non-limiting examples of which are described herein.

Embodiments as described herein are applicable to any ocular infection. In embodiments, the infection can be resolved. In other embodiments, the infection can never be resolved, such as is the case with a herpes viral infection. In this example, replication at the initial site of infection can be resolved, but the infection persists within a state of latency with sporadic episodes of reinfection. For example, it can be important to control the recurrent nature of a lifelong infection that reactivates from neurons to cause repeated bouts of ocular disease as seen in chronic Herpetic eye disease. Embodiments as described herein can control reactivation-mediated recurrent disease.

Embodiments as described herein can prevent reactivation of a latent virus, so as to prevent viral shedding, transmission, sporadic reinfection of tissues and subsequent recurrent acute disease and development of chronic disease manifestations.

In embodiments, the infection can be caused by a virus from the herpesvirus family, the adenovirus family, adenovirus, herpes virus, Cytomegalovirus, Parvovirus or varicella zoster virusParvovirusor a combination thereof.

Viral Reactivation

A viral infection is present in a host when a virus replicates itself within the host. A virus contains its own genetic material but uses the machinery of the host to reproduce. The virus can reproduce immediately, whereby the resulting virions destroy a host cell to attack additional cells. This process is the viral lytic cycle. Alternatively, a virus can establish a quiescent infection in a host cell, such as in a nerve or immune cell, lying dormant until environmental stimuli trigger re-entry into the lytic replication cycle. Such re-emergence or re-entry into the lytic replication cycle is termed reactivation.

“Viral latency” can refer to the ability of a virus, including pathogenic viruses such as HSV-1, to lie dormant (or latent) within a cell, such as a nerve cell. Latent viruses do not replicate and makes only few viral proteins. Such latent viral infection can be considered a persistant infection, which lasts for long periods of time and can occur when the primary infection is not cleared by the adaptive immune response. Examples of viruses that cause persistent infections comprise Varicella-zoster virus, measles virus, HIV-1, cytomegalovirus, Epstein Barr Virus, Kaposi's Sarcoma Herpesvirus, HSV-2, Adenovirus, HHV-6, HHV-7, Papillomavirus, Polyomavirus and HSV-1.

“Viral reactivation” or “virus reactivation” can refer to when a latent virus is reactivated into its active replicative phase, such as a result of an internal or external trigger. Non-limiting examples of such triggers comprise stress, hormonal, UV exposure, immunosuppression, immunocompromised, chemotherapies, drug induced, fevers, and age. “Viral shedding” can refer to the expulsion and release of virus progeny following successful lytic replication within a host-cell infection. The terms can refer to shedding from a single cell, shedding from one part of the body into another part of the body, and shedding from bodies into the environment where the viruses can infect other bodies.

“Viral transmission” can refer to the process by which viruses spread between hosts. For example, viral transmission includes spread to members of the same host species or spread to different species in the case of viruses that can cross species barriers.

Embodiments as described herein can prevent viral reactivation, shedding, transmission, or a combination thereof. For example, embodiments can prevent reactivation, shedding, and/or transmission of HSV from infected individuals, such as those infected with alphpaherpesvirus, such as HSV-1, VZV or HSV-2. In embodiments, such prevention of reactivation can prevent the development of secondary diseases and/or pathogenesis, such as ocular diseases, including those described herein.

Embodiments as described herein can maintain viral latency, so as to prevent viral reactivation.

Viral Retinopathy

“Retinopathy” can refer to a persistent or acute damage to the retina of the eye. In certain instances, the damage to the retina of the eye can cause loss of function of the eye. In certain instances, hypersensitivity and vascular remodeling can occur over prolonged periods of time unnoticed by the subject suffering from the pathology.

Retinopathies can be caused by diabetes mellitus, arterial hypertension, retinopathy of prematurity, radiation retinopathy, solar retinopathy, sickle cell disease, retinal vascular disease such as retinal veion or artery occlusion, trauma, or an infection, such as a viral infection. In embodiments, the retinopathies are viral retinopathies, and can be CMV- or VZV-associated.

Retinopathies are often proliferative, and can result from neovascularization.

Viral retinopathies comprise Cytomegalovirus (CMV)-associated retinopathis, such as CMV retinitis, and Varicella-Zoster Virus (VZV)-associated retinopathies.

Cytomegalovirus is a ubiquitous DNA virus that infects the majority of the adult population. In the immunocompetent host, infection can be asymptomatic or limited to a mononucleosis-like syndrome. Like many other herpesviruses, CMV remains latent in the host and can reactivate if host immunity is compromised.

In immunocompromised individuals, primary infection or reactivation of latent virus can lead to opportunistic infection of multiple organ systems. In the eye, CMV can present as a viral necrotizing retinitis. If left untreated, CMV retinitis inexorably progresses to visual loss and blindness.

Progressive outer retinal necrosis, also known as Varicella zoster virus retinitis (VZVR), is an aggressive, necrotizing inflammation of the eye's retina caused by herpes varicella zoster virus. It is found in people with advanced AIDS, but has also been reported in those who are severely immunocompromised due to chemotherapy. The majority of those with progressive outer retinal necrosis develop severe vision loss and blindness.

Diabetic Retinopathy

“Diabetic retinopathy” can refer to damage to the retina or disorders of the retina that is caused by diabetes. For example, the damage can be to the blood vessels in the retina of the eye which are vital to bringing oxygen and nutrients to the retina.

Diabetic retinopathy is the third leading cause of adult blindness (accounting for almost 7% of blindness in the USA), is associated with extensive angiogenic events. Nonproliferative retinopathy is accompanied by the selective loss of pericytes within the retina, and their loss results in dilation of associated capillaries dilation and a resulting increase in blood flow. In the dilated capillaries, endothelial cells proliferate and form outpouchings, which become microaneurysms, and the adjacent capillaries become blocked so that the area of retina surrounding these microaneurysms is not perfused. Eventually, shunt vessels appear between adjacent areas of micro aneurysms, and the clinical picture of early diabetic retinopathy with micro aneurysms and areas of nonperfused retina is seen. The microaneurysms leak and capillary vessels can bleed, causing exudates and hemorrhages. Once the initial stages of background diabetic retinopathy are established, the condition progresses over a period of years, developing into proliferative diabetic retinopathy and blindness in about 5% of cases. Proliferative diabetic retinopathy occurs when some areas of the retina continue losing their capillary vessels and become nonperfused, leading to the appearance of new vessels on the disk and elsewhere on the retina. These new blood vessels grow into the vitreous and bleed easily, leading to preretinal hemorrhages. In advanced proliferative diabetic retinopathy, a massive vitreous hemorrhage can fill a major portion of the vitreous cavity. In addition, the new vessels are accompanied by fibrous tissue proliferation that can lead to traction retinal detachment.

Diabetic retinopathy is associated primarily with the duration of diabetes mellitus; therefore, as the population ages and diabetic patients live longer, the prevalence of diabetic retinopathy will increase. Laser therapy is currently used in both nonproliferative and proliferative diabetic retinopathy. Focal laser treatment of the leaking microaneurysms surrounding the macular area reduces visual loss in 50% of patients with clinically significant macular edema. In proliferative diabetic retinopathy, panretinal photocoagulation results in several thousand tiny burns scattered throughout the retina (sparing the macular area); this treatment reduces the rate of blindness by 60 percent. Early treatment of macular edema and proliferative diabetic retinopathy prevents blindness for 5 years in 95% of patients, whereas late treatment prevents blindness in only 50 percent. Therefore, early diagnosis and treatment are essential.

Age-Related Macular Degeneration

“Macular Degeneration” can refer to the degeneration of the macula, which is a small yellow area on the back of the eye and located in the middle of the retina. Because of the position of the macula (the center of the retina), the resulting vision loss in Macular Degeneration is the central vision. In many cases, people suffering from Age-Related Macular Degeneration have normal peripheral vision, but generate a blind spot right in the middle of their sight path. Therefore, Macular Degeneration can affect one's ability to read, drive and recognize faces.

Age-related macular degeneration (AMD), a disease that affects approximately one in ten Americans over the age of 65. AMD is characterized by a series of pathologic changes in the macula, the central region of the retina, which is accompanied by decreased visual acuity, for example affecting central vision. AMD involves the single layer of cells called the retinal pigment epithelium that lies immediately beneath the sensory retina. These cells nourish and support the portion of the retina in contact with them, i.e., the photoreceptor cells that contain the visual pigments. The retinal pigment epithelium lies on the Bruch membrane, a basement membrane complex which, in AMD, thickens and becomes sclerotic. New blood vessels can break through the Bruch membrane from the underlying choroid, which contains a rich vascular bed. These vessels can in turn leak fluid or bleed beneath the retinal pigment epithelium and also between the retinal pigment epithelium and the sensory retina. Subsequent fibrous scarring disrupts the nourishment of the photoreceptor cells and leads to their death, resulting in a loss of central visual acuity. This type of age-related maculopathy is called the “wet” type because of the leaking vessels and the subretinal edema or blood. The wet type accounts for only 10% of age-related maculopathy cases but results in 90% of cases of legal blindness from macular degeneration in the elderly. The “dry” type of age-related maculopathy involves disintegration of the retinal pigment epithelium along with loss of the overlying photoreceptor cells. The dry type reduces vision but usually only to levels of 20/50 to 20/100.

AMD is accompanied by distortion of central vision with objects appearing larger or smaller or straight lines appearing distorted, bent, or without a central segment. In the wet type of AMD, a small detachment of the sensory retina can be noted in the macular area, but the definitive diagnosis of a subretinal neovascular membrane requires fluorescein angiography. In the dry type, drusen can disturb the pigmentation pattern in the macular area. Drusen are excrescences of the basement membrane of the retinal pigment epithelium that protrude into the cells, causing them to bulge anteriorly; their role as a risk factor in age-related maculopathy is unclear. No treatment currently exists for the dry type of age-related maculopathy. Laser treatment is used in the wet type of age-related maculopathy and initially obliterates the neovascular membrane and prevents further visual loss in about 50% of patients at 18 months. By 60 months, however, only 20% still have a substantial benefit.

Neovascular Glaucoma

Neovascular glaucoma is a pathological condition wherein new capillaries develop in the retina or iris of the eye. In the iris, for example, the angiogenesis usually originates from vessels located at the pupillary margin, and progresses across the root of the iris and into the trabecular meshwork. Fibroblasts and other connective tissue elements are associated with the capillary growth and a fibrovascular membrane develops which spreads across the anterior surface of the iris. Eventually this tissue reaches the anterior chamber angle where it forms synechiae. These synechiae in turn coalesce, scar, and contract to ultimately close off the anterior chamber angle. The scar formation prevents adequate drainage of aqueous humor through the angle and into the trabecular meshwork, resulting in an increase in intraocular pressure that can result in blindness.

Neovascular glaucoma can occur as a complication of diseases in which retinal ischemia is predominant. About one third of the patients with this disorder have diabetic retinopathy and 28% have central retinal vein occlusion. Other causes include chronic retinal detachment, end-stage glaucoma, carotid artery obstructive disease, retrolental fibroplasia, sickle-cell anemia, intraocular tumors, and carotid cavernous fistulas. In its early stages, neovascular glaucoma can be diagnosed by high magnification slitlamp biomicroscopy, where it reveals small, dilated, disorganized capillaries (which leak fluorescein) on the surface of the iris. Later gonioscopy demonstrates progressive obliteration of the anterior chamber angle by fibrovascular bands. While the anterior chamber angle is still open, conservative therapies can be of assistance. However, once the angle closes surgical intervention is required in order to alleviate the pressure.

Conjunctivitis

Conjunctivitis can refer to a hypersensitivity of the conjunctiva. The conjunctiva is the thin clear tissue that lies over the white part of the eye and lines the inside of the eyelid. Conjunctivitis has a number of different causes, including viruses and bacteria (such as gonorrhea or chlamydia). Conjunctivitis caused by some bacteria and viruses can spread easily from person to person.

Conjunctivitis caused by bacteria (bacterial conjunctivitis), including those related to STDs, can be treated with embodiments as described herein. For example, embodiments can be in the form of eye drops, ointments, or pills. Eye drops or ointments can be applied to the inside of the eyelid daily, such as three to four times a day for five to seven days. As another example, pills can be taken for several days.

Conjunctivitis caused by viruses (viral conjunctivitis) often results from the viruses that cause a common cold, such as Rhinovirus or Adenovirus. Adenovirus, for example, can cause the ocular defects that are most associated with conjunctivitis, or pink eye. Viral conjunctivitis can be highly contagious. Some viruses cause scarring of the cornea.

Allergic conjunctivitis is an eye hypersensitivity disease caused by an allergic reaction to foreign substances, such as pollen or mold spores. Allergic conjunctivisitis can be acute allergic conjunctivitis, which is a short-term condition common during allergy season, or chronic allergic conjunctivitis, which is a less common condition that can occur year round. Chronic allergic conjunctivitis is characterized by symptoms which come and go, comprising burning, itching and light sensitivity.

Embodiments as described herein can treat, reduce, or ameliorate the pathogenesis and symptoms of allergic conjunctivitis. In other embodiments, the recurrence of chronic allergic conjunctivitis can be prevented.

Trauma

“Trauma” can refer to an injury to a structure or tissue of a subject, such as an eye, by a foreign object, blunt force trauma, or chemical. “Ocular trauma” for example refers to an injury to a structure or tissue of a subject's eye. Non-limiting examples of ocular traumas comprises surgical trauma, chemical trauma, blunt force such as trauma that results from rejection of transplanted tissue, chemical trauma, eye wall injury, such as closed globe injury or open globe injury, contusion, lamellar laceration, rupture, penetrating injury, intraocular foreign body injury, or perforating injury. can be caused by surgery, injury, accident. Ocular traumas can induce pathogenesis processes such as those seen in an ocular infection, such as hypersensitivity and neovascularization. An example of a surgical trauma comprises host-graft diseases that is caused by vascularization and hypersensitivity of newly transplanted tissues. Embodiments as described herein can treat, reduce, or ameliorate the pathogenesis associated with ocular traumas.

Allograft Transplant Rejection

“Transplantation” can refer to the process of taking a cell, tissue, or organ, called a “transplant” or “graft” from one individual (such as a donor individual) and placing it or them into a different individual. The individual who provides the transplant is called the “donor” and the individual who received the transplant is called the “host” (or “recipient”). In other embodiments, the transplant can be taken from and placed back into the same individual. An organ, or graft, transplanted between two genetically different individuals of the same species is called an “allograft”. A graft transplanted between individuals of different species is called a “xenograft”.

“Transplant rejection” can refer to a functional and structural deterioration of the organ due to an active immune response expressed by the recipient, and independent of non-immunologic causes of organ dysfunction.

The term “transplant rejection” can encompass both acute and chronic transplant rejection.

Transplant rejection occurs when transplanted tissue is rejected by the recipient's immune system, which destroys the transplanted tissue.

For example, corneal transplantation can result in corneal graft rejection, which is a specific immunological response of the host to the donor corneal tissue. Symptoms of corneal transplant rejection comprise decrease in visual acuity, redness, pain, irritation, and photophobis. Clinical signs of graft rejection comprise corneal edema, keratic precipitates on the corneal graft, corneal vascularization, stromal infiltrates, among others.

Transplant rejection can be HSV-induced or induced because of a Type IV hypersensitivity.

HSV has a natural ability to establish life long latency, and reactivation of latent infection can lead to recurrent disease. Recurrences of herpes simplex virus (HSV) can lead to corneal stromal scarring and decreased visual acuity. Consequently, herpetic stromal keratitis is a common indication for corneal transplantation. However, there is a relatively high risk of graft failure in this patient group.

Embodiments as described herein can treat, prevent, reduce, or ameliorate symptoms or pathogenesis associated with allograft transplant rejection, such as corneal transplant rejection.

Non-Ocular Conditions

“Non-ocular tissue” or “non-ocular disease” refers to any tissue or disease that is not of the eye. For example, a non-ocular infection comprises any infection of a tissue that is not an ocular tissue, such as an infection of the lung. Non-limiting examples of non-ocular conditions comprise conditions of the skin (such as plaque psoriasis) or conditions of the lung (such as pulmonary influenza) and conditions that can affect multiple non-ocular tissues, such as host-graft disease or a non-ocular infection.

Non-Ocular Infections

The terms “microbe” and “pathogen” can be used interchangeably herein and refer to any one of a variety of infectious microorganisms including, but are not limited to, bacterial, viral, protozoal, or fungal infectious agents. Two microbes are considered distinct if they belong to different classes or types of microorganisms, different subtypes or species within the same type, or different strains within the same subtype or species. Common infectious bacteria include, but are not limited to, Escherichia coli, Salmonella, Shigella, Klebsiella, Pseudomonas, Listeria monocytogenes, Mycobacterium tuberculosis, Mycobacterium avium-intracellulare, Yersinia, Francisella, Pasteurella, Brucella, Clostridia, Bordetella pertussis, Bacteroides, Staphylococcus aureus, Streptococcus pneumonia, B-Hemolytic strep., Corynebacteria, Legionella, Mycoplasm, Ureaplasma, Chlamydia, Neisseria gonorrhea, Neisseria meningitides, Hemophilus influenza, Enterococcus faecalis, Proteus vulgaris, Proteus mirabilis, Helicobacter pylori, Treponema palladium, Borrelia burgdorferi, Borrelia recurrentis, Rickettsial pathogens, Nocardia, and Acitnomycetes. Infectious respiratory bacteria include, but are not limited to, Streptococcus pneumoniae, Haemophilus influenzae, Staphylococcus aureus, klebsiella, or legionella. Common infectious viruses include, but are not limited to, influenza viruses, human immunodeficiency virus, human T-cell lymphocytotrophic virus, hepatitis viruses, Epstein-Barr Virus, cytomegalovirus, human papillomaviruses, orthomyxo viruses, paramyxo viruses, adenoviruses, corona viruses, rhabdo viruses, polio viruses, toga viruses, bunya viruses, arena viruses, rubella viruses, and reo viruses. Infectious respiratory viruses include, but are not limited to, influenza virus type A, influenza virus type B, influenza virus type C, parainfluenza virus type 1, parainfluenza virus type 2, parainfluenza virus type 3, rhinoviruses, respiratory syncytial virus, a respiratory coronavirus, or a respiratory adenovirus. Common infectious fungi include, but are not limited to, Cryptococcus neaformans, Blastomyces dermatitidis, Histoplasma capsulatum, Coccidioides immitis, Paracoccicioides brasiliensis, Candida albicans, Aspergillus fumigautus, Phycomycetes (Rhizopus), Sporothrix schenckii, Chromomycosis, and Maduromycosis. Infectious respiratory fungi include, but are not limited to, Coccidiodes immitus, Histoplasma capsulatum or Cryptococcus neoformans.

Embodiments as described herein can be used to treat disease and symptoms thereof of non-ocular infections. Further, embodiments can be used to reduce or ameliorate pathogenesis associated with non-ocular infections. Such diseases, symptoms, and pathogenesis are described herein.

Influenza

As used herein, the term “influenza” refers to an acute contagious respiratory disease resulting from infection by an influenza virus, including but is not limited to, a human or avian influenza virus. The term “influenza” encompasses all known types and subtypes of influenza viruses. Human influenza viruses are classified into three types (A, B and C) based on their immunologically distinct nucleoprotein (NP) and matrix (Ml) protein antigens. Influenza A (subtypes H1N1, H3N2, H5N1 and H7N7) is associated with high morbidity and mortality, has the potential to cause pandemics, and is virulet in patients of all ages. Substantial genetic differences exists amongst the various subtypes of human influenza A virus, all of which are known to infect both humans and birds. Avian influenza viruses, which infect birds, encompass various subtypes, each of which comprises multiple strains of varying pathogenicity. Avian influenza H5 and H9 viruses, for example, are classified as “low pathogenic” viruses, whereas H7 is a “high pathogenic” viruses.

Psoriasis

“Psoriasis” refers to a non-contagious skin condition characterized by inflamed lesions covered with scabs of dead skin. Psoriasis can occur on the elbows, knees, trunk, and scalp.

Psoriasis is an autoimmune disorder, and can be characterized by neovascularization, such as inflammation-induced vascularization.

Plaque psoriasis, e most common form of the disease, is characterized by small, red bumps that enlarge, become inflamed, and form scales. The top scales flake off easily and often, but those beneath the surface of the skin clump together. Removing these scales exposes tender skin, which bleeds and causes the plaques (inflamed patches) to grow.

Plaque psoriasis can develop on any part of the body, but most often occurs on the elbows, knees, scalp, and trunk. Other types of psoriasis comprise scalp psoriasis, nail psoriasis, guttate psoriasis, pustular psoriasis, palomar-plantar pustuulosis, acrodermatitis continua of Hallopeau, inverse psoriasis, erythrodermic psoriasis, and psoriatic arthritis.

The importance attributed to angiogenesis in psoriasis has grown significantly. The vascular network found within these lesions is highly altered, especially in the papillary dermis which is infiltrated by a large number of tortuous and dilated capillaries. Endothelial cells composing these vessels are activated and express many adhesion molecules promoting leukocyte recruitment (ICAM-1, VCAM-1, Thy-1, E- and P-selectin). Thus, this pathological angiogenesis is not a mere consequence of the disease, but a key component promoting leukocyte accumulation, inflammation and therefore, skin lesions.

Embodiments as described herein can treat disease or reduce the symptoms associated with psoriasis. Further, embodiments as described herein can reduce or ameliorate the pathogenesis associated with psoriasis, such as hypersensitivities, neovascularization, or a combination thereof.

Graft-Versus-Host Disease

Graft-versus-host disease (GvHD) is a medical complication following the receipt of transplanted tissue, for example from a genetically different person.

As used herein, “transplant rejection” or variations thereof refers to the host's immune system mounting an immune response to the graft, ultimately resulting in the graft being rejected by the host. Two types of “transplant rejection” comprise graft-versus-host disease and host-versus-graft disease.

As used herein, the term “graft-versus-host disease” refers to is an immune attack on the recipient by cells from a donor, often leading to rejection of the transplanted cells. Whilst the transplanted cells can be of any cell type, transplanted tissues that house enough immune cells to cause graft versus host disease include the blood and the bone marrow.

As used herein, the term “host-versus-graft disease” refers to the lymphocyte-mediated reactions of a host against allogeneic or xenogeneic cells acquired as a graft or otherwise, which lead to damage or/and destruction of the grafted cells. This is the common basis of graft rejection.

Angiogenesis can precede infiltration of transplanted tissue of inflammatory leukocytes during GVHD (see Initiation of acute graft-versus-host disease by angiogenesis

Katarina Riesner, Yu Shi, Angela Jacobi, Martin Kraeter, Martina Kalupa, Aleixandria McGearey, Sarah Mertlitz, Steffen Cordes, Jens-Florian Schrezenmeier, Jorg Mengwasser, Sabine Westphal, Daniel Perez-Hernandez, Clemens Schmitt, Gunnar Dittmar, Jochen Guck, Olaf Penack Blood January 2017, blood-2016-08-736314; DOI: 10.1182/blood-2016-08-736314; incorporated herein in its entirety).

Embodiments as described herein can treat disease or reduce the symptoms associated with graft-versus-host disease. Further, embodiments as described herein can reduce or ameliorate the pathogenesis associated with graft-versus-host disease, such as hypersensitivities, neovascularization, or a combination thereof.

Trauma

As described herein, “trauma” can refer to an injury to a structure or tissue of a subject by a foreign object, blunt force trauma, or chemical. “Dermal trauma” for example refers to an injury to a structure or tissue of a subject's skin. Non-limiting examples of traumas comprises surgical trauma, chemical trauma, blunt force such as trauma that results from rejection of transplanted tissue, chemical trauma, contusion, rupture, penetrating injury, foreign body injury, or perforating injury, and can be caused by surgery, injury, accident. Traumas can induce pathogenesis processes such as those seen in infection, such as hypersensitivity and neovascularization. An example of a surgical trauma comprises host-graft diseases that is caused by vascularization and hypersensitivity of newly transplanted tissues. Embodiments as described herein can treat, reduce, or ameliorate the pathogenesis associated with traumas, including non-ocular traumas.

Pathogenesis

“Pathogenesis” can refer to the mode of origin, biological mechanism(s), or development of disease or condition. For example, pathogenesis can refer to hypersensitivity, angiogenesis, for example of blood vessels or lymphatic vessels; vascularization; vascular occulsions; vascular leakage; vascular permeability; angiogenesis; lymphangiogenesis; neovascularization; vasodialation; vasoconstriction, for example that of lymphatics or blood vessels; vascular occlusions; edema; corneal epithelial defects; increased intraocular pressure; increased oxygen saturation; ischemia; haemorrhage; necrotizing inflammation; epithelial hyperproliferation; epithelial thickening; fibrosis; or a combination thereof.

Embodiments as described herein can reduce, ameliorate, or prevent pathogenesis associated with an ocular or non-ocular disease. In some embodiments, the pathogenesis is chronic pathogenesis, and persists after the acute disease itself is resolved. Non-limiting examples of ocular pathogenesis comprise hypersensitivity hypersensitivity, angiogenesis, neovascularization, vascular leakage, vascular permeability, or a combination thereof.

Pathological vascularization and dysregulation of vascular function are main contributors to all infectious and many non-infectious disease processes in ocular tissue Embodiments as described herein can be used to reduce, ameliorate, or inhibit vascularization, such as neovascularization, in an ocular tissue of a subject.

Embodiments as described herein can reduce, ameliorate or prevent symptoms associated with vascularization in an ocular or non-ocular tissue of a subject. Non-limiting examples of such symptoms comprise conjunctivitis, keratoconjunctivitis, hypertension, glaucoma, macular degeneration, or edema.

In embodiments, the vascularized tissue can comprise an ocular tissue, such as a tissue of the eye, or can comprise a non-ocular tissue, such as a tissue of the lung.

In embodiments, neovascularization can refer to any type of angiogenesis or new vascularization of tissues. For example, vascularization can refer to angiogenesis of a blood vessel, angiogenesis of a lymphatic vessel, or a combination thereof.

Lymphangiogenesis plays key roles in regulating hypersensitivity, tissue edema, intraocular pressure and hypersensitivity disease processes.

Non-limiting markers of vascularization and/or lymphangiogenesis comprise LYVE, VEGFA, VEGFB, VEGFC, VEGFD, VEGFR-3, PROX1, CCL21, TNF, IL-6, Angiopioetin 1, Angiopioetin 2, FLT-1, KDR, Tie-1, HIF1a, PGF, FGF, IL8, IL1B, IFN, TGF, IL17, TIMP, MMP2, MMP9, and NOTCH. In embodiments, neovascularization can be scored on a grading scale. For example, a three point scale can be used in a rabbit model, and a 16 point scale can be used in mice. Such scales allow for more accuracy in the assessment of neovascularization. For example, corneal neovascularization can be evaluated as previously described in Rajasagi et al. 2011; J Immunol 186:1735, which is incorporated herein in its entirety, using a scale of 0 to 16, where each of the four quadrants of the eye was evaluated for the density of vessels that have grown onto the cornea and the extent of neovessels. According to this system, the score of the four quadrants of the eye (between 0, indicating the absence of vessels, to 4, meaning maximal density of new vasculature) were then summed to derive the neovascularization index (a total range of 0-16) for each eye at a given time point.

Embodiments as described herein can be used to reduce, prevent, or ameliorate hypersensitivity. For example, the hypersensitivity can be ocular hypersensitivity, or it can be a hypersensitivity that affects an immunologically-restricted tissue, such as that of the lung. Hypersensitivity refers to a localized protective reaction of tissue to irritation, injury, infection, or disease, and is characterized by pain, redness, swelling, and potentially loss of function. Non-limiting markers of inflammatory disease comprise TNF, IL-6, IL8, IL1B, IllA, IL12, IFNa, IFNb, IFNg, TGF, IL17, IL20, IL22, LTA, IL23, IL18, CCL2, CCL5, CCL3, CCL4, CCL11, CD11a, CD3, CD4, CD8, and CRP.

Embodiments as described herein can be used to reduce, prevent, or ameliorate vascular leakage. Vascular leakage refers to the permeability of vessels and capillaries that can result in increased influx of immune cells causing hypersensitivity of tissue, formation of edema, or leakage of blood cells into tissue. Vascular leakage can also be referred to as vascular permeability. Vascular leakage can be the one way flow of cells or fluid, or can be the two way flow of cells or fluid.

Embodiments as described herein can be used to reduce, prevent, or ameliorate ocular pressure, for example hypertension in the eye. Intraocular pressure refers to the pressure of the fluid inside the eye as measured by a tonometer that is a result of several causes including excessive aqueous humor production, inadequate drainage of fluids within the eye through lymphatics, trauma, medications, hypersensitivity and/or infection. Ocular hypertension refers to an intraocular pressure greater than 21 mm Hg.

Embodiments as described herein can reduce, ameliorate, or inhibit corneal epithelial thickening or loss. For example, loss of epithelia can occur during acute traumatic events. In certain instance, epithelial thickening can occur following an acute traumatic event as a chronic response.

In embodiments, clinical diseases, for example stromal disease, corneal opacity, and ocular hypersensitivity, are scored according to a grading scale. For example, the scale can be a three point scale (from 0 to 3) and comprise the parameters that are documents in Hill et al, The antimicrobial agent C31G is effective for therapy for HSV-1 ocular keratitis in the rabbit eye model. Antiviral Res. 2013 October; 100(1):14-9 and Clement et al. Clinical and antiviral efficacy of an ophthalmic formulation of dexamethasone povidone-iodine in a rabbit model of adenoviral keratoconjunctivitis. Invest Ophthalmol Vis Sci. 2011 Jan. 21; 52(1):339-44, both of which are incorporated herein in their entireties.

In embodiments, clinical scoring of slit lamp biomicroscopy can be visualized using a fluorphore enhance slit lamp biomicroscope. In embodiments, this can be scored on a grading scale, such as a 4 point scale (from 0 to 4), as detailed within Hill et al, The antimicrobial agent C31G is effective for therapy for HSV-1 ocular keratitis in the rabbit eye model. Antiviral Res. 2013 October; 100(1):14-9 and Clement et al. Clinical and antiviral efficacy of an ophthalmic formulation of dexamethasone povidone-iodine in a rabbit model of adenoviral keratoconjunctivitis. Invest Ophthalmol Vis Sci. 2011 Jan. 21; 52(1):339-44, both of which are incorporated herein in their entireties.

Hypersensitivity refers to a set of undesirable reactions produced by a subject's normal immune system. For example, hypersensitivity can refer to an over-reaction of the immune system of a subject, and such over reaction can be damaging or uncomfortable. In embodiments, hypersensitivity requires a pre-sensitized state of the host. Hypersensitivity can be classified as Type I (Immediate), Type II (Antibody mediated), Type III (Immune Complex-mediated), and Type IV (Cell-mediated).

Type I hypersensitivity can be characterized by IgE binding to mast cells or basophils, causing degranulation of mast cells or basophil and release of reactive substances, such as histamines. Ocular Type I hypersensitivities, for example, can apply to immediate hypersensitivities that result in an increase in vascular permeability and migration of eosinophils and neutrophils, non-limiting examples of which comprise allergic responses, acute hemorrhagic conjunctivitis, atopic keratoconjunctivitis, bacterial conjunctivitis, emergent treatment of acute conjunctivitis, epidemic keratoconjunctivitis, giant papillary conjunctivitis, keratoconjunctivitis sicca, neonatal conjunctivitis, superior limbic keratoconjunctivitis, and viral conjunctivitis.

Type II hypersensitivity can be characterized by an antigen causing formation of IgM and IgG antibodies that bind to target cells when, when combined with action of a complement, can destroy the target cell.

Type III hypersensitivity can be characterized by antibodies and antigens that form complexes that cause damaging inflammation.

Type IV hypersensitivity can be characterized by a delayed T cell-mediated response that elicits production of cytokines and chemokines, as well as direct killing of target cells. Non-limiting examples of which comprise herpetic keratitis, keratoconjunctivitis, corneal transplant allograft rejection, contact dermatitis of the eye, and drug allergies. In one example, such as in herpetic stromal keratitis, a CD4 helper T cell response contributes to disease through T cell mediated responses. In embodiments, both Tc and Th play a role in the Type IV hypersensitivity.

Embodiments as described herein can comprise Type I, Type II, Type III, Type IV hypersensitivities or a combination thereof.

Other embodiments can comprise Type I hypersensitivities. Ocular Type I hypersensitivities, for example, can apply to immediate hypersensitivities that result in an increase in vascular permeability and migration of eosinophils and neutrophils, non-limiting examples of which comprise allergic responses, acute hemorrhagic conjunctivitis, atopic keratoconjunctivitis, bacterial conjunctivitis, emergent treatment of acute conjunctivitis, epidemic keratoconjunctivitis, giant papillary conjunctivitis, keratoconjunctivitis sicca, neonatal conjunctivitis, superior limbic keratoconjunctivitis, and viral conjunctivitis.

Still other embodiments can comprise Type IV hypersensitivities. Ocular Type IV hypersensitivities, for example, can apply to delayed T cell-mediated hypersensitivities, non-limiting examples of which comprise keratoconjunctivitis, corneal transplant allograft rejection, contact dermatitis of the eye, drug allergies, and herpes chronic responses.

Type IV hypersensitivity can be characterized by antigens activating Tc that kill target cells.

Any hypersensitivity in a tissue of the eye is associated with a disease of the the eye, as the eye largely an immune privileged tissue. As such, any hypersensitivity can change vision, have deleterious outcomes, or a combination thereof. Non-limiting examples of hypersensitivity processes that contribute to disease of the eye comprise inflammation, neovasularization, immune cell infiltration, infiltration of macrophages, infiltration of polymorphonuclear leukocytes (PMNs), infiltration of CD8 T cells, infiltration of CD4 T cells, inflammatory cytokine production, chemokine production, vascular leakage, edema, ulceration, increased intraocular pressure, tissue damage, and fibrosis.

Agonists

“Agonist” can refer to a compound that can combine and/or interact with a receptor, such as a serotonin receptor, to produce a cellular response. An agonist can be a ligand that directly binds to the receptor. Alternatively, an agonist can combine with a receptor indirectly by, for example, (a) forming a complex with another molecule that directly binds to the receptor, or (b) otherwise resulting in the modification of another compound so that said compound directly binds to the receptor. An agonist can be referred to as an agonist of a particular serotonin receptor, such as a 5-HT2A serotonin receptor agonist.

The term “5-HT_{2A} agonists” can refer to any compound or ligand that increases the activity of a 5-hydroxytryptamine 2A receptor. Non-limiting examples of such agonists include, but are not limited to, DOI (±)-1-(2,5-dimethoxyphenyl)-2-aminopropane hydrochloride; (R)-DOI ((R)-1-(2,5-dimethoxy-4-iodophenyl)-2-aminopropane) (greater than 95% R enantiomer); LA-SS-Az (2'S,4'S)-(+)-9,10-Didehydro-6-methylergoline-83-(trans-2,4-dimethylazetidide); 2C-BCB (TCB2) (4-Bromo-3,6-dimethoxybenzocyclobuten-1-yl) methylamine; and lysergic acid diethylamide (LSD).

Non-limiting examples of serotonin receptor agonists can be found in Nichols, et al, WIREs Membr Transp Signal 2012, which is incorporated herein in its entirety.

In embodiments, the serotonin receptor agonist can be a Phenethylamine, a Tryptamine, an Ergoline, or a combination thereof. Non-limiting examples of a Phenethylamine comprises 1-(4-Iodo-2,5-dimethoxyphenyl)propan-2-amine (DOI), 1-(4-bromo-2,5-dimethoxyphenyl)propan-2-amine (DOB), 1-(4-methyl-2,5-dimethoxyphenyl)propan-2-amine (DOM), 1-(2,5-Dimethoxy-4-nitrophenyl)propan-2-amine (DON), 2-(4-Iodo-2,5-dimethoxyphenyl)ethan-1-amine (2CI), 4-Bromo-2,5-dimethoxyphenylethanamine (2CB), 1-(3,4,5-Trimethoxyphenyl)propan-2-amine (TMA), 2-(3,4,5-trimethoxyphenyl)ethanamine (Mescaline), 1-[2,5-Dimethoxy-4-(trifluoromethyl)phenyl]propan-2-amine (DOTFM), (2R)-1-[4-(trifluoromethyl)-2,3,6,7-tetrahydrofuro[2,3-f][1]benzofuran-8-yl]propan-2-amine (TFMFly), and 25CINMoMe.

Non-limiting examples of a Tryptamine comprises DMT, [3-(2-Dimethylaminoethyl)-1H-indol-4-yl] dihydrogen phosphate (Psilocybin), 3-[2-(Dimethylamino)ethyl]-1H-indol-4-ol (Psilocin), and 5MEO-DMT.

In embodiments, the serotonin receptor agonist is an indazole compound, such as (S)-2-(8,9-dihydro-7H-pyrano[2,3-g]indazol-1-yl)-1-methylethylamine (AL-38022A).

Non-limiting examples of an Ergoline comprises 6aR,9R)—N,N-diethyl-7-methyl-4,6,6a,7,8,9-hexahydroindolo-[4,3-fg]quinoline-9-carboxamide (LSD), 1,1-Diethyl-3-(7-methyl-4,6,6a,7,8,9-hexahydro-indolo[4,3-fg]quinolin-9-yl)-urea (Lisuride), and (6aR,9R)-5-bromo-N,N-diethyl-7-methyl-4,6,6a,7,8,9-hexahydroindolo[4,3-fg]quinoline-9-carboxamide (Bromo-LSD; BOL).

In embodiments, the composition comprises a compound having the following chemical formula (II):

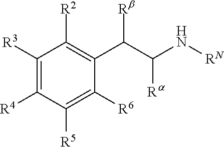

where non-limiting exemplary values of the R groups in the above substituted chemical structure are represented in the following table (Table 1):

| Name | R² | R³ | R⁴ | R⁵ | R⁶ | R^{α} | R^{β} | R^{N} |
|---|---|---|---|---|---|---|---|---|
| Mescaline | H | OCH₃ | OCH₃ | OCH₃ | H | H | H | H |
| TMA | H | OCH₃ | OCH₃ | OCH₃ | H | CH₃ | H | H |
| TMA-2 | OCH₃ | H | OCH₃ | OCH₃ | H | CH₃ | H | H |
| β- | OCH₃ | H | Br | OCH₃ | H | CH₃ | OCH₃ | H |
| methoxyDOB | | | | | | | | |
| DOM | OCH₃ | H | CH₃ | OCH₃ | H | H | H | H |
| DOB | OCH₃ | H | Br | OCH₃ | H | H | H | H |
| DOI | OCH₃ | H | I | OCH₃ | H | H | H | H |
| Sulfur analog | H | OCH₃ | OCH₃ | SCH₃ | H | H | H | H |
| of mescaline | | | | | | | | |
| Sulfur analog | H | OCH₃ | SCH₃ | OCH₃ | H | H | H | H |
| of mescaline | | | | | | | | |
| DOIB | OCH₃ | H | CH₂CH(CH₃)₂ | OCH₃ | H | CH₃ | H | H |
| DOTFM | OCH₃ | H | CF₃ | OCH₃ | H | CH₃ | H | H |

In some embodiments, R² of formula (II) can be OH, O—(C₁-C₆-alkyl), —O—(C₂-C₆-alkyl)-N(R⁵)₂, or —O—(C₂-C₆-alkyl)-N(R^{x})₃⁺halogen⁻; R³ of formula (II) can be OH, O—(C₁-C₆-alkyl), —O—(C₂-C₆-alkyl)-N(R^{x})₂, or —O—(C₂-C₆-alkyl)-N(R^{x})₃⁺halogen⁻; R⁴ of formula (II) can be halogen, C₁-C₂-haloalkyl, H, C₁-C₆-alkyl, C₁-C₆-alkyl sulfide, OH, O—(C₁-C₆-alkyl),—O—(C₂-C₆-alkyl)-N(R^{x})₂, or —O—(C₂-C₆-alkyl)-N(R^{x})₃⁺halogen⁻; R⁵ of formula (II) can be halogen, C₁-C₂-haloalkyl, H, C₁-C₆-alkyl, C₁-C₆-alkyl sulfide, OH, O—(C₁-C₆-alkyl), —O—(C₂-C₆-alkyl)-N(R^{x})₂, or —O—(C₂-C₆-alkyl)-N(R^{x})₃⁺halogen⁻; R⁶ of formula (II) can be halogen, C₁-C₂-haloalkyl, H, C₁-C₆-alkyl, —S—(C₁-C₆-alkyl), OH, O—(C₁-C₆-alkyl), —O—(C₂-C₆-alkyl)-N(R⁵)₂, or —O—(C₂-C₆-alkyl)-N(R⁵)₃⁺halogen⁻; R^{α} is H, halogen, or C₁-C₆-alkyl; R^{β} of formula (II) can be OH, O—(C₁-C₆-alkyl), —O—(C₂-C₆-alkyl)-N(R⁵)₂, or —O—(C₂-C₆-alkyl)-N(R^{x})₃⁺halogen⁻; R^{N} of formula (II) can be halogen, C₁-C₂-haloalkyl, H, C₁-C₆-alkyl, C₁-C₆-alkyl sulfide, OH, O—(C₁-C₆-alkyl), —O—(C₂-C₆-alkyl)-N(R^{x})₂, or —O—(C₂-C₆-alkyl)-N(R^{x})₃⁺halogen⁻; and R^{x} is independently H or C₁-C₄-alkyl.

In embodiments, the composition comprises a compound having the following chemical formula (I):

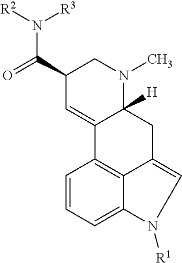

where the non-limiting exemplary values of the R groups in the above substituted chemical structure are represented in the following table (Table 2):

| Name | R¹ | R² | R³ |
|---|---|---|---|
| LSD | H | CH₂CH₃ | CH₂CH₃ |
| Ergine | H | H | H |
| R-2-butyl | H | H | CH(CH₃)CH₂CH₃ |
| R-2-pentylamine | H | H | CH(CH₃)CH₂CH₂CH₃ |
| Analog of ergoline | H | C₂H₅ | H |
| Analog of ergoline | H | H | C₂H₅ |
| LSD | H | C₂H₅ | C₂H₅ |
| Analog of ergoline | H | C₂H₅ | CH₂CH₂CH₃ |
| Analog of ergoline | H | C₂H₅ | CH(CH₃)₂ |
| Analog of ergoline | H | CH₂CH₂CH₃ | H |
| Analog of ergoline | H | H | CH₂CH₂CH₃ |
| Analog of ergoline | H | CH₂CH₂CH₃ | CH₂CH₂CH₃ |
| Analog of ergoline | H | CH₂CH₂CH₃ | C₂H₅ |
| Analog of ergoline | H | CH₂CH₂CH₃ | CH(CH₃)₂ |
| Analog of ergoline | H | CH(CH₃)₂ | H |
| Analog of ergoline | H | H | CH(CH₃)₂ |
| Analog of ergoline | H | CH(CH₃)₂ | CH(CH₃)₂ |
| Analog of ergoline | H | CH(CH₃)₂ | C₂H₅ |
| Analog of ergoline | H | CH(CH₃)₂ | CH₂CH₂CH₃ |

In some embodiments, R¹ of formula (I) can be H, C₁-C₆-alkyl, OH, O—(C₁-C₆-alkyl), halogen, or C₁-C₄-haloalkyl; R² of formula (I) can be H, C₁-C₆-alkyl, OH, O—(C₁-C₆-alkyl), halogen, or C₁-C₄-haloalkyl; and R³ of formula (I) can be H, C₁-C₆-alkyl, OH, O—(C₁-C₆-alkyl), halogen, or C₁-C₄-haloalkyl.

In embodiments, the composition comprises a compound having the following chemical formula (III):

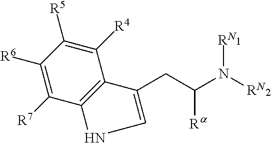

where the non-limiting exemplary values of the R groups in the above substituted chemical structure are represented in the following table (Table 3):

| Name | R^{N}₁ | R^{N}₂ | R^{α} | R⁴ | R⁵ | R⁶ | R⁷ |
|---|---|---|---|---|---|---|---|
| 6-fluoro-psilocin | C | C | H | OH | H | F | H |
| 7-fluoro-psilocin | C | C | H | OH | H | H | F |
| 4-fluoro-5- | C | C | H | F | OCH₃ | H | H |
| methoxy-DMT | | | | | | | |
| 6-fluoro-5- | C | C | H | H | OCH₃ | F | H |
| methoxy-DMT | | | | | | | |
| □-Methyl- | H | H | CH₃ | H | H | H | H |
| tryptamine | | | | | | | |
| Serotonin | H | H | H | H | OH | H | H |
| 5-methoxy-DMT | C | C | H | H | OCH₃ | H | H |
| N,N- | C | C | H | H | H | H | H |
| dimethyltryptamine | | | | | | | |

In some embodiments, R^{N1} of formula (III) can be H, C₁-C₆-alkyl, OH, O—(C₁-C₆-alkyl), halogen, or C₁-C₄-haloalkyl; R^{N2} of formula (III) can be H, C₁-C₆-alkyl, OH, O—(C₁-C₆-alkyl), halogen, or C₁-C₄-haloalkyl; R^{α} of formula (I) can be H, C₁-C₆-alkyl, OH, O—(C₁-C₆-alkyl), halogen, or C₁-C₄-haloalkyl; R⁴ of formula (I) can be H, C₁-C₆-alkyl, OH, O—(C₁-C₆-alkyl), halogen, or C₁-C₄-haloalkyl; R⁵ of formula (I) can be H, C₁-C₆-alkyl, OH, O—(C₁-C₆-alkyl), halogen, or C₁-C₄-haloalkyl; R⁶ of formula (I) can be H, C₁-C₆-alkyl, OH, O—(C₁-C₆-alkyl), halogen, or C₁-C₄-haloalkyl; and R⁷ of formula (I) can be H, C₁-C₆-alkyl, OH, O—(C₁-C₆-alkyl), halogen, or C₁-C₄-haloalkyl.

In some embodiments, a compound of the invention (for example a compound of formula (I), (II), or (III)) binds to a serotonin receptor in a subject. Non-limiting examples of serotonin receptors include HTR2A (5-hydroxytryptamine receptor 2A isoform 1 (GenBank Accession No. for nucleotide sequence: NM_000621.4 and GenBank Accession No. for amino acid sequence: NP_000612.1); 5-hydroxytryptamine receptor 2A isoform 2 (GenBank Accession No. for nucleotide sequence: NM_001165947.2 and GenBank Accession No. for amino acid sequence: NP_001159419.1)); HTR2B (5-hydroxytryptamine receptor 2B isoform 1 (GenBank Accession No. for nucleotide sequence: NM_000867.4 and GenBank Accession No. for amino acid sequence: NP_000858.3); 5-hydroxytryptamine receptor 2B isoform 2 (GenBank Accession No. for nucleotide sequence: NM_001320758.1 and GenBank Accession No. for amino acid sequence: NP_001307687.1)); and HTR2C (5-hydroxytryptamine receptor 2C isoform a precursor (GenBank Accession No. for nucleotide sequence: NM_000868.3 and GenBank Accession No. for amino acid sequence: NP_000859.1); 5-hydroxytryptamine receptor 2C isoform a precursor (GenBank Accession No. for nucleotide sequence: NM_001256760.2 and GenBank Accession No. for amino acid sequence: NP_001243689.1); 5-hydroxytryptamine receptor 2C isoform b precursor (GenBank Accession No. for nucleotide sequence: NM_001256761.2 and GenBank Accession No. for amino acid sequence: NP_001243690.1)).

In some embodiments, the serotonin receptor comprises SEQ ID NO: 1 (amino acids 1-481 having GenBank Accession No. NP_000858.3):

MALSYRVSELQSTIPEHILQSTFVHVISSNWSGLQTESIPEEMKQIVEEQ
GNKLHWAALLILMVIIPTIGGNTLVILAVSLEKKLQYATNYFLMSLAVAD
LLVGLFVMPIALLTIMFEAMWPLPLVLCPAWLFLDVLFSTASIMHLCAIS
VDRYIAIKKPIQANQYNSRATAFIKITVVWLISIGIAIPVPIKGIETDVD
NPNNITCVLTKERFGDFMLFGSLAAFFTPLAIMIVTYFLTIHALQKKAYL
VKNKPPQRLTWLTVSTVFQRDETPCSSPEKVAMLDGSRKDKALPNSGDET
LMRRTSTIGKKSVQTISNEQRASKVLGIVFFLFLLMWCPFFITNITLVLC
DSCNQTTLQMLLEIFVWIGYVSSGVNPLVYTLFNKTFRDAFGRYITCNYR
ATKSVKTLRKRSSKIYFRNPMAENSKFFKKHGIRNGINPAMYQSPMRLRS
STIQSSSIILLDTLLLTENEGDKTEEQVSYV

In some embodiments, the serotonin receptor comprises SEQ ID NO: 2 (amino acids 1-471 having GenBank Accession No. NP_000612.1):

MDILCEENTSLSSTTNSLMQLNDDTRLYSNDFNSGEANTSDAFNWTVDSE
NRTNLSCEGCLSPSCLSLLHLQEKNWSALLTAVVIILTIAGNILVIMAVS
LEKKLQNATNYFLMSLAIADMLLGFLVMPVSMLTILYGYRWPLPSKLCAV
WIYLDVLFSTASIMHLCAISLDRYVAIQNPIHHSRFNSRTKAFLKIIAVW
TISVGISMPIPVFGLQDDSKVFKEGSCLLADDNFVLIGSFVSFFIPLTIM
VITYFLTIKSLQKEATLCVSDLGTRAKLASFSFLPQSSLSSEKLFQRSIH
REPGSYTGRRTMQSISNEQKACKVLGIVFFLFVVMWCPFFITNIMAVICK
ESCNEDVIGALLNVFVWIGYLSSAVNPLVYTLFNKTYRSAFSRYIQCQYK
ENKKPLQLILVNTIPALAYKSSQLQMGQKKNSKQDAKTTDNDCSMVALGK
QHSEEASKDNSDGVNEKVSCV

In some embodiments, the serotonin receptor comprises SEQ ID NO: 3 (amino acids 1-458 having GenBank Accession No. NP_000859.1):

MVNLRNAVHSFLVHLIGLLVWQCDISVSPVAAIVTDIFNTSDGGRFKFPD
GVQNWPALSIVIIIIMTIGGNILVIMAVSMEKKLHNATNYFLMSLAIADM
LVGLLVMPLSLLAILYDYVWPLPRYLCPVWISLDVLFSTASIMHLCAISL
DRYVAIRNPIEHSRFNSRTKAIMKIAIVWAISIGVSVPIPVIGLRDEEKV
FVNNTTCVLNDPNFVLIGSFVAFFIPLTIMVITYCLTIYVLRRQALMLLH
GHTEEPPGLSLDFLKCCKRNTAEEENSANPNQDQNARRRKKKERRPRGTM
QAINNERKASKVLGIVFFVFLIMWCPFFITNILSVLCEKSCNQKLMEKLL
NVFVWIGYVCSGINPLVYTLFNKIYRRAFSNYLRCNYKVEKKPPVRQIPR
VAATALSGRELNVNIYRHTNEPVIEKASDNEPGIEMQVENLELPVNPSSV
VSERISSV

In some embodiments, the compound of the invention can bind to amino acid residue(s) of a serotonin receptor comprising position(s) 113, 114, 118, 131, 132, 133, 135, 136, 139, 140, 190, 203, 207, 209, 213, 214, 217, 218, 221, 222, 225, 242, 293, 308, 336, 337, 339, 340, 341, 343, 344, 362, 363, 366, 367, or a combination thereof, of SEQ ID NOS: 1, 2, or 3.

In some embodiments, the compound of the invention can bind to amino acid residues T114, W131, L132, D135, V136, S139, T140, V190, L209, F214, F217, M218, G221, S222, A225, H242, W337, F340, F341, N344, L362, E363, V366, or a combination thereof, of SEQ ID NO: 1.

In some embodiments, the compound of the invention can bind to amino acid residues M114, S131, L133, 1135, L136, Y139, R140, T190, S203, S207, P209, F213, D217, D218, V221, F222, G225, S242, W336, F339, F340, N343, L362, N363, V366, or a combination thereof, of SEQ ID NO: 2.

Embodiments as described herein can be administered to a subject as a prodrug. A prodrug is a medication or compound that, after administration, is metabolized into a pharmaceutically active drug. Inactive prodrugs are pharmacologically inactive medications or compounds that are metabolized into an active form within the body.

Following successful completion of animal trials using common mammals, (R)-DOI and other 5-HT2A agonists will be tested in human patients with symptoms or diseases of enhanced immunological response in clinical trials conducted in compliance with applicable laws and regulations.

Specific 5-HT2A agonists used in the invention can be administered to a patient by any suitable means, including oral, intravenous, parenteral, subcutaneous, intrapulmonary, topical, intravitreal, dermal, transmucosal, rectal, and intranasal administration. Parenteral infusions include intramuscular, intravenous, intraarterial, or intraperitoneal administration. The compounds can also be administered transdermally, for example in the form of a slow-release subcutaneous implant or as a transdermal patch. They can also be administered by inhalation. Although direct oral administration can cause some loss of beneficial or desired activity, for example anti-inflammatory activity, the agonists can be packaged in such a way to protect the active ingredient(s) from digestion by use of enteric coatings, capsules or other methods known in the art.

For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

The compound of formula (I), (II), or (III), or the composition comprising a compound of formula (I), (II), or (III) can be administered to the subject one time (e.g., as a single injection or deposition). Alternatively, administration can be once or twice daily to a subject in need thereof for a period of from about 2 to about 28 days, or from about 7 to about 10 days, or from about 7 to about 15 days. It can also be administered once or twice daily to a subject for a period of 1, 2, 3, 4, 5, 6, 7, 8, 9, 10, 11, 12, 13, 14, 15, 16, 17, 18, 19, 20, 21, 22, 23, 24 times per year, or a combination thereof.

The dosage can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion.

A therapeutically effective dose can depend upon a number of factors known to those of ordinary skill in the art. The dose(s) can vary, for example, depending upon the identity, size, and condition of the subject or sample being treated, further depending upon the route by which the composition is to be administered, if applicable, and the effect which the practitioner desires. These amounts can be readily determined by the skilled artisan.

In some embodiments, the therapeutically effective amount of a compound of the invention administered to a subject is at least about 0.0001 mg/kg body weight, 0.0005 mg/kg body weight, 0.001 mg/kg body weight, 0.005 mg/kg body weight, 0.01 mg/kg body weight, 0.05 mg/kg body weight, 0.1 mg/kg body weight, at least about 0.25 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight.

Any of the therapeutic applications described herein can be applied to any subject in need of such therapy, including, for example, a mammal such as a mouse, a rat, a dog, a cat, a cow, a horse, a rabbit, a monkey, a pig, a sheep, a goat, or a human. In some embodiments, the subject is a mouse, rat, pig, or human. In some embodiments, the subject is a mouse. In some embodiments, the subject is a rat. In some embodiments, the subject is a pig. In some embodiments, the subject is a human.

Compounds of formula (I), (II), or (III) can be incorporated into pharmaceutical compositions suitable for administration. Such compositions can comprise a compound of formula (I), (II), or (III) and a pharmaceutically acceptable carrier. Thus, in some embodiments, the compounds of the invention are present in a pharmaceutical composition.

In embodiments, the agonist is not DOI.

Compositions

The term “composition” can refer to a single compound, or can refer to a combination of at least two compounds. For example, a composition can comprise a serotonin receptor agonist and a pharmaceutically acceptable carrier. In other embodiments, the composition can comprise more than two compounds. For example, a composition can comprise a serotonin receptor agonist, a pharmaceutically acceptable carrier, and an antipathogenic agent.

Pharmaceutically acceptable carrier preparations include sterile, aqueous or non-aqueous solutions, suspensions, and emulsions. Examples of non-aqueous solvents are propylene glycol, polyethylene glycol, carboymethylcellulose, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. The active therapeutic ingredient can be mixed with excipients that are pharmaceutically acceptable and are compatible with the active ingredient. Suitable excipients include water, saline, dextrose, glycerol and ethanol, or combinations thereof. Intravenous vehicles include fluid and nutrient replenishers, electrolyte replenishers, such as those based on Ringer's dextrose, and the like. Preservatives and other additives can also be present such as, for example, antimicrobials, anti-oxidants, chelating agents, inert gases, and the like.

The form can vary depending upon the route of administration. For example, compositions for injection can be provided in the form of an ampoule, each containing a unit dose amount, or in the form of a container containing multiple doses. In some embodiments, parenteral preparations can be enclosed in ampoules, disposable syringes or multiple dose vials made of glass or plastic.

In some embodiments, pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In embodiments, the composition is sterile and is fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

In some embodiments, sterile injectable solutions can be prepared by incorporating the compound in the required amount in an appropriate solvent with one or a combination of ingredients enumerated herein, as required, followed by filtered sterilization. Dispersions can be prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional ingredient from a previously sterile-filtered solution thereof.

In some embodiments, oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

A compound in accordance with the invention can be formulated into therapeutic compositions as pharmaceutically acceptable salts, for example a hydrochloride salt (e.g., the (R)-DOI used in the examples herein). These salts include acid addition salts formed with inorganic acids, for example hydrochloric or phosphoric acid, or organic acids such as acetic, oxalic, or tartaric acid, and the like. Salts also include those formed from inorganic bases such as, for example, sodium, potassium, ammonium, calcium or ferric hydroxides, and organic bases such as isopropylamine, trimethylamine, histidine, procaine and the like.

A method for controlling the duration of action comprises incorporating the active compound into particles of a polymeric substance such as a polyester, peptide, hydrogel, polylactide/glycolide copolymer, or ethylenevinylacetate copolymers. Alternatively, an active compound can be encapsulated in microcapsules prepared, for example, by coacervation techniques or by interfacial polymerization, for example, by the use of hydroxymethylcellulose or gelatin-microcapsules or poly(methylmethacrylate) microcapsules, respectively, or in a colloid drug delivery system. Colloidal dispersion systems include macromolecule complexes, nanocapsules, microspheres, beads, and lipid-based systems including oil-in-water emulsions, micelles, mixed micelles, and liposomes.

Embodiments, such as those suitable for ocular uses, incorporate additives to increase dispersion of the drugs in the eye while also increasing retention in the eye. Non-limiting examples of such additives comprise carboxymethylcellulose or polyethylene glycol.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art.

In embodiments, the serotonin receptor agonist can be administered to a subject in a composition comprising at least one additional bioactive agent. Non-limiting examples of bioactive agents comprise an antimicrobial agent, an anti-pathogenic agent, a drug, or a combination thereof. Non-limiting examples of antimicrobial agents comprise an antiviral agent, an antibacterial agent, an antifungal agent, an antiprotozoal agent, or a combination thereof.

An “anti-pathongeic agent” can refer to an agent, such as a chemical agent or a biologic, that inhibits activity or prevents the production and/or release of a pathogenic factor which leads to the destructive effects of tissues, such as at the site of an infection.

In embodiments, non-limiting examples of an antiviral agent comprise TFT, Acyclovir, gancyclovir, penciclovir, famiciclovir, cidofovir and its analog derivatives; ribavirin, interferon, phosphonoacetate, Foscarnet, Valacyclovir, and Valgancyclovir. TFT and ganciclovir, for example, are relevant for the infections of the eye, such as in herpetic infections.

In embodiments, non-limiting examples of an antibacterial agent comprise aminoglycosides, fluoroquinolones, beta-lactams, macrolide, and tetracyclines.

In embodiments, non-limiting examples of an antifungal agent comprise at least one polyene, at least one azole, at least one allylamine, echinocardins or a combination thereof.

In embodiments, non-limiting examples of antiprotozoal agents comprise chloroquine, pyrimethamine, mefloquine, hydroxychloroquine, metronidazole, atovaquone, or a combination thereof.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

We have begun to demonstrate that agonists of the serotonin receptor pathway, such as DOI, can be delievered systemically or through a composed topical ocular drop in order to prevent and resolve pathogen-elicted host-mediated disease processes. This formulation can or can not require inclusion of secondary compounds that prevent viral replicative processes, and this requirement appears to be preliminarily dependent on the genetics of the host. We have initially run ocular topical treatment studies comparing different topical ocular compositions and demonstrated that in a herpetic disease model, inclusion of DOI can effectively suppres acute and chronic herpes-associated eye disease. Importantly, in a herpetic eye disease model this advance is superior at controlling both acute and chronic vision-threatening disease when compared to the gold-standard anti-herpetic TFT. Specifically, treatment with compositions that included DOI suppressed inflammation-associated disease processes including neovascularization of the cornea, trafficking of inflammatory cells into the cornea, and epithelial and stromal damage. These compositions can or cannot require additional inclusion of compounds that suppress pathogen replicative processes

Non-limiting examples of embodiments comprise treatment of severe viral eye diseases and prevention/resolution of chronic ocular disease progression; prevention and/or treatment of ocular hypersensitivity processes that result in hypersensitivity-associated ocular diseases; prevention of pathological ocular neovascularization and angiogenesis as a primary indication or as associated with other ocular hypersensitivity sequelae mediated by genetics or infection, for example; hypersensitivity or, for example; prevention of herpetic and stromal keratitis; prevention of Adenoviral conjunctivitis; hypersensitivity; or hypersensitivity. Despite the availability of effective anti-infectives that can suppress replication of specific pathogens, pathogen-mediated initiation of hypersensitivity hypersensitivity-associated processes causes severe disease presentation that can become a chronic self-perpetuating process. In the eye, accumulation of hypersensitivity hypersensitivity-mediated processes can result in severe disease presentations that are independent of the replication of the pathogen. Therefore, effective treatment and resolution of the pathogen by current anti-infectives can not in of itself prevent chronic hypersensitivity-associated disease processes that ultimately damage ocular tissues. This includes: cell-mediated hypersensitivity of these tissues, destruction of host tissues by hypersensitivity processes, cytokine mediated hypersensitivity and disease, and pathological vascularization of the tissue. Treatment with serotonin-agonists such as DOI, is a new approach that can suppress these host mediated disease processes without some of the potential side effects that are associated with classical immunosuppressives. Our data indicates that compounds such as DOI can effectively prevent acute and chronic herpetic eye disease that normally results in severe irreversible hypersensitivity-associated destruction of the cornea and blindness. DOI also prevented pathological vascularization of the normally avascular cornea—a process that contributes to several eye-associated disease processes. Drugs that prevent pathogen replication fail to control these inflammation-mediated processes and as such disease progresses irrespective of a drug's ability to control pathogen replication. Therefore, use of compounds that target this pathway alone, or in combination with other antiinfectives, can be an effective means in preventing the longterm chronic consequences of pathogen infection and associated acute and chronic disease processes.

Non-limiting examples of future studies comprise studies to optimize dosing and compositions; repeating animal model studies in different genetic backgrounds that exhibit different inflammatory or pathological disease presentations, and assessing if DOI can be utilized on its own or if depending on genetic background it requires co-administration with an antiviral; examining additional hypersensitivity viral models of disease, including adenovirus ocular infections and respiratory viral infections; determining what effects does treatment have on levels of viral replication; and cell-type drug response and toxicity studies.

### Example 2

For Study with C57bl/6 Mice:

C57bl/6 mice, which can respond with a TH1 biased immune response, were randomly sorted into 3 treatment arms: 1) Ophthalmic Balanced Saline Solution (BSS) treated; 2) DOI treated (XTPFDOI); 3) 0.5% TFT with DOI (TFT+XTPFDOI). Animals were anesthetized with xylene:ketamine and both eyes were scarified in a cross hatch pattern using a curved needle. Immediately following ocular scarification, eyes were inoculated with a 3 microliter drop containing 12,000 plaque forming units (PFU) of Herpes Simplex Virus type 1 (HSV-1) RE strain. The next morning following infection animals were treated with the respective treatment as assigned within their treatment arm. Treatments were applied topically to the eye in a 4 microliter drop. Drops were applied 4× daily from 9 am to 5:30 pm starting immediately following clinical scoring. Treatments were applied for the first 8 days post infection and then stopped on day 8. Clinical scoring was done using a slit lamp biomicroscope magnified at 16× on the days indicated by a single individual masked to the drug treatment parameters. Slit lamp biomicroscopy also included fluorescein exclusion labeling of the corneal surface following scoring of all clinical parameters. Each eye was scored independently.

For Study with BALB/c Mice:

16 BALB/c mice, which can respond with a TH2 biased immune response, were randomly sorted into 3 treatment arms: 1) Ophthalmic Balanced Saline Solution diluted 1:1 in PBS (BSS+PBS) treated (6 mice); 2) DOI treated (XTPFDOI) (5 mice); 3) 1.0% TFT (TFT+PBS) (5 mice). Animals were anesthetized with xylene:ketamine and both eyes were scarified in a cross hatch pattern using a curved needle. Immediately following ocular scarification, eyes were inoculated with a 3 microliter drop containing 10,000 plaque forming units (PFU) of Herpes Simplex Virus type 1 (HSV-1) RE strain. The next morning following infection animals were treated with the respective treatment as assigned within their treatment arm. Treatments were applied topically to the eye in a 4 microliter drop. Drops were applied 4× daily from 9 am to 5:30 pm starting immediately following clinical scoring. Treatments were applied for the first 8 days post infection and then stopped on day 8. Clinical scoring was done using a slit lamp biomicroscope magnified at 16× on the days indicated by a single individual masked to the drug treatment parameters. Slit lamp biomicroscopy also included fluorescein exclusion labeling of the corneal surface following scoring of all clinical parameters. Each eye was scored independently. Clinically clear eyes were scored as such if no apparent signs of disease were present in any clinical parameter during the chronic phase.

At day 15 post infection, a stage that will be during chronic immune-associated disease with no virus present, animals were euthanized and the eyes were removed for histology. Random representative eyes were prepared by taking sections through the central cornea and processed by H&E histology for visualization. Sections were examined microscopically and photographed across the central cornea. Multiple eyes from each group that showed the best representation of that group's clinical scores, extremes, and midpoints are shown.

### Example 3

Project Summary:

Our and our collaborator's initial findings indicate that in ocular models of disease, DOI potently inhibits disease-associated vascularization of these tissues, thereby preventing the chronic pathology normally associated with disease progression. The mechanisms by which DOI accomplishes this suppression has not been elucidated. Without wishing to be bound by theory, DOI can modulate vasculogenesis and vascular homeostasis in these disease processes through direct effects on vascular cells and suppression of chronic inflammatory processes.

A representative pathological vascularization-associated ocular model system of herpetic stromal keratitis can be used to evaluate the effects of DOI. This animal model system is complemented by established in vitro mechanistic studies to assess the direct effects of DOI on vascular cell biology and function. The contributions of 5-HT receptors in this disease process has not previously been explored. Without being bound by theory, foundational data from this study can allow for development of DOI as a therapeutic for suppression of vascularization-associated ocular disease processes.

### INTRODUCTION

Physiological angiogenesis and neovascularization are required for embryonic development1, tissue remodeling and wound healing2,3. However, in certain tissues and diseases, dysregulation of these tightly controlled processes can result in vascularization-mediated pathological conditions3,4,5. Pathological vascularization and dysregulation of vascular function are critical determinates in the outcomes of including ocular neovascularization diseases 10,11, including blinding stromal keratitis, proliferative retinopathies 11, and macular degeneration. Recently, a role in modulating inflammatory processes was discovered for the serotonin receptor family, also known as the 5-hydroxytyptamine receptors (5-HT)13,14. Once thought to only be involved in modulating release of neurotransmitters in the central and peripheral nervous system, these GPCRs are finding new life as modulators of broad biological functions, including in cardiovascular biology and as immune regulators. As shown herein, activation of the 5-HT2a receptor with the agonist DOI can effectively suppress vascularization-associated processes by inhibiting TNFα activity 15,16,17. As discussed herein, these drugs will be used to generate data through the following:

To Evaluate the Therapeutic Efficacy of 5-HT Receptor Modulation for Amelioration of Pathological Vascularization- and Inflammation-Associated Diseases.

Data demonstrates that the 5-HT agonist, R-DOI can suppress disease processes within the eye. Specifically, our ophthalmic formulation of R-DOI suppressed HSV-induced pathological vascularization of the eye and abolished chronic host-mediated vision-threatening disease processes. Without wishing to be bound by theory, this indicates that 5-HT receptors participate in the associated disease processes within ocular tissues and that modulation of specific 5-HT receptor activities has therapeutic potential for prevention and resolution of ocular disease.

Therapeutic Potential of Modulating 5-HT Receptor Activity for Treatment of Pathological Ocular Vascularization- and Inflammation-Associated Ocular Diseases Will be Evaluated:

Part 1A: In an Ocular Model of Chronic Herpetic Stromal Keratitis, in which the Extent of Vascularization of the Eye is a Prognostic Indicator of Vision-Threatening Pathology.

Significance

Herpetic Stromal Keratitis

Globally, infection- and inflammation-associated eye diseases are the leading causes of corneal blindness and visual morbidity, with over 500 million individuals affected18. The model ocular viral pathogen in these studies, Herpes Simplex virus type I (HSV-1), is present in 70-90% of the population and is the leading cause of corneal blindness in developed countries 19,20. The National Eye Institute estimates that 450,000 Americans have experienced some form of ocular herpetic disease, with 50,000 new and recurrent cases diagnosed19. Current anti-pathogen drugs fail to inhibit pathogen-induced inflammatory responses21,23. As such, approximately 25% of cases present with serious inflammation-associated stromal keratitis. Individuals that have experienced ocular herpes, have a 50% chance of recurrence19. Each repeated episode triggers a chronic inflammatory disease process that can result in vascularization and subsequent vision threatening scarring of the cornea that eventually requires corneal transplantation to resolve21,26. Immuno-suppressive drugs, such as dexamethasone, can control deleterious inflammation; however, they also license uncontrolled pathogen replication and are associated with loss of an intact corneal epithelial barrier, increased ocular pressure and eventual deterioration of vision27,28. By contrast, modulation of 5-HT receptor activity within the eye has been shown to decrease ophthalmic pressure29. Combined with its newly discovered anti-vascularization properties, its potential within the eye can be immense, for example by replacing corticosteroids for several ocular disease indications.

Pathological vascularization and dysregulation of vascular function are main contributors to all infectious and many non-infectious disease processes of the eye. The association of drug targetable 5-HT processes for ocular diseases represents a new and innovative approach.

Research Strategy and Initial Results

Overall Approach

There are a large number and varying functions of the 5-HT receptors, as well as the differing effects that they induce within different cell-types. Developing an understanding of how mechanistically specific 5-HT receptors function to both control and induce disease represents a significant gap of knowledge within the field that is ripe for exploration. Our overall approach is to evaluate the therapeutic potential of modulation of 5-HT receptors in ocular disease processes. We will compare the effects of various 5-HT receptor agonists and antagonists evaluating whether treatment enhances or suppresses presentation of clinical disease parameters and then focus these studies on drugs that can improve disease outcomes. By exploring both agonists and antagonists of these receptors the data obtained from these studies will also provide critical foundational evidence of how specific 5-HT receptors can contribute mechanistically to disease outcomes, whether positive or negative. This will establish a mechanistic role for 5-HT receptor function in these diseases, fill a significant gap of knowledge in the field, and help settle the controversies associated with the bifunctional nature of these receptors in disease. Most importantly, it will provide initial data and allow for examination of these outcomes utilizing more basic science mechanistic models, such as receptor-specific knockout mice.

Overall Analysis

Therapeutic efficacy will be assessed by: 1) Clinical scoring as outlined in disease specific models; 2) histopathological findings; 3) Supportive immunological data, including sera inflammatory cytokine levels and infiltration of cells within effected tissues. Histopathology will be performed within an imaging and histology core, with pathological assessment scoring and advice subsequently provided. All samples will be imaged and analyzed using equipment in the imaging core. FACS and cytokine analysis will be performed in coordination with the cell and molecular analysis core. Statistical analysis for all clinical parameters will be done as previously in conjunction with the biostatistics core.

Part 1A: Evaluation of Therapeutic Efficacy of 5-HT Receptor Modulation in an Ocular Model of Chronic Herpetic Stromal Keratitis.

Rationale and Initial Results:

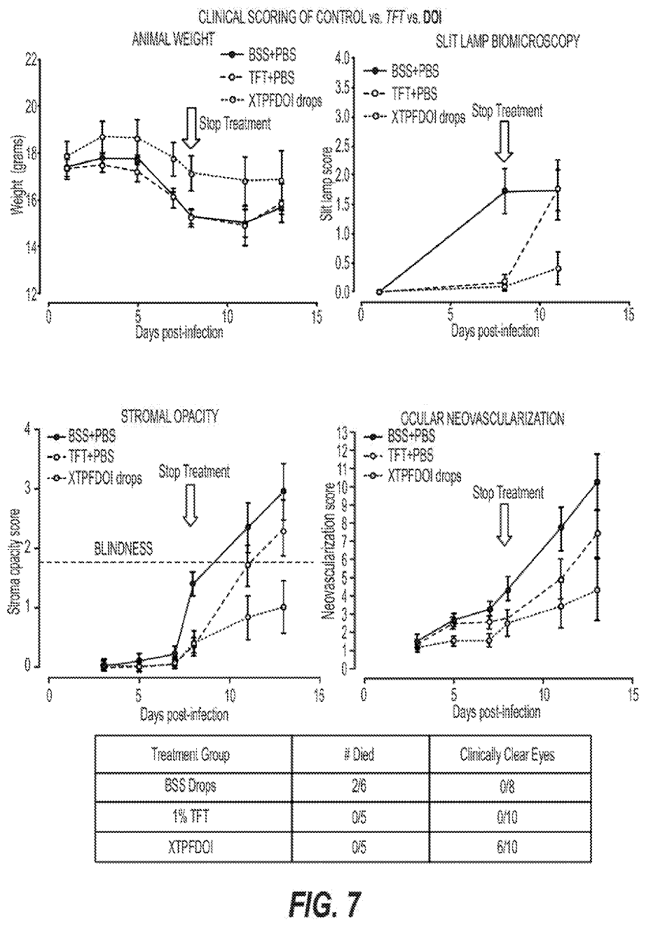

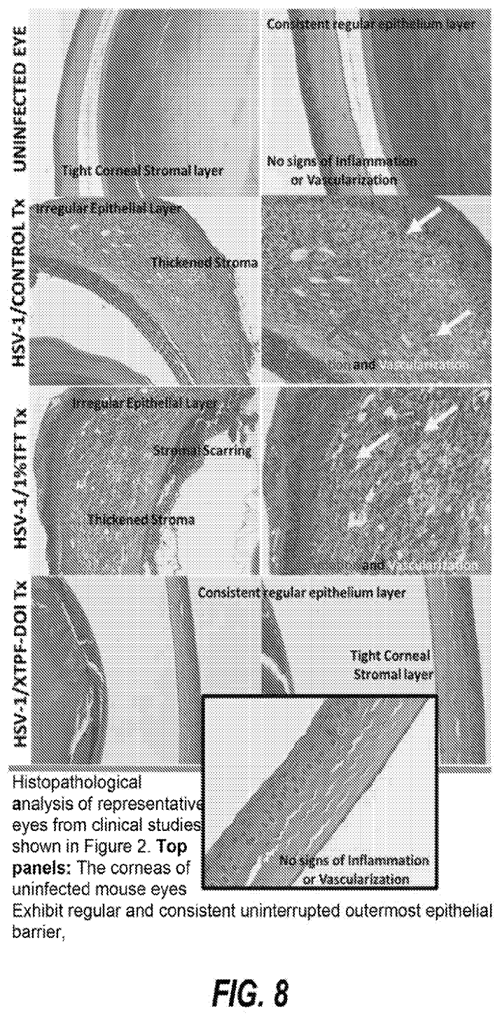

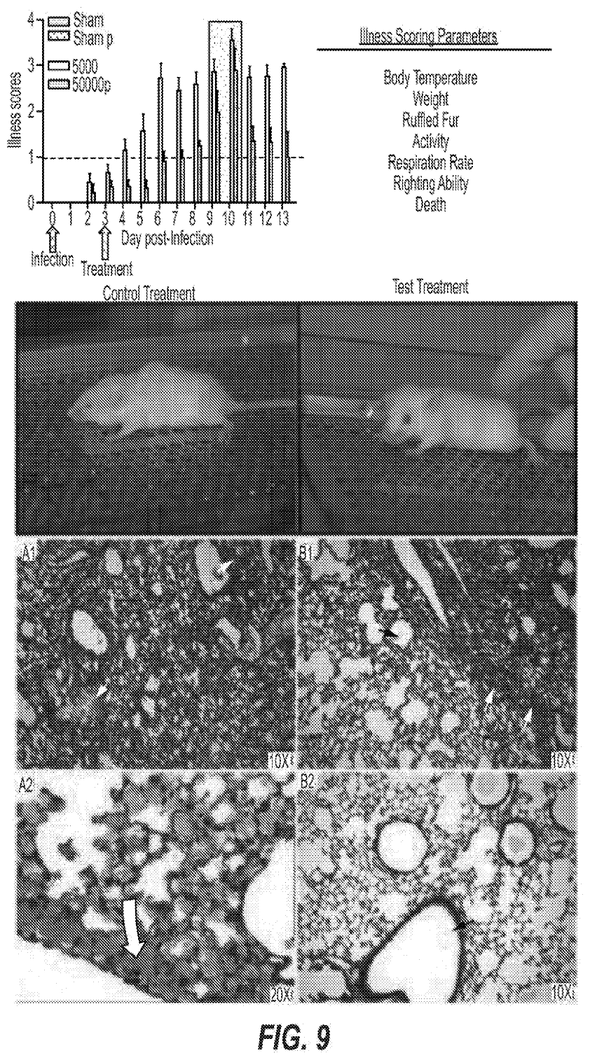

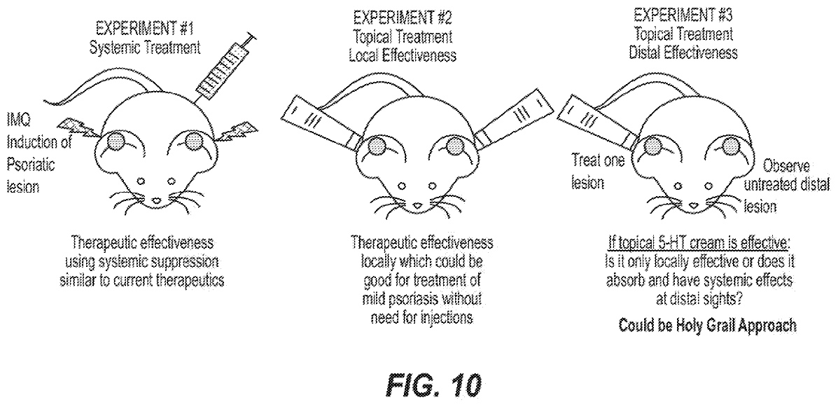

5-HT receptors play a role in ocular function, regulation of vascular integrity, and ocular pressure. Without being bound by theory, the 5-HT agonist DOI can suppress inflammatory responses. In one embodiment, these responses can be the main initiator of viral mediated ocular disease processes. Thus, an ophthalmic topical formulation of DOI (here, XTPF-DOI) was developed and its ability to inhibit the long-term inflammatory- and vascularization-mediated disease processes that are responsible for inducing corneal blindness following HSV infection was assessed. Data from a mouse model of HSK demonstrated that DOI was effective at suppressing HSV-associated ocular disease sequelae and progression to blindness (FIG. 7 and FIG. 8).

Experimental Design & Methods-Mouse Model of Herpetic Stromal Keratitis

We have generated data for the effectiveness of 5-HT receptor agonists in prevention and resolution of HSK within the mouse model. The data has been repeated, and additional replicates, virological data and better quantitative histopathological examination are planned. Therefore, experiments will be repeated as described in FIG. 7 and FIG. 8 collecting additional supportive and publishable clinical disease, virological and histopathological data. We will also evaluate and pathologically score each eye's histology section.

ED&M-Rabbit Model of Herpetic Stromal Keratitis

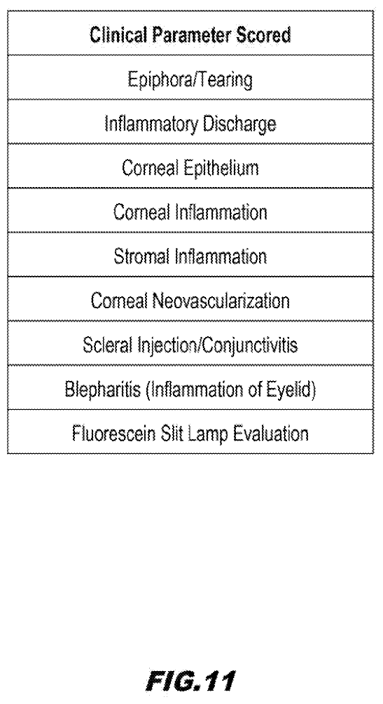

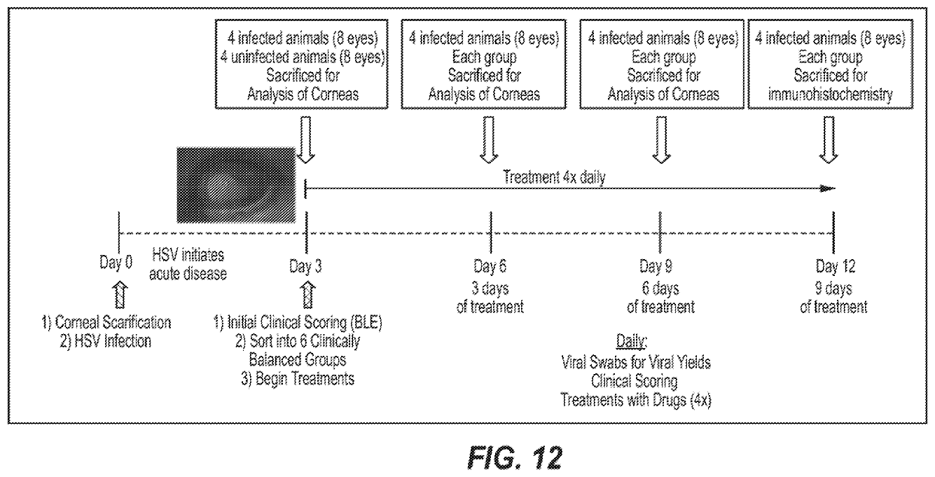

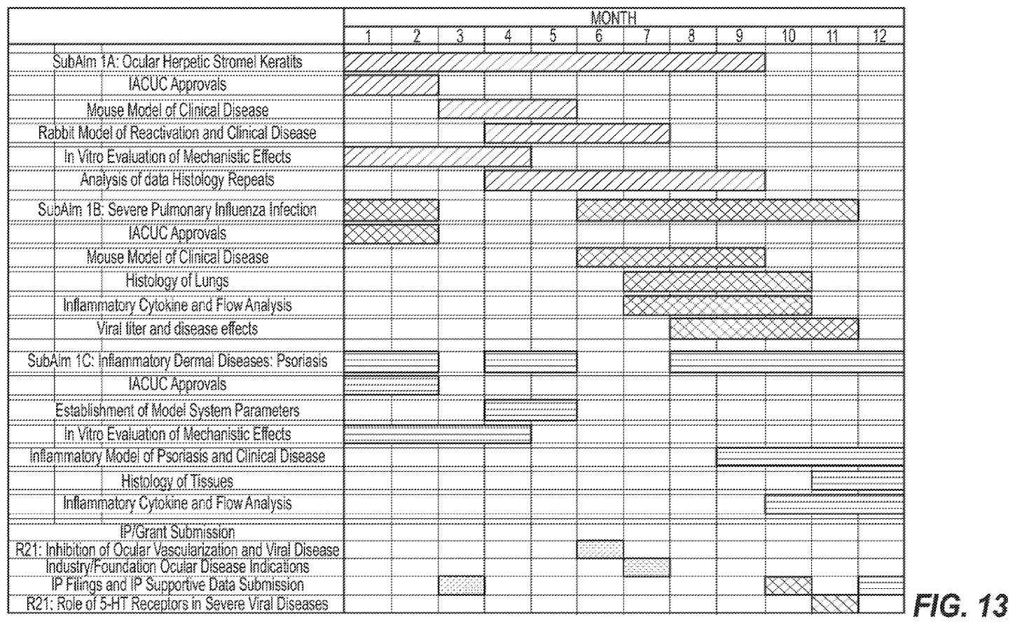

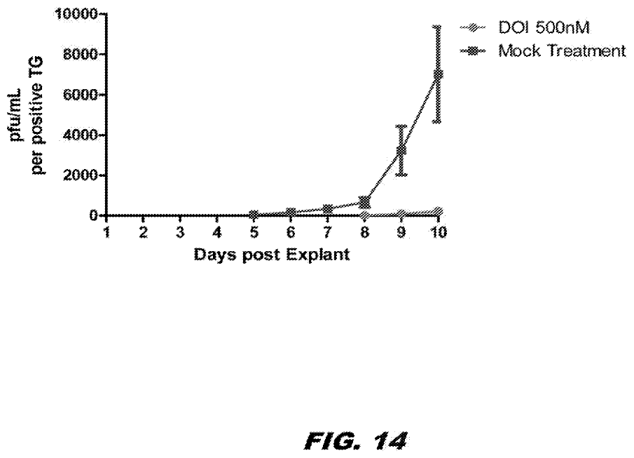

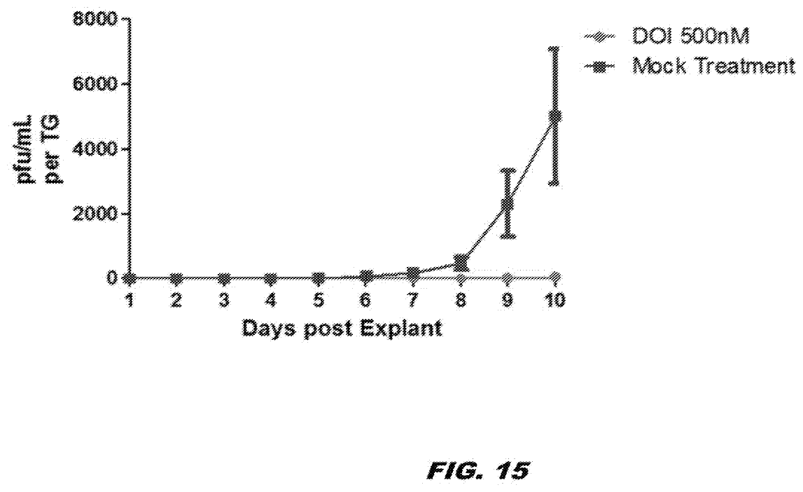

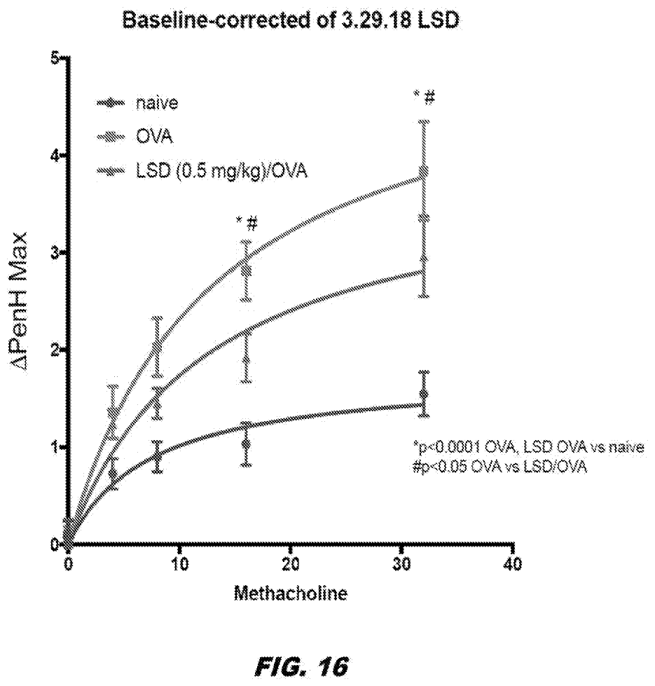

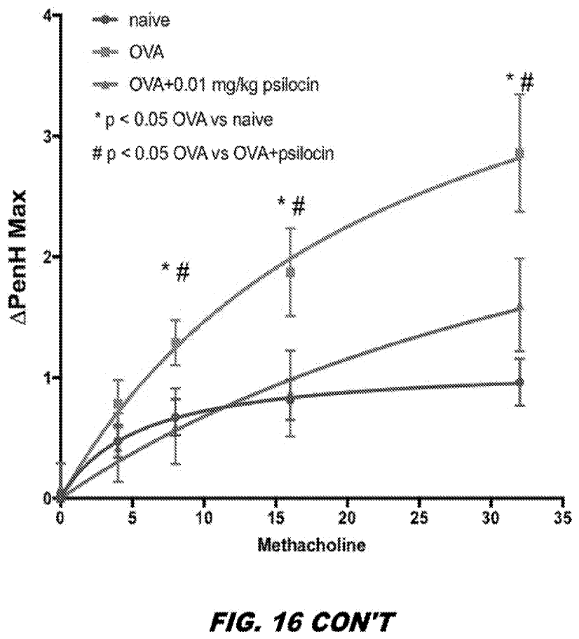

The rabbit eye is an FDA accepted ocular preclinical model that accurately reflects clinical disease parameters and predicts a drug's potential for clinical resolution of human disease sequelae. In addition, the rabbit eye is the definitive clinical model for examining HSV replication and its associated disease manifestations. Drug treatment parameters defined herein will be re-evaluated in the rabbit herpetic eye disease model scoring clinical parameters daily as defined in the accompanying table (FIG. 11) and the protocol outlined in FIG. 12.

Results, Analysis, and Alternative Approaches

The mouse and rabbit ocular models of HSV-mediated chronic eye disease are drug evaluation models that we have experience running for industry and NIH contract services. The clinical scoring and disease parameters have been validated and have been used for preclinical approval in FDA-IND and NDA filings for other drugs. From initial mouse data, without wishing to be bound by theory, the activities of 5-HT agonists will protect against virus and inflammation-associated eye disease.

### REFERENCES CITED IN THIS EXAMPLE

1. Risau W. Mechanisms of angiogenesis. Nature. 1997 Apr. 17; 386(6626):671-4. Review.
2. Greaves N S, Ashcroft K J, Baguneid M, Bayat A. Current understanding of molecular and cellular
mechanisms in fibroplasia and angiogenesis during acute wound healing. J Dermatol Sci. 2013 December; 72(3):206-17. doi: 10.1016/j.jdermsci.2013.07.008. Epub 2013 Jul. 30. Review.
3. Carmeliet P. Angiogenesis in health and disease. Nat Med. 2003 June; 9(6):653-60. Review
4. Folkman J. Angiogenesis in cancer, vascular, rheumatoid and other disease. Nat Med. 1995 January; 1(1):27-31. Review. 5. Chung A S, Ferrara N. Developmental and pathological angiogenesis. Annu Rev Cell Dev Biol. 2011; 27:563-84. doi: 10.1146/annurev-cellbio-092910-154002. Epub 2011 Jul. 13. Review.
10. Bradley J, Ju M, Robinson G S. Combination therapy for the treatment of ocular neovascularization. Angiogenesis. 2007; 10(2):141-8. Epub 2007 Mar. 13. Review.
11. Chen J, Smith L E. Retinopathy of prematurity. Angiogenesis. 2007; 10(2):133-40. Epub 2007 Feb. 27. Review.
12. Li W, Man X Y, Chen J Q, Zhou J, Cai S Q, Zheng M. Targeting VEGF/VEGFR in the treatment of psoriasis. Discov Med. 2014 September; 18(98):97-104. Review.
13. Di Rosso M E, Palumbo M L, Genaro A M. Immunomodulatory effects of fluoxetine: A new potential pharmacological action for a classic antidepressant drug?Pharmacol Res. 2015 Nov. 28. pii: S1043-6618(15)00279-0. doi: 10.1016/j.phrs.2015.11.021. [Epub ahead of print] Review.
14. Worthington J J. The intestinal immunoendocrine axis: novel cross-talk between enteroendocrine cells and the immune system during infection and inflammatory disease. Biochem Soc Trans. 2015 August; 43(4):727-33. doi: 10.1042/BST20150090. Epub 2015 Aug. 3. Review.
15. Nau F Jr, Yu B, Martin D, Nichols C D. Serotonin 5-HT2A receptor activation blocks TNF-α mediated inflammation in vivo. PLoS One. 2013 Oct. 2; 8(10):e75426. doi: 10.1371/journal.pone.0075426. eCollection 2013.
16. Yu B, Becnel J, Zerfaoui M, Rohatgi R, Boulares A H, Nichols C D. Serotonin 5-hydroxytryptamine(2A) receptor activation suppresses tumor necrosis factor-alpha-induced inflammation with extraordinary potency. J Pharmacol Exp Ther. 2008 November; 327(2):316-23. doi: 10.1124/jpet.108.143461. Epub 2008 Aug. 15.
18. Whitcher J P, Srinivasan M, Upadhyay M P. Corneal blindness: a global perspective Bulletin of the World Health Organization, 2001, 79: 214-221.
19. Facts about the cornea and corneal disease. National Eye Institute website, March 2010. Available at http://www.nei.nih.gov/health/cornealdisease/
20. Liesegang T J. Herpes simplex virus epidemiology and ocular importance. Cornea. 2001 January; 20(1): 1-13. Review.
21. Rowe A M, St Leger A J, Jeon S, Dhaliwal D K, Knickelbein J E, Hendricks R L. Herpes keratitis. Prog Retin Eye Res. 2013 January; 32:88-101.
22. Pavan-Langston, D., Foster, C. S., Trifluorothymidine and idoxuridine therapy of ocular herpes. Am. J. Ophthalmol. 1977. 84, 818-825.
23. Imperia P S, Lazarus H M, Dunkel E C, Pavan-Langston D, Geary P A, Lass J H. An in vitro study of ophthalmic antiviral agent toxicity on rabbit corneal epithelium. Antiviral Res. 1988 July; 9(4):263-72.
24. Choong K, Walker N J, Apel A J, Whitby M. Aciclovir-resistant herpes keratitis. Clin Experiment Ophthalmol. 2010 April; 38(3):309-13.
25. Piret J, Boivin G. Resistance of herpes simplex viruses to nucleoside analogues: mechanisms, prevalence, and management. Antimicrob Agents Chemother. 2011 February; 55(2):459-72.
26. Kaufman H E. Treatment of viral diseases of the cornea and external eye. Prog Retin Eye Res. 2000 January; 19(1):69-85.
27. Aquavella J V, Gasset A R, Dohlman C H. Corticosteroids in corneal wound healing. Am J Ophthalmol 1964; 58: 621-6.
28. Mitsui Y, Hanabusa J. Corneal infections after cortisone therapy. Br J Ophthalmol 1955; 39: 244-50.
29. Sharif N A. Serotonin-2 receptor agonists as novel ocular hypotensive agents and their cellular and molecular mechanisms of action. Curr Drug Targets. 2010 August; 11(8):978-93. Review.
30. Gudjonsson J E, Elder J. Psoriasis. In: Wolff K, Goldsmith L A, Katz S I, Gilchrest B A, Paller A S, Leffell D J, editors. Fitzpatrick's Dermatology in General Medicine. 7. New York: McGraw-Hill; 2008. p. 169.
31. Zaba L C, Cardinale I, Gilleaudeau P, Sullivan-Whalen M, Suxrez-Farifias M, Fuentes-Duculan J, Novitskaya I, Khatcherian A, Bluth M J, Lowes M A, Krueger J G. Amelioration of epidermal hyperplasia by TNF inhibition is associated with reduced Th17 responses. J Exp Med. 2007; 204:3183-94. doi: 10.1084/jem.20071094.

### Example 4

The 5HT agonist, DOI, inhibits HSV-1 neuronal reactivation from latency within trigeminal ganglia. 14 trigeminal ganglia from 7 ocularly infected mice that contained latent HSV-1 genomes within its neurons for greater than 60 days were removed, randomly divided into 2 groups of 7 ganglia, and were subsequently explanted and eviscerated in media that contained either 500 nM of DOI or an equivalent buffer control without drug. HSV-1 reactivation from latent neurons was induced using hyperthermic shock (42C) for 1 hour. Each day for 10 days post explant and induction of reactivation, ⅕ volume of media volume was removed and assessed for the presence of infectious HSV-1, indicating reactivation of virus from latency. This volume was replaced with media that contained either 500 nM of DOI drug or an equivalent of mock carrier buffer.

The 5HT agonist, DOI, maintains latency of HSV-1 within reactivation induced neurons as observed by the number and percentage of neurons positive for the presence of any infectious HSV-1. In addition, there was a significant delay in reactivation (2 fold greater) of HSV-1 from TGs that showed slight positivity for eventual presence of infectious virus.

| | Days post Explant | | | | | | | | | |
|---|---|---|---|---|---|---|---|---|---|---|
| | 1 | 2 | 3 | 4 | 5 | 6 | 7 | 8 | 9 | 10 |
| DOI 500 nM | 0/7 (0%) | 0/7 (0%) | 0/7 (0%) | 0/7 (0%) | 0/7 (0%) | 0/7 (0%) | 0/7 (0%) | 1/7 (14.3%) | 1/7 (14.3%) | 2/7 (28.6%) |
| Mock | 0/7 (0%) | 0/7 (0%) | 0/7 (0%) | 0/7 (0%) | 2/7 (28.6%) | 4/7 (57.1%) | 4/7 (57.1%) | 5/7 (71.4%) | 5/7 (71.4%) | 5/7 (71.4%) |

In addition, the 5HT agonist, DOI significantly inhibited the degree of reactivation and amount of infectious virus shed from latent neurons. Analysis of average total reactivated infectious virus (PFU/ml/TG) or total reactivated infectious virus per positive TG (PFU/ml/positive TG) both indicate that DOI suppressed HSV reactivation, active replication, and shedding of infectious virus from latent neurons relative to mock treated neurons.

### Example 5

Introduction

Globally, infection- and inflammation-associated eye diseases are the leading causes of corneal blindness and visual morbidity, with over 500 million individuals affected¹. As used herein in this example, “inflammation” and “inflammatory”, can be used interchangeably with “hypersensitivity.” The model ocular viral pathogen in these studies, Herpes Simplex virus type I (HSV-1), is present in 70-90% of the population². Indeed, herpetic keratitis is the leading cause of infectious corneal blindness in developed countries^{2,3}. The National Eye Institute estimates that 450,000 Americans have experienced some form of ocular herpetic disease, with 50,000 new and recurrent cases diagnosed². Current antiviral drugs fail to inhibit pathogen-induced inflammatory responses^{3,4}. As such, approximately 25% of cases present with serious inflammation-associated stromal keratitis. Individuals that have experienced ocular herpes have a 50% chance of recurrence². Each repeated episode triggers a chronic inflammatory response that can result in vascularization and subsequent vision-threatening scarring of the cornea, eventually requiring corneal transplantation for resolution³⁵.

Immuno-suppressive drugs, such as dexamethasone, can control deleterious inflammation⁶; however, they also license uncontrolled pathogen replication and are associated with loss of an intact corneal epithelial barrier, increased ocular pressure and eventual deterioration of vision^{7,8}. A role in modulating inflammatory processes was discovered for the serotonin receptor family, also known as the 5-hydroxytryptamine receptors (5HT)^{9,10}. Once thought only to be involved in modulating release of neurotransmitters in the central and peripheral nervous system, these GPCRs are finding new and unexpected life as modulators of broader biological functions, including in cardiovascular biology and as immune regulators. Recent studies have shown that modulation of 5HT_{2A} receptor activity within the eye can decrease ophthalmic pressure¹¹.

Aspects of the invention are directed towards DOI, a 5HT2R agonist, that holds promise for the treatment of herpetic keratitis. In studies, (R)-DOI has demonstrated anti-inflammatory and anti-vascularization properties in mouse models of primary and chronic herpetic keratitis. In addition, in ex vivo neuronal models, (R)-DOI inhibited HSV-1 reactivation from latency, a main contributor to development of recurrent herpetic stromal keratitis. Without wishing to be bound by theory, experiments described herein will show that (R)-DOI ameliorates inflammation and vascularization associated with herpetic keratitis, optimize formulation and dosing parameters for effective (R)-DOI ophthalmic delivery, and establish ophthalmic (R)-DOI's CNS safety profile, which will be accomplished using the following strategy:

(I) Determine Ophthalmic Formulation Tolerability and Pharmacokinetic Parameters of (R)-DOI

A series of experiments designed to determine dosing parameter will be performed, including ocular tolerability of (R)-DOI in topical formulation, drug distribution and pharmacokinetic evaluation of (R)-DOI in the rabbit eye. Importantly, this information will be used to investigate therapeutic efficacy of (R)-DOI relative to the current standard of care antiviral (trifluorothymidine; TFT) and anti-inflammatory (dexamethasone) treatments.

(II) Further Validate that (R)-DOI Controls Clinical Manifestations Associated with Both Acute and Chronic Herpetic Keratitis Using Three Complementary Animal Models

Mouse model data indicates therapeutic efficacy at preventing formation of blinding herpetic stromal keratitis. Mouse studies will be performed, specifically examining therapeutic efficacy in models that are directly relevant to human clinical herpes-associated chronic and recurrent disease. In addition, these studies will be complemented by examining therapeutic efficacy in an acute herpetic keratitis rabbit eye model—a model that has demonstrated predictive ability in development of topical ocular therapeutics for viral- and inflammation-mediated diseases.

Without wishing to be bound by theory, these studies will establish efficacy, optimal ocular delivery, and dosing parameters for a new treatment approach to herpetic keratitis. If targeting 5HT₂ receptors in herpetic keratitis controls inflammation without all the negative side effects of current standard treatments (e.g., dexamethasone), and additionally reduces vascularization and ocular pressure, such approach will address two of the disease pillars that are currently not easily treatable in numerous ocular conditions. In this regard, these efforts will validate the use of embodiments of the invention for other inflammation-associated ocular diseases.

Significance:

Herpetic keratitis in the cornea affects 1.5 million people worldwide and causes 40,000 new cases of partial or full blindness each year¹². Blindness occurs from corneal structural damage caused by prolonged inflammation from latent and recurrent Herpes Simplex Virus (HSV) infection^{13,14}. HSV infections of the eye are the leading cause of infectious corneal blindness in developed countries¹⁵ and approximately 500,000 people in the US are currently infected with ocular HSV¹⁶. The costs of treatment for this disease are in the tens of millions spent annually in the US alone¹⁷.

Current treatments range from topical eyedrops to gene therapy; however, anti-viral eyedrops can cause corneal epithelial toxicity¹⁸, and while anti-inflammatory steroid eyedrops are initially effective, they increase susceptibility to fungal infections⁸. Gene therapy to target the HSV genome, inflammatory mediators, the neovascularization process, or the viral receptors on host cells are limited by achieving appropriate vector expression level, bioavailability, and serotype specificity¹⁹. None of these treatments provide a cure, but rather decrease symptom duration and promote virus latency leaving the patient at risk to develop corneal scarring and blindness with each subsequent episode. Corneal scarring that leads to blindness is an indication for corneal transplantation, but transplantation in patients with HSK is complicated by an increased risk of graft rejection²⁰. Thus, the significant side effects or lack of specificity and penetrance for current treatments result in persistence of herpetic keratitis and represent an unmet need. One approach for an effective treatment is to eliminate the prolonged inflammatory episodes associated with HSV infection to reduce corneal damage and blindness. Embodiments as described herein comprise a new drug to treat herpetic keratitis-associated eye inflammation that will target the 5-hydroxytryptamine receptor 2A (aka serotonin/5HT_{2A} receptor). Serotonin or 5-hydroxytryptamine (5HT) is a small monoamine molecule primarily known for its role as a neurotransmitter. Within the brain, 5HT modulates a variety of behaviors including cognition, mood, aggression, mating, feeding, and sleep²¹. These behaviors are mediated through interactions at seven different receptor families (5HT1-7) comprised of fourteen distinct subtypes; of all the serotonin receptors, the 5HT_{2A} receptor, which is known to primarily couple to the Gαq effector pathway²², has been the one most closely linked to complex behaviors. Agonists of the serotonin receptor pathway, such as (R)-DOI, can be delivered systemically or topically (e.g., through a topical ocular drop) to prevent and resolve pathogen-elected host-mediated disease processes. Through this approach, embodiments such as (R)-DOI will promote an anti-inflammatory state to reduce repetitive scarring and neovascularization from recurrent HSV episodes, leading to reduced blindness.

Individuals with ocular herpes have a 50% chance of recurrence²³. Each episode triggers a chronic inflammatory disease process that results in inflammation, neovascularization and subsequent vision-threatening corneal scarring. Embodiments herein comprise a new class of non-steroidal anti-inflammatory drugs that target the 5HT receptors, such as small molecule agonist drug (R)-DOI. Current steroidal anti-inflammatory drugs, such as dexamethasone, control deleterious inflammation, but license uncontrolled pathogen replication. Furthermore, they reduce corneal epithelial barrier integrity, increase intraocular pressure, and may not prevent deterioration of vision²⁴. While modulation of 5HT receptor activity within the eye has been shown to decrease ophthalmic pressure²⁵, 5HT receptor targeting as a method to reduce HSV-associated inflammation has never been explored.

(R)-DOI injected intraperitoneally at doses up to 30-fold below the CNS behaviorally-active threshold dose in mice potently inhibits TNFα-induced inflammatory gene and protein expression. Markers inhibited include intercellular adhesion molecule 1 (Icam1), vascular cell adhesion molecule 1(Vcam1), interleukin 6 (116), interleukin 1 beta (IIIb), C—C motif chemokine ligand 2 (Ccl2/Mcp1), and C-X3-C motif chemokine ligand 1 (Cx3c11) in the aorta and intestine, and VCAM1 protein in the small intestine¹¹. Many of these same inflammatory molecules are expressed in herpetic keratitis infected corneas and participate in development of HSK^{26,27}. Without wishing to be bound by theory, targeting ocular 5HT_{2A} receptors may reduce herpetic keratitis-associated inflammation and ultimately reduce blindness by way of 5HT/5HT_{2A} receptors-mediated regulation of eye homeostasis, function, and health^{20,28}.

Supporting Data:

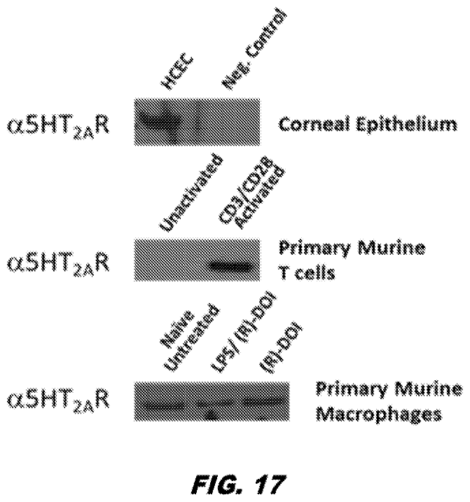

The corneal epithelia of human eyes, as well as activated immune cells that contribute to development of HSK, express 5HT_{2A} receptors. HSV infection of the eye can result in a recurrent inflammation-associated stromal keratitis that causes vascularization and vision-threatening scarring of the cornea^{3,5}. A variety of mechanisms regulates inflammation. One mechanism is through the G-protein coupled receptors (GPCRs) of the serotonin (5HT) receptor family. 5HT receptors not only modulate neurotransmitter release but are also recognized modulators of broad biological functions, including cardiovascular biology and immune regulation^{9,10}. Relevant to this is the 5HT_{2A} receptor. 5HT_{2A} receptor activation with the agonist (R)-DOI suppresses inflammation by inhibiting TNF-alpha activity^{29,30}, an important factor in the pathogenesis of recurrent herpetic keratitis²⁷. Further, 5HT_{2A} receptors are expressed in the eye and on activated immune cells that contribute to the development of HSK (FIG. 17). Without wishing to be bound by theory, this data indicates ocular 5HT-receptor modulation with (R)-DOI suppresses the development of HSK through modulation of TNF-associated inflammation.

Topical (R)-DOI reduces the development of acute and chronic herpetic keratitis-associated disease in a murine model. Following infection of the eye, HSV is associated with acute keratoconjunctivitis and dendritic or geographic ulceration of the cornea. The initial active viral replication is resolved approximately 8-10 days post-infection as the host response limits lytic replication of HSV and the virus goes latent within innervating neurons. Throughout a patient's lifetime, reactivation of latent HSV results in episodes of recurrent disease that induce inflammatory responses that result in vision-threatening immunopathic disease of the stroma. An ophthalmic formulation of (R)-DOI was developed and tested for its ability to reduce HSV-induced inflammation-associated ocular disease in a murine model of HSV-1 (RE) chronic herpetic keratitis. For example, R-DOI can be an active component in an ophthalmic balanced salt solution (BSS) that further comprises different amounts (i.e., percentages) and different viscosities of carrier compounds so as to maintain the visual field following drug delivery. For example, the solution can be formulated so that it has a refractive index similar to that of natural tears (1.33698). In an embodiment, the carrier compound can be carboxymethylcellulose (CMC). In an embodiment, the carrier compound is a carbonyl or carboxy polymer (i.e., carbopol) used as gelling agents to produce a composition of varying viscosities.

BALB/c eyes were scarified and infected with HSV-1 (RE), an HSV-1 strain that causes herpetic keratitis in 100% of ocular infections³¹. Starting 24 hours post-infection, topical drops were applied in a masked fashion 4× per day for 8 days. Individual clinical parameters were monitored and scored in a masked fashion for 15 days. Initially, during the acute herpetic keratitis stage, both the antiviral 1% TFT and (R)-DOI (500 M) exhibited reduced signs of disease, including reduced stromal opacity and corneal neovascularization.

TABLE 4
Day 15 days post-infection.

| | # Eyes | | |
|---|---|---|---|
| | showing | | |
| Treatment | signs of | | |
| group | disease | # Deceased | Clinically clear eyes |
| BSS drops | 7/8 | 2/6 | 0/8 |
| 1% TFT | 7/10 | 0/5 | 0/10 |
| (R)-DOI | 3/10* | 0/5 | 6/10 |

*The three eyes without clinically resolved disease still exhibited reduced clinical scores associated with ocular pathology.

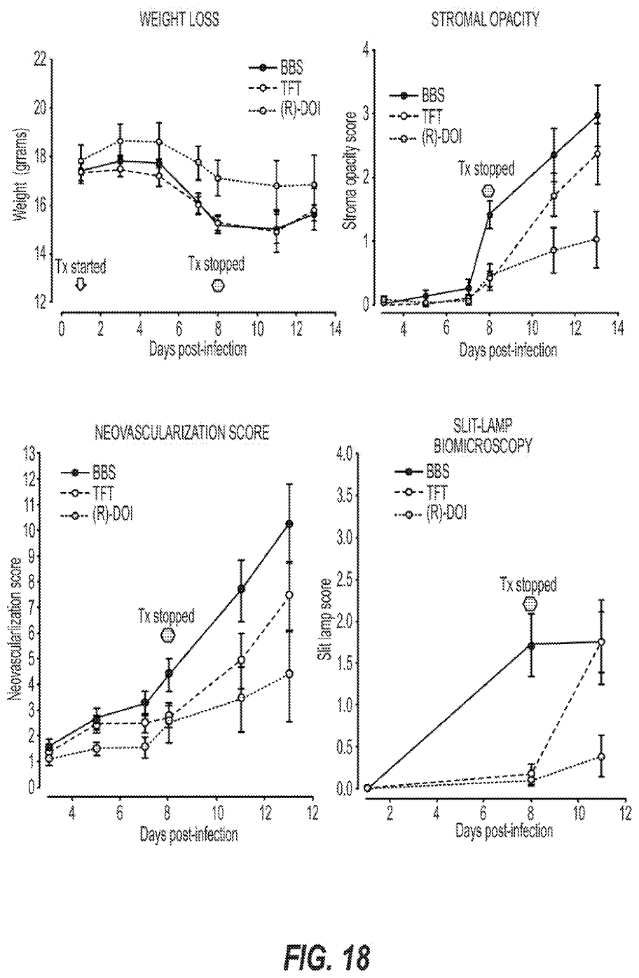

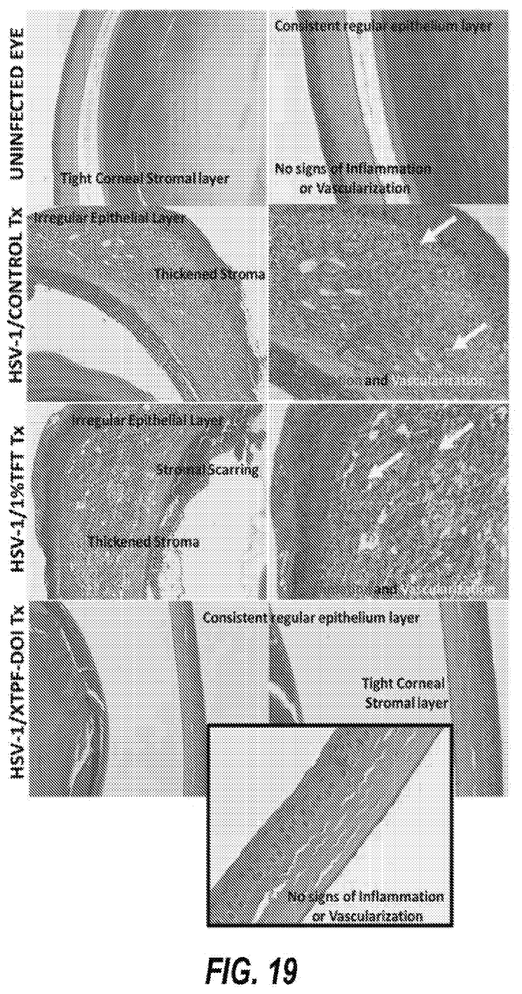

However, after day 8, as the chronic immuno-pathology processes progressed, only eyes treated with (R)-DOI showed significantly reduced disease relative to control BSS treatment (FIG. 18; Table 4), indicating that (R)-DOI effectively suppresses both virus- and inflammation-mediated disease processes. At day 15 post infection, a stage representative of chronic immune-associated disease and in the absence of virus, animals were euthanized, and the eyes were removed for histology. Random representative eyes were prepared by taking sections through the central cornea and processed by H&E histology for visualization (FIG. 19). Both BSS control and TFT treated HSV-1 infected eyes exhibited immune cell infiltration, thickening of the stroma, neovascularization, and disruption of the corneal epithelial layers. By contrast, (R)-DOI treated eyes epithelial and stromal layers were most similar to uninfected eyes. Taken together, these preliminary data strongly support the rationale for this proposal and call for further examination of (R)-DOI's therapeutic activity in additional acute, chronic, and recurrent herpetic keratitis ocular disease models that have proven human clinical translational value.

In contrast to current ocular anti-inflammatories, (R)-DOI suppresses neuronal reactivation of latent HSV-1, as well as suppresses its lytic replication. In order to preserve vision, the eye limits local immune and inflammatory responses. During trauma, infection, and in several ocular diseases, the normally immunoprivilaged nature of the eye can be disrupted resulting in sight-threatening chronic inflammation.

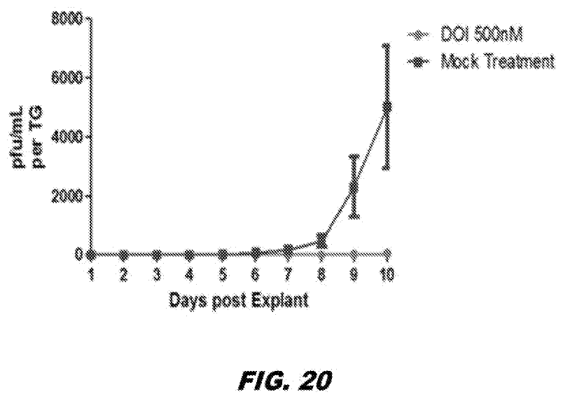

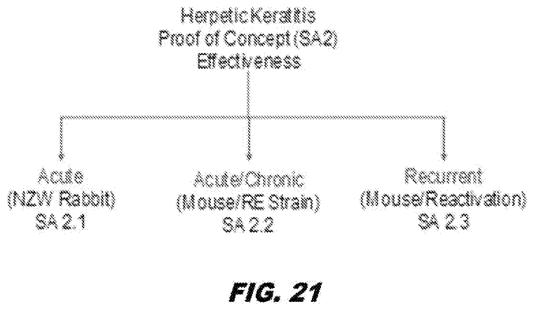

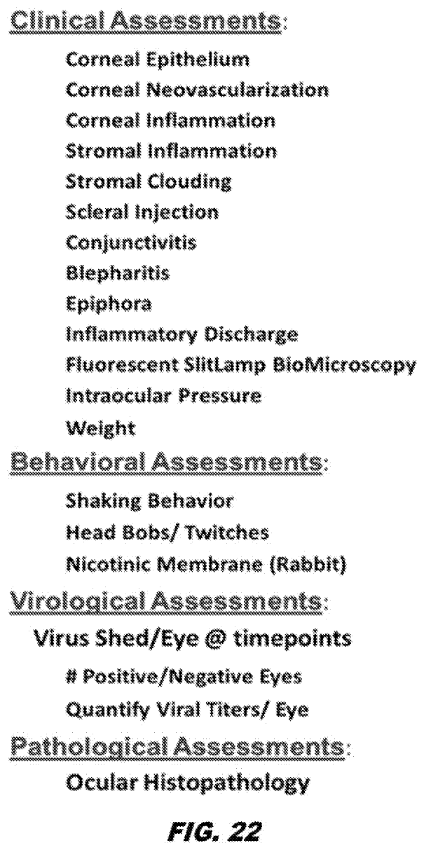

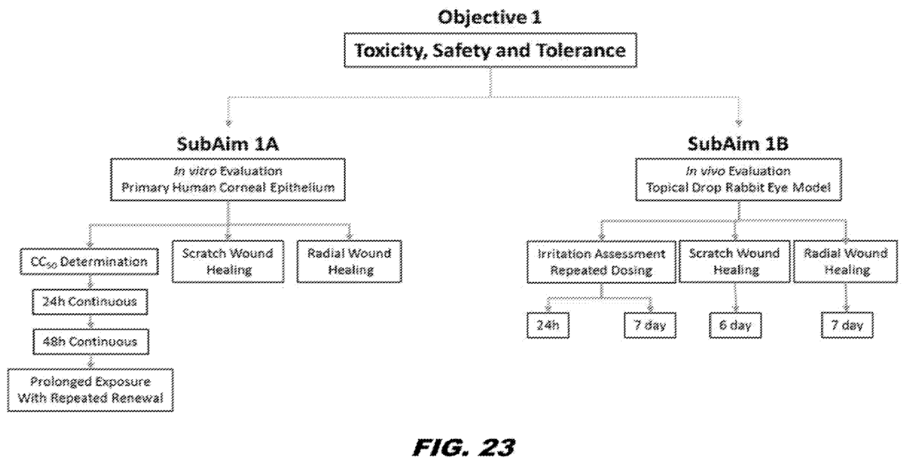

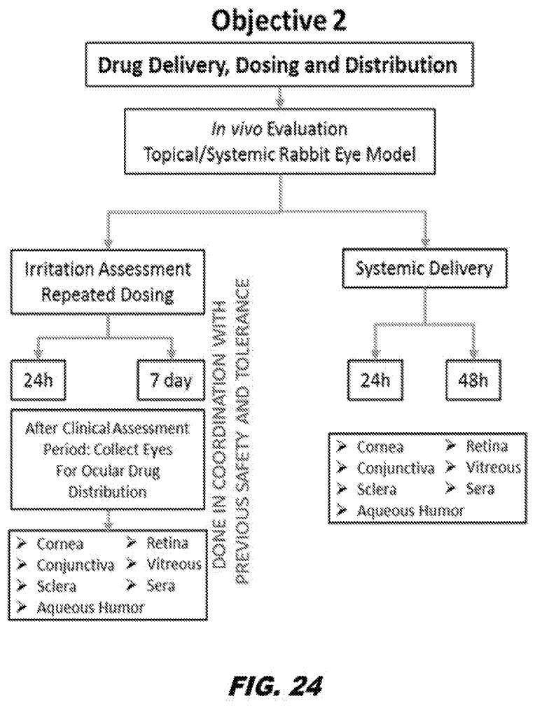

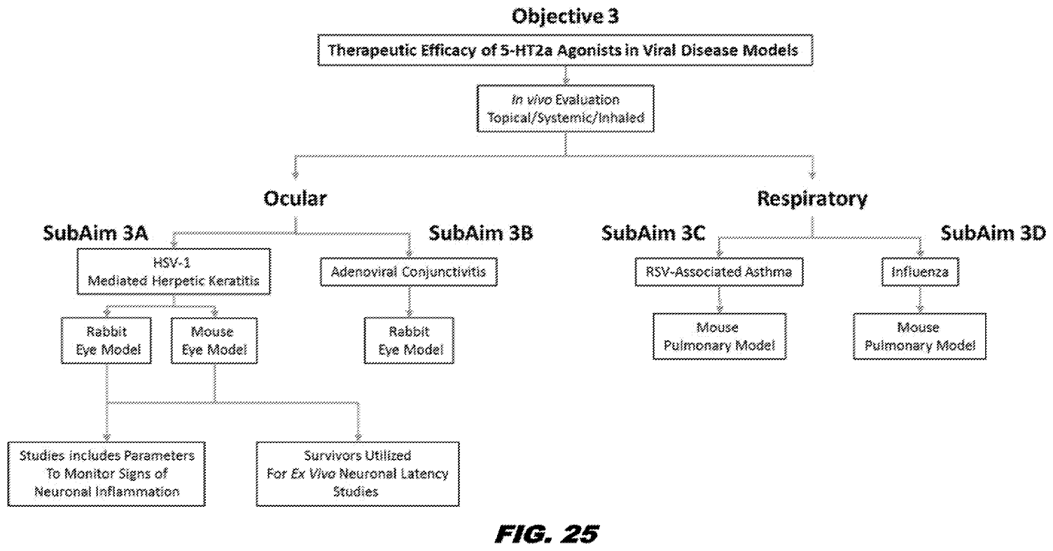

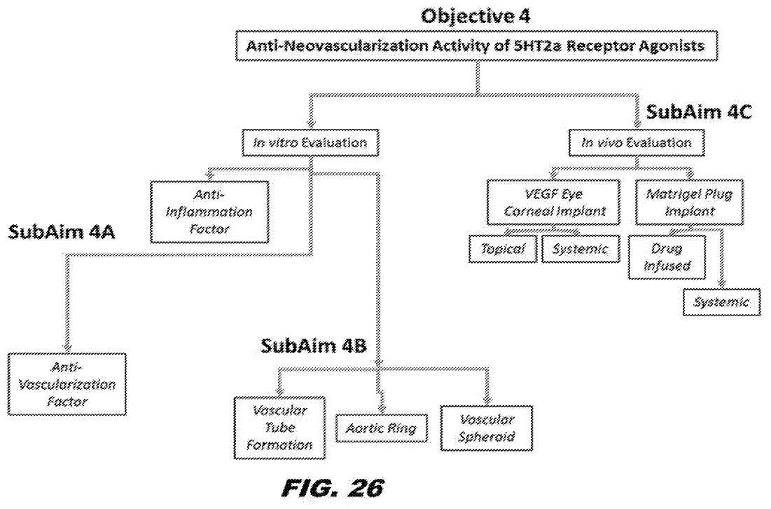

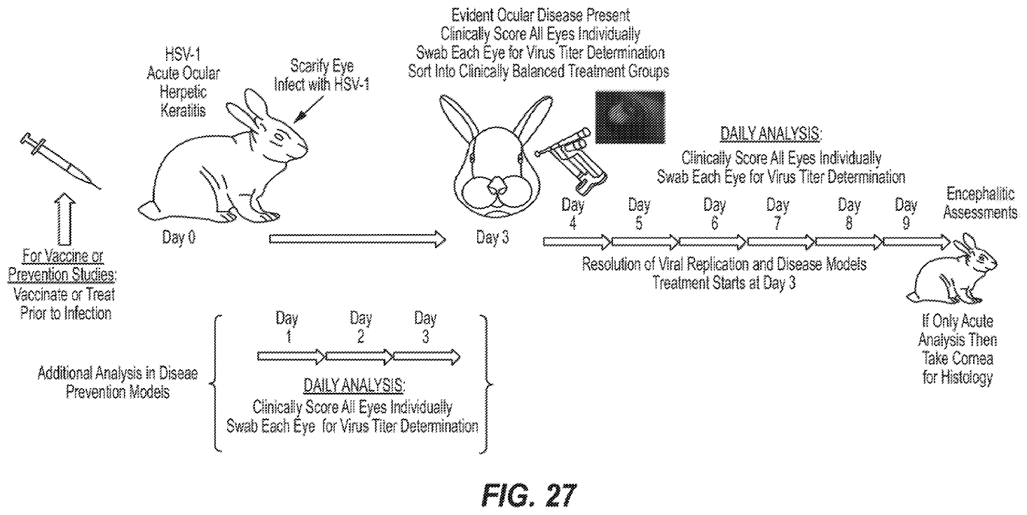

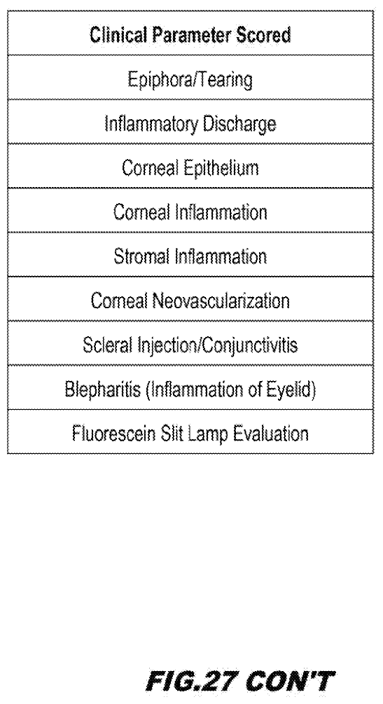

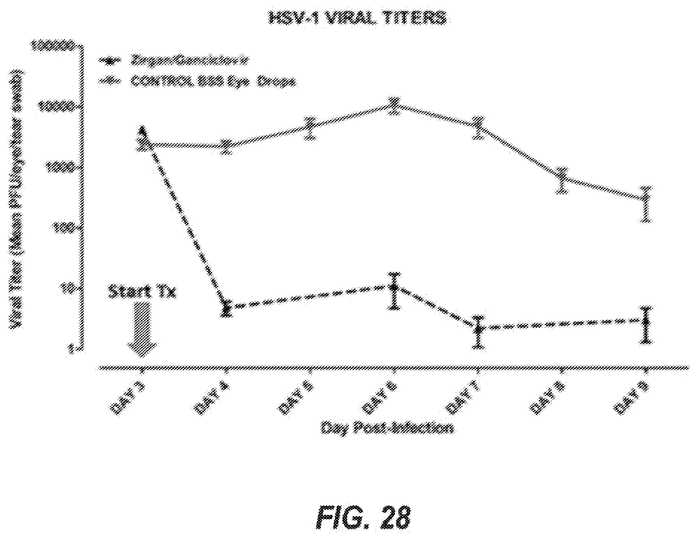

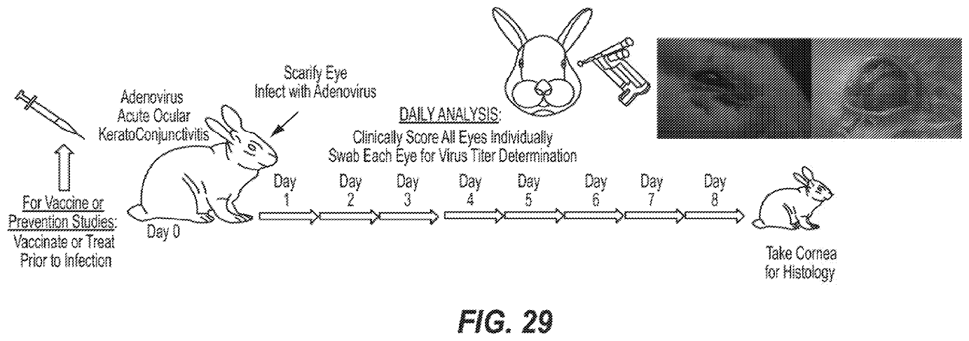

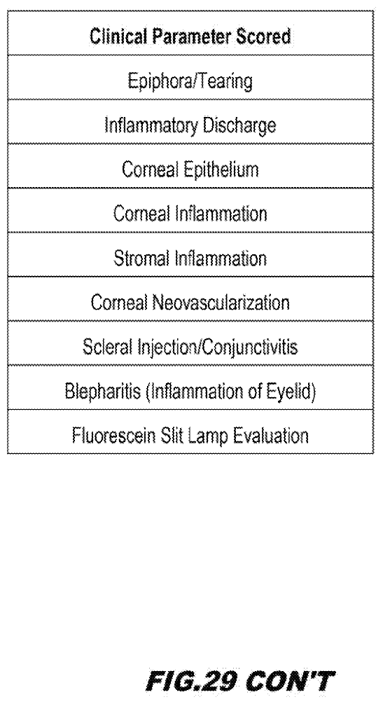

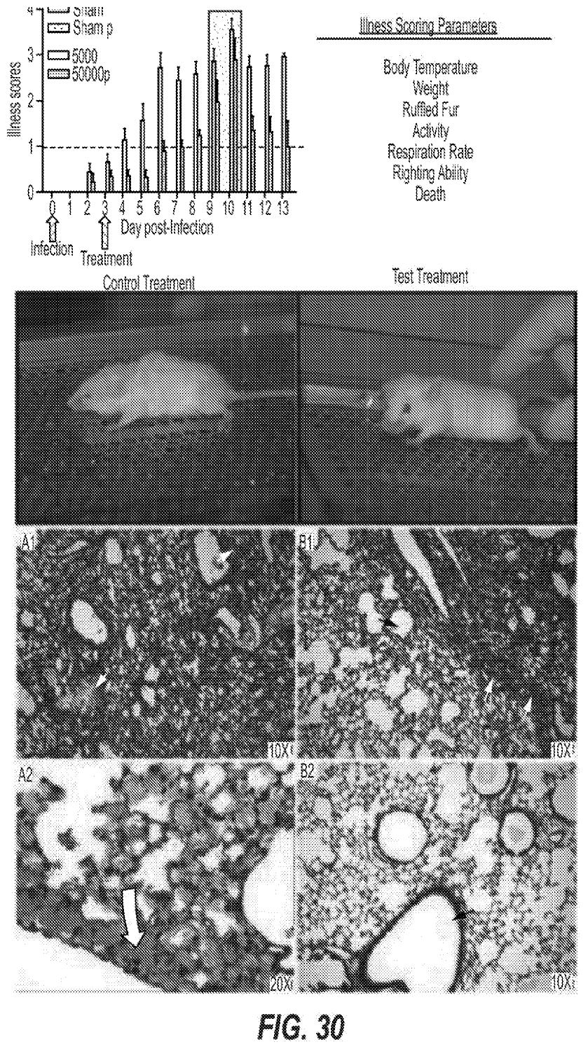

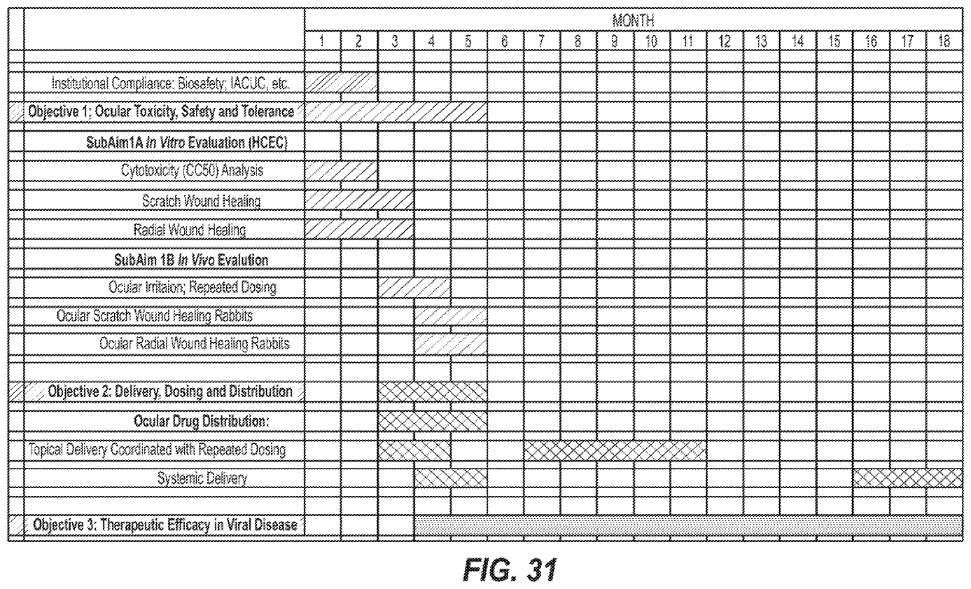

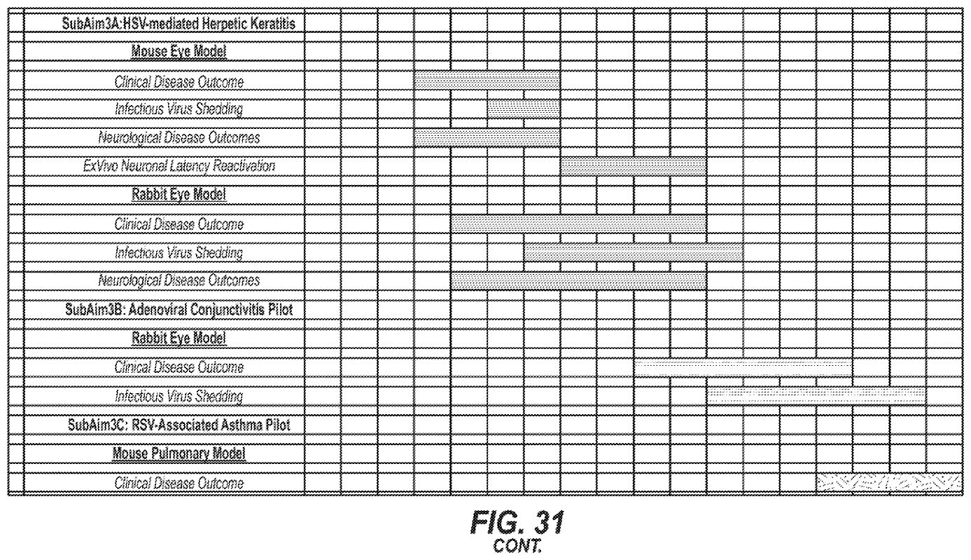

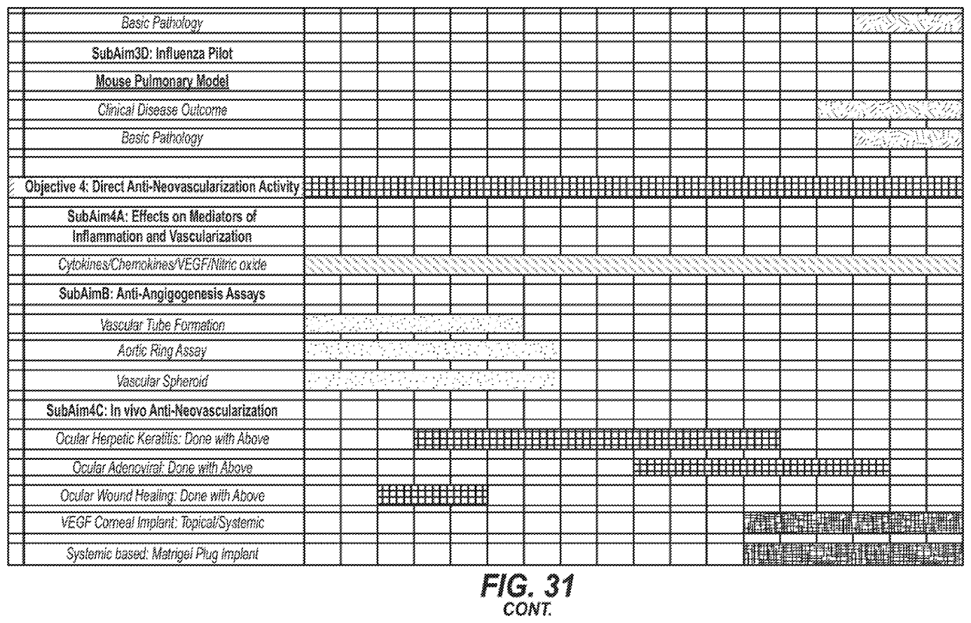

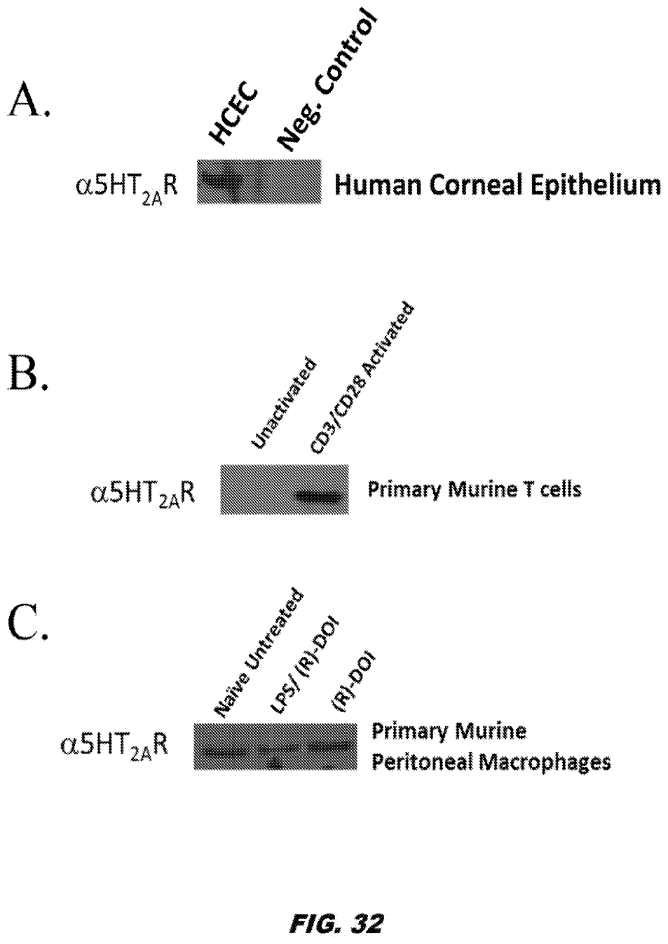

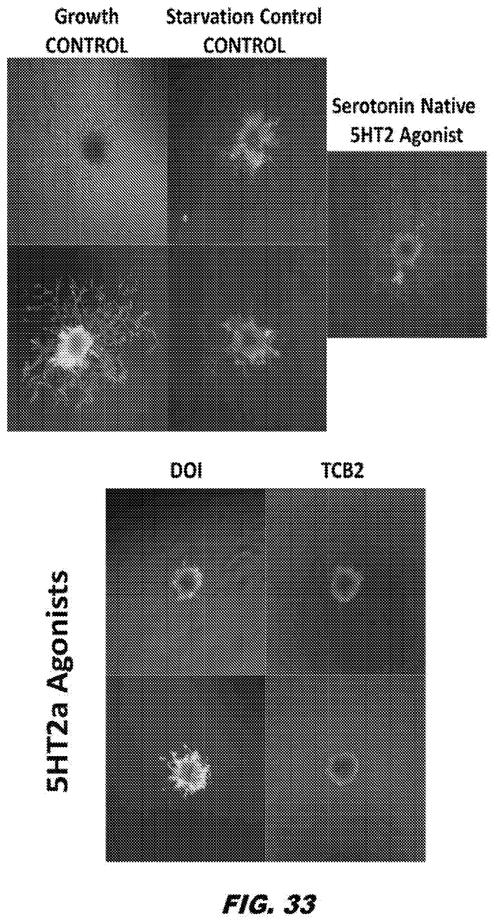

Although current ocular anti-inflammatories effectively suppress vision-threatening inflammation, they increase the risk of infection and license uncontrolled pathogen replication—a severe public health issue for pathogens such as Adenoviruses and Herpesviruses. In addition, post-surgical use of corticosteroids may trigger or worsen recurrent HSV keratitis by causing reactivation of the virus from latent neurons. Therefore, anti-inflammatory activity therapeutic development that suppress pathogen replication and/or prevent HSV-reactivation will represent a vastly superiority option to current immunosuppressive treatment options. To ascertain if (R)-DOI suppresses HSV-reactivation from latent neurons, 14 trigeminal ganglia (TG) from 7 ocularly infected mice that contained latent HSV-1 genomes within its neurons for greater than 60 days were removed, randomly divided into 2 groups of 7 ganglia, and were subsequently explanted and eviscerated in media that contained either 500 nM of DOI or an equivalent buffer control without drug. HSV-1 reactivation from latent neurons was induced using hyperthermic shock and each day for 10 days post-reactivation the presence of infectious HSV-1 was assessed (FIG. 20). (R)-DOI significantly inhibited the number of reactivating TGs and the amount of infectious virus shed from neurons. Analysis of average total reactivated infectious virus (PFU/ml/TG) indicated that (R)-DOI suppressed HSV reactivation, active replication, and viral shedding from latent neurons relative to buffer control treated neurons.

Determine Ophthalmic Formulation Tolerability and Dosing Parameters of (R)-DOI.

The establishment of a drug's toxicity, safety and tolerance profiles is a compulsory prerequisite to all subsequent efficacy trials. These profiles dictate a drug's practical concentrations, and properties they impart to carrier formulations that may alter tolerability (i.e. pH). In the exposed epithelium of the eye, which has regenerative and wound healing capacity that are critical for proper eye function, a drug formulation must not: 1) exhibit cellular toxicity to the corneal epithelium; 2) diminish cellular metabolic activity; 3) alter ocular physiological pH, which can burn the cornea; 4) inhibit replicative capacity of stem-like cells from the corneal limbus; or 5) impair epithelial migration/wound healing.

The rabbit remains the species of choice for the evaluation of ophthalmic compounds providing a relatively reliable model for the evaluation of ocular pharmacokinetics. Topical administration is the route of choice for the treatment of anterior segment diseases, most often with a local therapeutic effect. This route is non-invasive, painless and fast acting. In addition, the lower dosing requirements limit a drug's systemic effects³². Topical bioavailability is, however, often limited due to the precorneal loss increasing drug clearance and the corneal barrier limiting the distribution of drug. The absorption, drug distribution, and localized concentrations of (R)-DOI over time in conjunctiva, aqueous humor (AH), and cornea following ocular topical delivery on the rabbit eye will guide determination of clinical doses and posology in therapeutic paradigms of keratitis and across species. The study of the ability of (R)-DOI to penetrate and distribute across the different depth of ocular matrices posterior to cornea of the eye will also inform on additional potential therapeutic indications such as Uveitis. Further, describing pharmacokinetics in ocular target tissues is a challenge considering the eye's complex anatomy and its dynamic physiological protection. During drug development, animal and human pharmacokinetics can be assessed by sampling plasma at different time points. Determination of the levels of systemic exposure to (R)-DOI in the rabbit following ocular topical delivery will therefore inform future development studies where systemic exposure is of scope, and where plasma pharmacokinetics but not biopsies of the eye matrices for drug determination will be performed.

Overall Assessment Groups and Parameters:

For both the pharmacokinetic study and the tolerability study, conscious Dutch-Belted rabbits (see Vertebrate Animals document) will be administered test compound by topical application to the ocular surface of both eyes. 50 μL of formulated test article will be administered using a calibrated pipet. The lower eyelid will be pulled slightly off the ocular surface to act as a pocket and then released ˜15 seconds after administration. The vehicle for formulation will be 0.5% carboxymethylcellulose (CMC) in saline.

Examination of the Potential Ocular Tolerability in Male Dutch Belted Rabbits Following Topical Administration of (R)-DOI.

Experimental Approach:

The ocular tolerability of 3 (R)-DOI doses (low, mid- and high (100, 300, 1000 μM)) will be characterized, and vehicle alone following topical administration of the test formulation to both eyes 3 times daily (TID) for 4 days (N=2 rabbits/group, n=4 eyes/group, 50 μl per eye, for a total of 8 rabbits on study; Table 5). Draize scoring will be conducted pre-dose and on days 1, 3, & 5. Full ophthalmic exams will be performed pre-dose and prior to euthanasia on day 5. Eyes will be enucleated and fixed for histopathology.

TABLE 5
Experimental Groups in Strategy 1.

| | | Route and | | | | Terminal |
|---|---|---|---|---|---|---|
| Group | Test Article | Dose/Eye | Rabbits | Draize | Ophthalmic Exam | TimePoint |
| 1 | R-DOI | Topical, “Low” | N = 2/group | Predose | Slit lamp and indirect | Day 5 |
| | | both eyes TID for | | and Days 1, | ophthalmoscope of | |
| | | 4 days | | 3, & 5 | the front and back of | |
| 2 | R-DOI | Topical, “Mid” | | | the eye by a | |
| | | both eyes TID for | | | veterinary | |
| | | 4 days | | | ophthalmologist. | |
| 3 | R-DOI | Topical, “High” | | | Exams will be | |
| | | both eyes TID for | | | performed predose | |
| | | 4 days | | | and Day 5 using the | |
| 4 | Vehicle | Topical, Vehicle | | | McDonald Shadduck | |
| | | both eyes TID for | | | Scoring System | |
| | | 4 days | | | | |

Metrics:

This objective will establish foundational criteria of tolerability and toxicity to the eye of a range of doses of (R)-DOI and the functional parameters that can be employed within all subsequent in vitro and in vivo studies. Success of this objective relies on the determination of the (R)-DOI concentration range in a topical ophthalmic formulation that is compatible with therapeutic effects. Future studies including experimentation with a scratch wound healing model and a radial wound-healing model will be carried to determine tolerability and acceptable use of (R)-DOI to the damaged eye.

Alternative Approaches:

A potential limitation of ophthalmic drugs is low tolerability in ocular tissues. However, preliminary effective concentration doses are low (in the 100-500 μM range) and therefore we do not anticipate irritability or pH-changing properties of the drug at useful therapeutic doses for a limited period (e.g. 4 days). Significantly longer chronic administration can result in tolerability issues, especially if the drug accumulates in ocular tissues upon repetitive administrations. To address this, the determination of ocular pharmacokinetics and parameters such as area under the curve (AUC) levels and half-life will inform dosing regimen for repeated drug administration aimed at achieving near-steady-state drug levels in target ocular tissues (rate of drug elimination compensates the rate of drug administration).

Topical Ocular Pharmacokinetic Study in Male Dutch Belted Rabbits Using (R)-DOI as a Treatment for Herpetic Keratitis.

Experimental Approach:

The ocular exposure of (R)-DOI following a single ocular topical administration of the test formulation to both eyes will be characterized. Doses will be administered one time to both eyes of each rabbit. Animals will be euthanized immediately prior to the following time points: 0.25, 0.5, 1, 3, 6, & 24 h post-administration. Precise dissection and processing of ocular tissues conjunctiva, iris-ciliary body, vitreous humor, retina, choroid and cornea will be performed, and aqueous humor (anterior chamber), and plasma will be collected from each animal for determination of drug levels. Two animals (n=4 eyes) will be used at each time point for a total of 12 rabbits. Chromatography-tandem mass spectrometry (LC-MS/MS) method development and set-up for sample analysis of plasma and ocular matrices for (R)-DOI will employ n=144 ocular samples (24 eyes×6 matrices) and n=12 plasma samples³³.

Metrics:

This study will estimate first dose pharmacokinetic parameters (i.e. Tmax, Cmax, AUC0-t, AUC0-∞, T1/2, CL) of ophthalmic administration of (R)-DOI in ocular conjunctiva, iris-ciliary body, vitreous humor, retina, choroid, cornea, aqueous humor, and plasma in a model closely relevant to humans.

Alternative Approaches:

The determination of drug concentrations in different matrices is subject to the sensibility, linearity, quantifying and detection limits of the analytical methods employed during the study. LC-MS/MS analytical technology of drug quantification is considered one of the most appropriate approaches for that end³³. It offers analytical specificity superior to that of conventional high performance/pressure liquid chromatography (HPLC) for low molecular weight analytes and has higher throughput than gas chromatography-mass spectrometry (GC-MS). The preliminary estimate limit of detection (LOD), limit of quantitation (LOQ), and upper limit of linearity (ULOL) are in the range of 5-5-1000 ng/ml. In this study, (R)-DOI will be administrated at a single dose superior to the high therapeutic dose but inferior to the maximum tolerated ocular dose for acute single administration.

Further Validate that (R)-DOI Controls Clinical Manifestations Associated with Both Acute and Chronic Herpetic Keratitis Using Three Complementary Animal Models.

The development of herpetic keratitis is due both to viral and host-mediated processes, which result in chronic and recurrent disease manifestations that are not effectively controlled by current antiviral therapeutics. This strategy will validate (R)-DOI's ability to control disease manifestations associated with acute, chronic, and recurrent herpetic keratitis without the deleterious consequences associated with anti-inflammatories, such as uncontrolled viral replication and increased intraocular pressure.

TABLE 6
Treatment groups in Strategy 2.

| Assessment | Drug Treatment | Doses |
|---|---|---|
| Arm | Group | Administration |
| Treatment Control | Ophthalmic BSS | BSS: Topical |
| Antiviral | Trifluorothymidine | 1%: Topical |
| | (TFT) | |
| Anti-Inflammatory | Dexamethasone | 0.1%: Topical |
| Test High R-DOI | R-DOI in BSS | 500 μM: Topical |
| Test Low R-DOI | R-DOI in BSS | 8 μM: Topical |

Overall Assessment Groups and Parameters:

Each of the sub-strategies will follow a similar experimental design outline with 5 arms (summarized in Table 6) that will assess the effect of 2 doses of (R)-DOI relative to: 1) control BSS treatments; 2) the antiviral drug TFT; or 3) anti-inflammatory dexamethasone. All treatment groups will be masked by color coding. For each strategy, separate clinical, behavioral, and virological assessments will be scored daily by independent investigators masked to treatment. At the end of each protocol, eyes will be enucleated, and histopathology will be performed.

To Determine the Therapeutic Efficacy of (R)-DOI for Resolution of Acute HSV Keratitis in a Rabbit Eye Model.

The rabbit eye model of HSV-1 infection has been established as a gold-standard small-animal model assessment of a drug's ability to affect HSV-mediated acute ocular disease. The rabbit eye model of acute HSV-1 infection closely mimics the virological, as well as the inflammation- and neovascularization-associated clinical parameters of a human infection³⁴. Unlike other studies in the mouse eye, the rabbit eye is in many respects more morphologically similar to the human eye and viral replication and the acute herpetic keratitis disease course ensues like human disease. As such, it has been shown to robustly predict pharmaceutical efficacy of topical therapeutics. Ocular pharmacological parameters established in Strategy I can be correlated with disease outcomes and utilized to optimize future dosing and treatment regimens.

Experimental Approach:

New Zealand White rabbits (7 per treatment group; n=14 eyes) will have the corneas of both eyes scarified in a 4×4 cross-hatched pattern and immediately inoculated with 3×10⁵ PFU of HSV-1 suspended in 50 μl of ophthalmic BSS. To assess treatment effects on infection resolution and clinical disease, infection will proceed unabated for 3 days at which time animals will be clinically scored and accordingly sorted into clinically balanced groups prior to beginning treatment. This process normalizes inherent differences between animals and recapitulates the clinical scenario of a person reporting to the clinic with the onset of herpetic lesions. Topical drugs will be administered 4× daily. As depicted in Table 3, each morning scores for each clinical disease parameters will be assessed by slit lamp biomicroscopy. In addition, intraocular pressure will be determined each morning and just prior to last treatment using a Tonovet rebound tonometer. Infectious virus will be collected from the tears daily in order to assess drug effects on viral replication. To determine if drug treatments have any deleterious effects on behavior, the behavior will be monitored according to the parameters defined herein, briefly prior to each treatment and for a continuous 15 minutes following last daily treatment. At the end of the acute disease study, histological assessment of the eye will be performed to visualize what has been scored clinically and virologically.

To Determine the Therapeutic Efficacy of (R)-DOI for Prevention of Acute and Chronic HSV Keratitis in a Mouse Eye Model.

Although the acute rabbit eye model effectively assesses virological, clinical and pharmacological parameters of drug studies, the acute model does not efficiently permit assessment of chronic host-mediated factors that contribute to herpetic stromal keratitis. Infection of BalbC mice with HSV-1 (RE) strain results in nearly 100% of animals developing blinding herpetic stromal keratitis, with a large percentage developing disease despite effective suppression of viral replication by antivirals^{35,36}. Therefore, this model will be used to assess the effects of (R)-DOI on host-mediated chronic HSV-associated ocular disease development.

Experimental Approach: 9-week-old Balb/C mice (10 mice per group; n=20 eyes) will have the corneas of both eyes scarified in a 4×4 cross-hatched pattern and immediately inoculated with 5×10³ PFU of HSV-1 (RE) strain suspended in 5 □l of ophthalmic BSS. Animals will be randomly assigned to treatment groups as in Table 3 and drugs or specific controls will be administered 4× daily beginning at 3 hours post-infection. Daily clinical, behavioral and virological assessments beginning at 24 hours post-infection until day 10 will be performed. At approximately 8-9 days post-infection, viral titers are nearly undetectable in surviving animals and the host-mediated disease processes start. After the initial 10 days, clinical and virological assessments will continue every other morning until 20 days post infection upon which scoring the clinical disease parameters described herein by slit lamp biomicroscopy will be performed. Twenty days post-infection represents the time of peak chronic disease, thus animals will be sacrificed and eyes removed for histological examination. Infiltration of specific immune cells, vascularization, thickening of stroma and epithelium, and fibrotic scarring will be examined.

To Determine the Therapeutic Efficacy of (R)-DOI for Prevention of Recurrent Herpetic Keratitis in a HSV-Latency Reactivation Mouse Model^{37,38}.

Blinding herpetic stromal keratitis in humans occurs following years of HSV reactivation and recrudescent ocular disease. Although they have their usefulness in determining drug efficacy, the primary HSK models described herein do not recapitulate some aspects of HSK, which occur as a result of reactivating in the context of an immune host that developed an adaptive immune response against HSV. Therefore, this model will assess the effects of (R)-DOI following HSV reactivation and development of recurrent immune-mediated disease.

Experimental Approach:

To reduce mortality and prevent acute HSK during the primary infection, C57BL/6 mice (15 mice per group; n=30 eyes) will be IP administered normal human immunoglobulin prior to infection. The corneas of both eyes will be scarified in a 4×4 cross-hatched pattern and immediately inoculated with 1×10⁶ PFU of HSV-1 McKrae strain suspended in 5 μl of ophthalmic BSS. Six weeks following primary infection, eyes will be scored and animals with eyes that do not exhibit signs of ocular disease will be randomly sorted into assessment groups as described herein. HSV will be reactivated by exposure to UV-B light with a transilluminator, tear film collected for the presence of virus, and treatments will begin. Mice will be evaluated by a masked observer every 5 days for 25 days, at which time animals will be sacrificed and the eyes removed for histological examination.

Metrics and Alternative Approaches:

Data has indicated that (R)-DOI suppresses deleterious HSV-induced inflammation-associated disease in these models of herpetic keratitis. Without wishing to be bound by theory, (R)-DOI will be effective at suppressing disease sequelae associated with acute, chronic and recurrent HSK without increasing HSV replication or intraocular pressure. As such, it will exhibit superiority to current anti-inflammatory therapeutics. There are a few potential pitfalls with our approach and alternatives: 1) Despite our preliminary findings that (R)-DOI can significantly suppress HSV-neuronal reactivation and lytic replication in vitro, it is possible that it will not suppress HSV replication in vivo to an extent that will effectively prevent acute HSV-mediated disease processes. If suppression is not observed, as an alternative approach, (R)-DOI will be supplemented with an anti-herpetic to suppress viral replication, while still imparting its anti-inflammatory effects. In addition, because we have observed that (R)-DOI suppresses HSV reactivation from neurons in the reactivation recurrent disease model in SA2.2, we can administer (R)-DOI systemically at non-behavioral levels to suppress HSV-neuronal reactivation, if ocular administration does not impart this effect. 2) Despite the sub-behavioral levels of (R)-DOI being administered, the 5HT_{2A} agonist activity of (R)-DOI may induce behavioral effects with repeated long-term dosing, especially in the recurrent disease model of SA2.2. If this occurs, we will perform a dose-finding experiment to determine the lowest effective dose of (R)-DOI that excludes any behavioral effects.

### REFERENCES CITED IN THIS EXAMPLE

1 Whitcher, J. P., Srinivasan, M. & Upadhyay, M. P. Corneal blindness: a global perspective. Bulletin of the World Health Organization 79, 214-221 (2001).
2 National Eye Institute website, M. (Available at http://www.nei.nih.gov/health/cornealdisease/).
3 Rowe, A. M. et al. Herpes keratitis. Progress in retinal and eye research 32, 88-101, doi:10.1016/j.preteyeres.2012.08.002 (2013).
4 Imperia, P. S. et al. An in vitro study of ophthalmic antiviral agent toxicity on rabbit corneal epithelium. Antiviral research 9, 263-272 (1988).
5 Kaufman, H. E. Treatment of viral diseases of the cornea and external eye. Progress in retinal and eye research 19, 69-85 (2000).
6 Bian, F. et al. Differential Effects of Dexamethasone and Doxycycline on Inflammation and MMP Production in Murine Alkali-Burned Corneas Associated with Dry Eye. The ocular surface 14, 242-254, doi:10.1016/j.jtos.2015.11.006 (2016).
7 Aquavella, J. V., Gasset, A. R. & Dohlman, C. H. CORTICOSTEROIDS IN CORNEAL WOUND HEALING. American journal of ophthalmology 58, 621-626 (1964).
8 Mitsui, Y. & Hanabusa, J. Corneal infections after cortisone therapy. The British journal of ophthalmology 39, 244-250 (1955).
9 Di Rosso, M. E., Palumbo, M. L. & Genaro, A. M. Immunomodulatory effects of fluoxetine: A new potential pharmacological action for a classic antidepressant drug?Pharmacological research 109, 101-107, doi:10.1016/j.phrs.2015.11.021 (2016).
10 Worthington, J. J. The intestinal immunoendocrine axis: novel cross-talk between enteroendocrine cells and the immune system during infection and inflammatory disease. Biochemical Society transactions 43, 727-733, doi: 10.1042/bst20150090 (2015).
11 Sharif, N. A. Serotonin-2 receptor agonists as novel ocular hypotensive agents and their cellular and molecular mechanisms of action. Curr Drug Targets 11, 978-993 (2010).
12 Farooq, A. V. & Shukla, D. Herpes simplex epithelial and stromal keratitis: an epidemiologic update. Survey of ophthalmology 57, 448-462, doi:10.1016/j.survophthal.2012.01.005 (2012).
13 Tsatsos, M. et al. Herpes simplex virus keratitis: an update of the pathogenesis and current treatment with oral and topical antiviral agents. Clinical & experimental ophthalmology 44, 824-837, doi:10.1111/ceo.12785 (2016).
14 Tabbara, K. F. & Al Balushi, N. Topical ganciclovir in the treatment of acute herpetic keratitis. Clinical Ophthalmology (Auckland, N.Z.) 4, 905-912 (2010).
15 Darougar, S., Wishart, M. S. & Viswalingam, N. D. Epidemiological and clinical features of primary herpes simplex virus ocular infection. The British journal of ophthalmology 69, 2-6 (1985).
16 Lairson, D. R., Begley, C. E., Reynolds, T. F. & Wilhelmus, K. R. Prevention of herpes simplex virus eye disease: a cost-effectiveness analysis. Archives of ophthalmology (Chicago, Ill.: 1960) 121, 108-112 (2003).
17 Liesegang, T. J. Herpes simplex virus epidemiology and ocular importance. Cornea 20, 1-13 (2001).
18 Tabbara, K. F. & Al Balushi, N. Topical ganciclovir in the treatment of acute herpetic keratitis. Clinical ophthalmology (Auckland, N.Z.) 4, 905-912 (2010).
19 Elbadawy, H. M., Gailledrat, M., Desseaux, C., Ponzin, D. & Ferrari, S. Targeting herpetic keratitis by gene therapy. Journal of ophthalmology 2012, 594869, doi: 10.1155/2012/594869 (2012).
20 Yang, J. W., Xu, Y. C., Sun, L. & Tian, X. D. 5-hydroxytryptamine level and 5-HT2A receptor mRNA expression in the guinea pigs eyes with spectacle lens-induced myopia. International journal of ophthalmology 3, 299-303, doi: 10.3980/j.issn.2222-3959.2010.04.05 (2010).
21 Nichols, D. E. & Nichols, C. D. Serotonin receptors. Chemical reviews 108, 1614-1641, doi:10.1021/cr078224o (2008).
22 Roth, B. L. 5-HT(2A) SEROTONIN RECEPTOR BIOLOGY: Interacting proteins, kinases and paradoxical regulation. Neuropharmacology 61, 348-354, doi:10.1016/j.neuropharm.2011.01.012 (2011).
23 Al-Dujaili, L. J. et al. Ocular herpes simplex virus: how are latency, reactivation, recurrent disease and therapy interrelated? Future Microbiology 6, 877-907, doi:10.2217/fmb.11.73 (2011).
24 Hendricks, R. L., Barfknecht, C. F., Schoenwald, R. D., Epstein, R. J. & Sugar, J. The effect of flurbiprofen on herpes simplex virus type 1 stromal keratitis in mice. Investigative ophthalmology & visual science 31, 1503-1511 (1990).
25 Rocha-Sousa, A. et al. New Therapeutic Targets for Intraocular Pressure Lowering. ISRN Ophthalmology 2013, 261386, doi:10.1155/2013/261386 (2013).
26 Rolinski, J. & Hus, I. Immunological aspects of acute and recurrent herpes simplex keratitis. Journal of immunology research 2014, 513560, doi: 10.1155/2014/513560 (2014).
27 Keadle, T. L. et al. IL-1 and TNF-alpha are important factors in the pathogenesis of murine recurrent herpetic stromal keratitis. Investigative ophthalmology & visual science 41, 96-102 (2000).
28 Celada, P., Puig, M. V., Amargós-Bosch, M., Adell, A. & Artigas, F. The therapeutic role of 5-HT(1A) and 5-HT(2A) receptors in depression. Journal of Psychiatry and Neuroscience 29, 252-265 (2004).
29 Nau, F., Jr., Yu, B., Martin, D. & Nichols, C. D. Serotonin 5-HT2A receptor activation blocks TNF-alpha mediated inflammation in vivo. PloS one 8, e75426, doi:10.1371/journal.pone.0075426 (2013).
30 Yu, B. et al. Serotonin 5-hydroxytryptamine(2A) receptor activation suppresses tumor necrosis factor-alpha-induced inflammation with extraordinary potency. The Journal of pharmacology and experimental therapeutics 327, 316-323, doi: 10.1124/jpet.108.143461 (2008).
31 Deshpande, S. P. et al. Herpes simplex virus-induced keratitis: evaluation of the role of molecular mimicry in lesion pathogenesis. Journal of virology 75, 3077-3088, doi:10.1128/jvi.75.7.3077-3088.2001 (2001).
32 Patel, A., Cholkar, K., Agrahari, V. & Mitra, A. K. Ocular drug delivery systems: An overview. World journal of pharmacology 2, 47-64, doi:10.5497/wjp.v2.i2.47 (2013).
33 Schopf, L., Enlow, E., Popov, A., Bourassa, J. & Chen, H. Ocular Pharmacokinetics of a Novel Loteprednol Etabonate 0.4% Ophthalmic Formulation. Opthalmol Ther 3, 9 (2013).
34 Webre, J. M. et al. Rabbit and mouse models of HSV-1 latency, reactivation, and recurrent eye diseases. Journal of biomedicine & biotechnology 2012, 612316, doi:10.1155/2012/612316 (2012).
35 Hendricks, R. L. An immunologist's view of herpes simplex keratitis: Thygeson Lecture 1996, presented at the Ocular Microbiology and Immunology Group meeting, Oct. 26, 1996. Cornea 16, 503-506 (1997).
36 Rajasagi, N. K., Reddy, P. B., Mulik, S., Gjorstrup, P. & Rouse, B. T. Neuroprotectin D1 reduces the severity of herpes simplex virus-induced corneal immunopathology. Investigative ophthalmology & visual science 54, 6269-6279, doi:10.1167/iovs.13-12152 (2013).
37 Stuart, P. M. & Keadle, T. L. Recurrent herpetic stromal keratitis in mice: a model for studying human HSK. Clinical & developmental immunology 2012, 728480, doi: 10.1155/2012/728480 (2012).
38 Morris, J. et al. Recurrent herpetic stromal keratitis in mice, a model for studying human HSK. Journal of visualized experiments: JoVE, e4276, doi:10.3791/4276 (2012).

### Example 6

Introduction

Physiological angiogenesis and neovascularization are required for embryonic development¹, tissue remodeling and wound healing^{2,3}. However, in certain tissues and diseases, dysregulation of these tightly controlled processes can result in vascularization-mediated pathological conditions^{3,4,5}. Pathological vascularization and dysregulation of vascular function are critical determinates in the outcomes of many diseases, such as viral-mediated pathologies, cancer⁴, rheumatoid arthritis⁶, psoriasis⁷, and severe pulmonary infections^{8,9}. In addition, pathological vascularization within the eye, and especially within the normally avascular cornea, is the main contributor to many ocular diseases^{10,11}, including blinding stromal keratitis, proliferative retinopathies¹¹, and macular degeneration.

Recently, a new role in modulating inflammatory processes was discovered for the serotonin receptor family, also known as the 5-hydroxytyptamine receptors (5-HT)^{13,14}. Once thought to only be involved in modulating release of neurotransmitters in the central and peripheral nervous system, these GPCRs are finding new life as modulators of broad biological functions, including in cardiovascular biology and as immune regulators. It is becoming increasingly recognized that the 5-HT receptor family plays significant modulatory roles in many diseases, either promoting or suppressing disease progression through their activity. The finding that activation of the 5-HT2a receptor with the agonist DOI can effectively suppress inflammation by inhibiting TNFα activity^{15,16,17} opens the door to its potential therapeutic activity in treatment of many of the herein indicated diseases. Without wishing to be bound by theory, dysregulation of 5-HT receptor activation broadly influences the pathological outcomes of several vascularization- and inflammation-associated diseases, and that modulation of 5-HT receptor function can be utilized therapeutically to prevent and/or resolve the disease promoting sequelae. Studies described herein will determine the therapeutic viability and effectiveness of 5-HT2a agonists in resolving inflammation- and vascularization-associated disease processes of the eye by accomplishing the following objectives:

(I) To determine the ocular toxicity, safety, and tolerance of 5-HT2a agonist therapeutic formulations.

(II) To evaluate delivery, dosing and distribution of therapeutic formulations of 5-HT2a agonists.

(III) To evaluate the therapeutic efficacy of 5-HT2a agonists for amelioration of virus-associated diseases.

(IV) To evaluate the anti-inflammation and anti-neovascularization activities of 5-HT2a receptor agonists.

Pathological vascularization and dysregulation of vascular function are main contributors to all infectious and many non-infectious disease processes of the eye, lungs, and skin. In addition, vascularization of cancerous cells is essential for tumor growth and metastasis. Although there is precedence in the literature for contributions of serotonin/5-HT and 5-HT receptors to some of these processes, for the most part their contributions to disease development and resolution in the areas herein has not been explored. The new association of drug targetable 5-HT processes for these diseases represents a new and innovative approach. Pharmaceutical products that can ameliorate inflammation- and vascularization-associated disease manifestations represent an extremely sought after market sector with broad applicability for numerous indications. The included studies provide the necessary prerequisite and foundational information for expanding into these broad markets.

(I) To Determine the Ocular Toxicity, Safety, and Tolerance of 5-HT2a Agonist Therapeutic Formulations.

The establishment of a drug's toxicity, safety and tolerance profiles is a compulsory prerequisite to all subsequent efficacy trials. These profiles dictate a drug's practical concentrations, therapeutic indexes, and properties they impart to carrier formulations that may alter tolerability (ie. pH). In the exposed epithelium of the eye, which has regenerative and wound healing capacity that is critical for proper eye function, additional criteria must be met. A drug formulation must not: 1) Exhibit cellular toxicity to the corneal epithelium; 2) diminish cellular metabolic activity; 3) alter ocular physiological pH, which can burn the cornea; 4) inhibit replicative capacity of stem-like cells from the corneal limbus; 5) impair epithelial migration/wound healing. This objective will establish these foundational criteria and the functional parameters that can be employed within all subsequent in vitro and in vivo studies. It will also serve as the evaluation criteria for the institutional animal use panels.

This objective will provide foundational assessment of toxicity, safety, and tolerance profiles of 5-HT2a agonists 2 complementary Sub-Objectives:

Sub-Objective 1A:

To establish prerequisite ocular cytotoxicity and effects on ability to repair wounds for 5-HT2A agonist formulations. In vitro evaluation of toxicity will be demonstrated by evaluating cytotoxicity to corneal epithelium, scratch wound repair of corneal epithelium, and radial wound repair of corneal epithelium.

Direct Cytotoxicity/Safe Cellular Dosages: Assessment of drug and drug formulation cytotoxicity is critical prerequisite for determination of effective dosing range and subsequent therapeutic indexes. Given the nature of the eye, for topical ocular drops, cytotoxicity to corneal epithelium requires a multi-parameter assessment including, direct cellular toxicity, effects on wound healing and repair, and changes to cellular proliferation and metabolic energy production.

Pharmacological Cytotoxicity Assessments to Primary Human Corneal Epithelium:
1) Dose Dependent Cellular Cytotoxicity
2) Time-Dependent Cellular Cytotoxicity (daily and long-term assessments)
3) Determination of 50% Cellular Cytotoxicity (CC₅₀)

Cytotoxicity Assessments and Cell Viability will be Evaluated by:
1) Membrane Integrity Assays
2) Metabolic Activity Assays
3) Energy Production Assays
4) Cellular Proliferation Assays

Effects on Epithelial Wound Healing: The cornea necessarily has regenerative capacity that ensures maintenance of visual acuity. Damage to the corneal epithelium is repaired through a process of cellular replication and migration from the corneal limbus “fill in” sites of damage. Drugs that inhibit these processes are inherently toxic within the eye following short-term or long-term use. Therefore, assessment of the effects of 5-HT2a agonists in scratch (2 dimensional migration) and radial (proliferation and multidimensional migration) wound repair models at non-cytotoxic doses is an important safety analysis that dictates subsequent in vivo testing of the same parameters.

Assessments: Wound healing following scratch and radial de-epithelialization of a primary human corneal epithelial monolayer.

Wound Healing Assessments:
1) Concentration-Dependent Percent Healing in 24 hours
2) Kinetics of Wound Healing
3) Ocular Drug Carrier Effects on Wound Healing

Sub-Objective 1B: To evaluate in a rabbit eye model the in vivo toxicity and effects on wound repair for 5-HT2A agonist topical therapeutic formulations. In vivo evaluation of ocular toxicity comprises irritation/draize, scratch wound repair of corneal epithelium, and radial wound repair of corneal epithelium.

Daily clinical assessments comprise intraocular pressure, wound size, rate of closure, slit-lamp biomicroscopy, corneal neovascularization, corneal epithelium, corneal inflammation, epiphora, stromal inflammation, scleral inflammation, conjunctival inflammation, blepharitis, inflammatory discharge, behavioral toxicity.

Formulation of a topical ocular drug into an appropriate ophthalmic carrier solution can provide optimal delivery, distribution across the surface of the eye, and drug retention. However, inclusion of a test drug within these formulations can have undesirable outcomes, such as alteration of pH (even slightly basic solutions can burn cornea) or precipitation of compounds in solution. This objective will optimize the formulation of the 5-HT2a agonists in ophthalmic solutions by first assessing: short-term solubility and stability, pH, etc. These formulations will include maximal concentrations of drug that were determined to be non-toxic in the previous in vitro toxicity assessments (see sub-objective 1A). These formulations will subsequently be assessed in an escalating series of in vivo ocular toxicity models: 1) an ocular irritation model following repeated dosing; 2) a scratch wound healing model; 3) a radial wound healing model where >90% of the corneal epithelium will be removed and allowed to regenerate during repeated dosing. In addition, following completion of these studies, we will collect ocular tissues and blood samples for determination of drug distribution (see sub-objective 2A).

5HT2a Receptor Agonist Topical Ophthalmic Formulations:
1. Selection of topical ophthalmic carriers and non-toxic drug concentrations.
2. Determination of Solubility, pH, etc. Optimization of pH.
3. Assessment of short-term maintenance of formula properties: 8 days
4. Assessment of longer-term maintenance of formula properties: 30 days

Ocular Irritation Assessments Repeated Dosing (Rabbit Eye Model):
1. Short-term Acute Toxicity: 1 dose; 24 hour assessment of herein defined clinical parameters.
2. Repeated Dosing Toxicity: dosing 4-8× per day; Clinical Assessments 2× per day (morning/evening) as per herein defined clinical parameters. 7 days

Drug Effects on Ocular Wound Healing (Rabbit Eye Model):
1. Determination of Effects of Drug on Healing of Corneal Crosshatched Scratches
2. Determination of Effects of Drug on Healing of 10 mm Radial Corneal De-Epithelialization
3. Assessment of all clinical parameters defined herein daily

Although these studies are designed to assess ocular toxicity effects of 5HT2a receptor agonists ophthalmic formulations, they also will provide clinical information on reduction of surgical- or trauma-induced ocular neovascularization and inflammation, which worsens prognosis. Furthermore, effects of these drugs on intraocular pressure may give indications for use in diseases, such as glaucoma, or as an alternative for steroidal anti-inflammatories that increase IOP.

(II) To Evaluate Delivery, Dosing and Distribution of Therapeutic Formulations of 5-HT2a Agonists.

The tissue distribution and localized concentrations of 5-HT2a agonists following either ocular topical or systemic delivery can inform additional potential therapeutic indications.

This objective will be coordinated with the in vivo ocular safety and toxicity studies described in Sub-objective 1B. Following completion of all studies, eyes that were treated with drug concentrations that did not exhibit any ocular toxicity will be harvested and the following tissues collected: 1) Cornea; 2) Conjunctiva; 3) Sclera; 4) Aqueous Humor; 5) Vitreous Humor; 6) Retina; 7) Blood/Sera. Samples will be catalogued, and flash frozen and stored at −80C for future analysis.

A second evaluation of distribution following systemic delivery (IV administered through rabbit ear) will also be performed at 24 and 48 h to assess ocular distribution and concentrations following systemic administration.

(III) To Evaluate the Therapeutic Efficacy of 5-HT2a Agonists for Amelioration of Virus-Associated Diseases.

Infection- and inflammation-associated eye diseases are the leading causes of corneal blindness and visual morbidity, with over 500 million individuals affected⁹. Pathogen-associated ocular diseases are a complex combination of pathogen-mediated trauma and host-mediated pathologies, often with the most severe sequelae due to host inflammatory responses. Therefore, to prevent ocular disease development, an ideal drug will suppress both pathogen replication and host-mediated inflammatory responses. When available for ophthalmic use, anti-pathogen drugs can inhibit a pathogen's replication and often lessen the severity of pathogen-associated disease¹³. However, they can be specific to a given pathogen, elicit drug induced toxicity of the corneal epithelium¹⁴, and target only a single aspect of a pathogen's replication machinery. For persistent or recurrent ocular infections, such as HSV-1, long term use of these drugs can result in development of drug resistant variants. More importantly, current anti-pathogen drugs fail to inhibit host-mediated inflammatory and neovascularization responses and therefore, ocular disease can progress despite a drug's ability to control infection. Immunosuppressive drugs, such as dexamethasone, can control deleterious inflammation; however, they also license uncontrolled pathogen replication—a severe public health issue for pathogens, such as Adenovirus, which already causes epidemic outbreaks of keratoconjunctivitis. In addition, corticosteroid use is associated with loss of an intact corneal epithelial barrier, an increased risk of infection, increased ocular pressure and eventual deterioration of vision. This objective will be accomplished through 4 sub-objectives (A-D) that directly assess ocular and pulmonary indications for 5-HT2a agonists:

Sub-Objective 3A: To Evaluate the Therapeutic Efficacy of 5-HT2a Agonists in Resolution of Acute and Chronic Herpetic Keratitis.

Globally, infection- and inflammation-associated eye diseases are the leading causes of corneal blindness and visual morbidity, with over 500 million individuals affected¹⁸. The model ocular viral pathogen in these studies, Herpes Simplex virus type I (HSV-1), is present in 70-90% of the population and is the leading cause of corneal blindness in developed countries^{19,20}. The National Eye Institute estimates that 450,000 Americans have experienced some form of ocular herpetic disease, with 50,000 new and recurrent cases diagnosed¹⁹. Current anti-pathogen drugs fail to inhibit pathogen-induced inflammatory responses^{21,23}. As such, approximately 25% of cases present with serious inflammation-associated stromal keratitis. Individuals that have experienced ocular herpes, have a 50% chance of recurrence¹⁹. Each repeated episode triggers a chronic inflammatory disease process that that can result in vascularization and subsequent vision threatening scarring of the cornea that eventually requires corneal transplantation to resolve^{21,26}. Immuno-suppressive drugs, such as dexamethasone, can control deleterious inflammation; however, they also license uncontrolled pathogen replication and are associated with loss of an intact corneal epithelial barrier, increased ocular pressure and eventual deterioration of vision^{27,28}. By contrast, modulation of 5-HT receptor activity within the eye has been shown to decrease ophthalmic pressure²⁹. Combined with its newly discovered anti-inflammatory and anti-vascularization properties, its potential within the eye can be immense, for example, by replacing corticosteroids for several ocular disease indications.

Sub-objective 3A. 1 Rabbit Eye Model of Resolution and/or Prevention of Acute HSV Keratitis. The rabbit eye model of HSV-1 infection has been established as a gold-standard small-animal model assessment of a drug's ability to effect HSV-mediated acute ocular disease as its clinical and virological assessments closely mirror topical drug treatment effects in humans. Unlike the mouse eye, rabbit eye is in many respects more morphologically similar to the human eye and viral replication and disease course ensue in a manner similar to human disease. Table 7:

HSV-1 Screening in Rabbit Eyes
N = 8/group (16 eyes total) New Zealand Whites

| | |
|---|---|
| Group 1: | Vehicle Control |
| Group 2: | Test Drug, Dose 1 |
| Group 3: | Test Drug, Dose 2 |
| Group 4: | Reference Control 1% TFT (Viroptic) |
| | or 0.15% Ganciclovir Gel (Zirgan) |

New Zealand White rabbits (1.5-2 kgs) will have the corneas of both eyes scarified in a 4×4 cross hatched pattern and immediately inoculated with 3×10⁵ PFU of HSV-1 suspended in 50 μl of ophthalmic BSS. For prevention studies, animals will be randomly assigned into treatment groups and drugs or specific controls will be administered beginning at 3 hours post infection and clinical and virological assessments will commence beginning at 24 hours post infection. For resolution of infection and clinical disease, infection will proceed unabated for 3 days at which time animals will be clinically scored and accordingly sorted into clinically balanced groups prior to beginning treatment. This process normalizes inherent differences between animals and recapitulates the clinical scenario of a person reporting to the clinical with the onset of herpetic lesions. Topical drugs will be administered daily, such as 4-6× daily. As depicted herein, each morning til day 9 post infection scores for 9 clinical disease parameters (See FIG. 11, for example) will be assessed by slit lamp biomicroscopy. In addition, intraocular pressure will be assessed. Following scoring, infectious virus will be collected on ocular swabs from the tears in order to assess effects on viral replication without effecting clinical outcomes.

Effects on viral replication will be determined both by number of eyes positive for infectious virus, as well as the relative titer of virus per ocular swab for each day assessed as previously shown for Zirgan treatment in the figure to the right. Daily clinical disease assessments highlight the most pertinent disease sequelae observed in human infections. In addition to the clinical scoring of ocular disease, HSV replication in the eye can lead to neurological symptoms, encephalitis, and death. These parameters will be secondary endpoints within these studies.

Histological assessment of the eye will be performed to visualize what is scored clinically and virologically.

Table 8:

Relevance to Human Clinical Disease Outcomes: The rabbit eye model of acute HSV-1 infection closely mimics the virological, as well as the inflammation- and neovascularization-associated clinical parameters of a human infection. Most importantly, it

HSV-1 Screening in Mouse Eyes
N = 10/group (20 eyes total) BalbC or C57Bl

| | |
|---|---|
| Group 1: | Vehicle Control |
| Group 2: | Test Drug, Dose 1 |
| Group 3: | Test Drug, Dose 2 |
| Group 4: | Topical Reference Control 1% TFT (Viroptic) |
| | Systemic Reference Control Valacyclovir |

was the model that was utilized for development of the first ophthalmic anti-herpetics and has been shown to robustly predict pharmaceutical efficacy in the acute herpetic eye disease model.

Sub-objective 3A.2 Mouse Eye Model of Acute HSV Keratitis. Although the rabbit eye model is an ocular model for analysis of drug activity, the mouse eye model described herein is a cost effective and efficient means to screen active drug compounds and potential dosing strategies in order to determine potential dosing regimens and levels. In addition, this model permits assessment of the levels of HSV-1 that establishes latency within the trigeminal ganglia following treatment.

9 week old mice (approximately 18 gms) will have the corneas of both eyes scarified in a 4×4 cross hatched pattern and immediately inoculated with 1×10⁵ PFU of HSV-1 suspended in 5 μl of ophthalmic BSS. Animals will be randomly assigned into treatment groups and drugs or specific controls will be administered beginning at 3 hours post infection and clinical and virological assessments will commence beginning at 24 hours post infection. Route of administration and dosing schedule will be determined with the Project Officer and Sponsor. Topical drugs will be administered daily, such as 4-6× daily. Each morning til day 9 post infection scores for 9 clinical disease parameters will be assessed (as described on right) by slit lamp biomicroscopy and the weight of each animal will be determined. Following scoring, infectious virus will be collected daily on ocular swabs from the tears in order to assess effects on viral replication without effecting clinical outcomes.

Daily clinical disease assessments highlight the most pertinent disease sequelae observed in human infections. In addition to the clinical scoring of ocular disease, HSV replication in the eye can lead to neurological symptoms, encephalitis, and death. These parameters will be secondary endpoints within these studies.

For Analysis of Reduction of HSV-1 Latent within Neurons, the number of neurons and the levels of viral genomes latent within neurons can indicate the likelihood for increased episodes of reactivation and/or viral shedding. To determine the effects of treatment on the levels of HSV-1 viral genomes present within neurons following acute infection, virus will be allowed to establish latency for at least 30 days prior to any assessments and latency will be defined by 2 consecutive negative ocular swabs 30 days post infection. Trigeminal ganglia will be removed and the levels of viral genomes per TG will be determined by quantitative RT PCR relative to a standard curve. In addition, the ability of 5-HT2a agonists to inhibit ex vivo reactivation of HSV from latent neurons will be assessed, as this is the holy grail of current anti-herpetic drug activities and we have some indications that 5-HT2a agonists ameliorate the ability of HSV to reactivate from latently infected neurons.

Relevance to Human Clinical Disease Outcomes: Although the mouse eye differs structurally from the human eye, the mouse model is a well-established model for assessment of HSV-1 virological and host-mediated inflammation-associated disease processes. The main disease sequelae assessed here are also observed in human ocular disease. There are some differences in the timing and pathology of the disease outcomes, but overall the mouse ocular model is effective and efficient for screening and establishing drug responses and dosing regimens. In addition, the mouse model systems permit immunological analysis due to readily available immunological reagents and protocols.

Sub-objective 3.A3 Mouse Eye Model of Chronic HSV Stromal Keratitis. HSV-1 infections of the eye are the leading cause of infectious corneal blindness in the developed world. The disease course is due to both viral and host-mediated processes that are not always effectively controlled by ophthalmic antivirals. Although the acute rabbit eye model effectively assesses virological, clinical and pharmacological parameters of drug studies, the model does not efficiently permit assessment of contributing host-mediated disease factors that contribute to reproducible herpetic stromal keratitis. Infection of BalbC mice with HSV-1 RE results in nearly 100% of animals developing blinding stromal keratitis, with a large percentage still developing disease despite effective suppression of viral replication by the antiviral 1% TFT. A mouse model of HSV RE strain-induced stromal keratitis that has characteristics of chronic herpetic eye disease will be used.

TABLE 9
HSV-1 Screening in Mouse Eyes
N = 20/group starting (26 eyes total at study) BalbC

| | |
|---|---|
| Group 1: | Vehicle Control |
| Group 2: | Test Drug |
| Group 3: | Topical Reference Control 1% TFT (Viroptic) |
| | Systemic Reference Control Valacyclovir |

9 week old BalbC mice (approximately 18 gms) will have the corneas of both eyes scarified in a 4×4 cross hatched pattern and immediately inoculated with 5×10³ PFU of HSV-1 suspended in 5 μl of ophthalmic BSS. Animals will be randomly assigned into treatment groups and drugs or specific controls will be administered beginning at 3 hours post infection and clinical and virological assessments will commence daily beginning at 24 hours post infection until day 10. Topical drugs will be administered daily, such as 4-6× daily. At approximately 8-9 days post infection viral titers reach near undetectable in surviving animals and the host-mediated disease processes begin to commence. After the initial 10 days, clinical and virological assessments will continue every other morning til day 20 post infection scoring the 9 clinical disease parameters (described herein in acute disease model) by slit lamp biomicroscopy.

Although this model does assess virological outcomes during the acute phase of disease, the model assessments and endpoints largely look at clinical, immunological and histological disease outcomes. At peak disease end of study, animals will be sacrificed and the eyes removed for histological examination. Infiltration of immune cells, vascularization, thickening of stroma and epithelium, as well as fibrotic scarring will be examined.

Relevance to Human Clinical Disease Outcomes: Long-term chronic virus-initiated host-mediated disease processes are a hallmark of ocular HSV-1 infections within the human eye that can result in vision-threatening disease. The HSV-1 RE mouse model has many clinical characteristics of human disease processes that can be efficiently reproduced. Ophthalmic antiviral drugs that can affect these long-term disease processes, while also inhibiting viral replication are highly valued and this model gives an efficient cost effective means of assessing these therapeutic effects.

Sub-Objective 3B. Pilot Study- to Evaluate the Therapeutic Efficacy of 5-HT2a Agonists in Resolution of Acute Adenoviral Conjunctivitis (Pink-Eye).

There are two predominantly accepted ocular models of Adenovirus-associated eye disease-cotton rats and New Zealand White Rabbits. The rabbit eye model of Adenoviral replication and induction of associated disease will be used as the rabbit eye is a good predictor of ophthalmic drug efficacy and the associated disease outcomes mimic that observed in a human infection where Adenoviral-associated “pink-eye” can seasonally reach epidemic levels with no FDA approved ophthalmic antiviral currently available.

TABLE 10
Adenovirus Screening in Rabbit Eyes
N = 8/group (16 eyes total) New Zealand Whites

| | |
|---|---|
| Group 1: | Vehicle Control |
| Group 2: | Test Drug, Dose 1 |
| Group 3: | Test Drug, Dose 2 |
| Group 4: | Antiviral Reference Control 0.5% Cidofovir |
| | or Disease Reference Control 0.1% Dexamethasone |

New Zealand White rabbits (1.5-2 kgs) will have the corneas of both eyes scarified in a 4×4 cross hatched pattern and immediately inoculated with 2×10⁶ PFU of Adenovirus suspended in a 50 μl drop of ophthalmic BSS. Animals will be randomly assigned into treatment groups and drugs or specific controls will be administered beginning at 3 hours post infection. Topical drugs will be administered daily, such as 4-6× daily, except that the reference control cidofovir will be administered twice daily due to toxicity. As depicted herein, each morning, scores for 9 clinical disease parameters (described to the right) will be assessed by slit lamp biomicroscopy. In addition, intraocular pressure will be assessed. Following scoring, infectious virus will be collected on ocular swabs and tittered on A549 cells in order to assess effects on viral replication.

Given the viral- and host-mediated complexities of Adenovirus-induced eye disease the endpoints of this model include daily assessments of drug effects on both viral replication and inflammation- and neovascularization-associated clinical disease. Effects on viral replication will be determined both by # of eyes positive for infectious virus, as well as the relative titer of virus per ocular swab. Histological assessment of the eye at day 8 will be performed to visualize what is scored clinically and virologically.

Relevance to Human Clinical Disease Outcomes: The rabbit eye model of Adenoviral infection is clinically similar to human disease in that both virus-mediated destruction of the eye and host-mediated inflammatory processes are visualized and assessed. Like human disease outcomes, the infection is self-limiting in nature and encompasses components of ocular neovascularization, inflammation of sclera, blepharitis, and corneal involvement. The antiviral reference control Cidofovir has strong anti-Adenoviral activity; however, ocular toxicity and virus-induced inflammation is observed after a few days of use. Dexamethasone effectively suppresses disease presentation, but results in increased Adenoviral replication (orders of magnitude beyond that of control treatments).

Sub-Objective 3C. To Evaluate the Therapeutic Efficacy of 5HT2a Agonists in RSV-Associated Induction of Asthma.

Respiratory syncytial virus (RSV) infections are a significant cause of morbidity and mortality. The WHO estimates the global burden of RSV disease at 64 million cases and 160,000 deaths annually. In the United States, RSV is responsible for approximately 120,000 infant hospitalizations annually with estimated yearly Health care costs at $365-585 million. RSV infections in infants cause bronchitis, wheeze, and cough and are highly associated with development of asthma. RSV-associated pulmonary disease and development of asthma is an immunopathological condition with inflammatory and vascular etiologies. There are currently no effective treatment regimens that prevent development of chronic RSV-associated pulmonary disease. Recent findings that 5-HT2a agonists can prevent and help resolve development of asthma in other model systems indicates that they may be effective in these indications and preclude children from needing to cope with the lifelong struggles of RSV-induced asthma.

Model: A preclinical mouse model of infantile RSV infection that predisposes mice to long-term lung dysfunction and causes development of airway hyperresponsiveness that lasts into adulthood, mimicking immunophysiological changes experienced by children.

Method of drug delivery: Systemic delivery by daily intradermal injection or inhaled delivery.

Clinical Disease Evaluation: Pulmonary function testing on secondary challenge will be evaluated.

Pathological Disease Evaluation: At defined time points (6 days post-secondary exposure), lungs will be removed, inflated, fixed, and processed for HE and mucous production by histopathology. Changes in immune cell numbers and cell types within the lung will be assessed from BALFs.

Sub-Objective 3D to Evaluate the Therapeutic Efficacy of 5HT2a Agonists in Influenza-Associated Pulmonary Inflammation and Distress.

Seasonal influenza outbreaks result in 3-5 million cases of severe illness and approximately 350,000 to 500,000 deaths annually. Death occurs mainly in the young, old, or those with other health problems, including diabetes, cardiovascular, and pulmonary disorders. However, the high genetic variability of influenza results in sporadic antigenic shifts that result in resistance to antivirals and epidemic life-threatening outbreaks, which initially cause the most severe disease in the fit and healthy. This is thought to be due in part to these individuals ability to robustly respond to the viral infection, inducing deleterious inflammatory processes that elicit acute lung injury, increased pulmonary microvascular permeability and respiratory failure. As is the case for herpetic eye infections, current antivirals do not prevent or resolve these disease-associated processes and patients succumb to disease even after resolution of the self-limiting viral infection. Data indicates that modulation of 5-HT receptor pathways can prevent and reverse inflammation-associated disease processes within the lung, providing a means to treat the life-threatening disease sequelae that claims so many lives annually.

To evaluate the effects of 5-HT receptor modulation on severe pulmonary influenza infection, an established mouse model of influenza-induced pulmonary disease, which we have employed previously for evaluation of therapeutics for companies, will be utilized as depicted in Figure to right.

Start of treatment: Clinically relevant Day 3 when first symptoms appear.

Method of drug delivery: Systemic delivery by daily intradermal injection or inhaled delivery.

Positive control drug: Oseltamivir.

Treatment arms: A. 5-HT2a agonist, DOI, B. 5-HT antagonist.

Clinical Disease Evaluation: Clinical Illness Scores will be evaluated daily as shown in Figure and will continue for 14 days.

Pathological Disease Evaluation: At time of death or at defined time points, lungs will be removed, inflated, fixed, and processed for HE histopathology.

Without wishing to be bound by theory, 5-HT receptor agonists will suppress influenza-induced pulmonary inflammation and disease. Preclinical data derived from standardized clinical illness scores will be correlated with corresponding lung histopathological data to obtain an overall effectiveness assessment. The histopathological data will also be correlated with lung cellularity measurements obtained from BALF/FACs measurements. One caveat to these studies will be determining optimal drug dosing and delivery, whether it be by intranasal (direct) or intradermal (systemic) and at what dose. Dosing will initially follow that already defined by Dr. Nichols to produce protective effects without any behavioral modifications. As an additional and alternative approach, we will measure pulmonary lung function.

(IV) To Validate the Anti-Inflammation and Anti-Neovascularization Activities of 5-HT2a Receptor Agonists.

This objective will provide data validating that 5-HT2a receptor agonists directly affect neovascular/angiogenic processes. This data will complement the in vivo data obtained within the other objectives and provide mechanistic insights into how these agonists may be exerting their activity. These results can inform additional indications.

This objective will be accomplished through 3 complementary Sub-objectives:

Sub-Objective 4A:

To assess the effects of 5-HT2A agonist formulations on expression of mediators of inflammation and neovascularization (for example VEGF; nitric oxide; inflammatory cytokine and chemokine arrays). For example, treatment with 5-HT2a receptor agonists may suppress expression of specific mediators of inflammation- and neovascularization.

Effects of 5-HT2a receptor agonists on production of mediators of vascularization- and inflammation will be assessed. Specifically, (1) inflammation- and vascularization-associated PCR arrays will be utilized to assess the relative transcriptional profiles of genes associated with these disease promoting pathological processes following treatment and stimulation with various inducers. These arrays include analysis of growth factors and their receptors, signaling pathways, cell cycle regulatory pathways, cytokines and chemokines, adhesion molecules, proteases, and matrix proteins. These arrays also provide statistical analysis of how 5-HT2a receptor agonists affect transcriptional expression of genes in these pathways; (2) multiplexed quantitative protein analysis of secreted proteins will be performed via Bioplex following treatment and stimulation by various inducers that are associated with disease progression or poor prognosis. This work may be coupled with in vivo studies to yield additional mechanistic information on the anti-inflammatory and anti-neovascularization activity; and (3) nitric oxide and/or other reactive oxygen species involved in inflammation- and dysregulated vascular processes will be assessed from treated and stimulated macrophage/dendritic cell lineages.

Sub-Objective 4B:

To identify 5-HT2A agonists as direct suppressors of neovascularization/angiogenesis within in vitro and ex vivo models of vaculogenesis.

For example, 5-HT2a receptor agonists may abrogate endothelial cell migration, vessel sprouting, tube formation and stabilization.

Effects on Endothelial Cell Migration will be assessed. Migration of vascular endothelium is essential for formation of new vasculature. The ability 5-HT2a receptor agonists to inhibit primary HUVEC & HMVEC migration will be assessed via scratch wound healing assays and transwell migration assays. Wound healing migration assay: Confluent monolayers of HUVEC or HMVEC cells will be treated with DOI and a scratch wound will be induced in a cross pattern using a pipette tip. Cells will be microscopically imaged in realtime every 30 mins for 24 h on a live cell imager. The % closure and kinetics of closure will be determined and the ability of cells to migrate, form podia and cell extensions will be assessed from videos. Transwell migration assay: Cells will be seeded into the upper chamber of a transwell with VEGF maintained in the lower chamber to facilitate a chemotactic gradient. Wells will either be treated with DOI or controls and 24 h later cells that have migrated through the transwell will be imaged and quantified.

Effects on Vessel Sprouting and Tube Formation will be assessed. The ability of DOI to directly affect vessel sprouting and tube formation will be assessed in a matrigel tube formation assay and an aortic ring sprout and vascularization assay. Matrigel containing DOI or controls will be solidified onto 48 well plates. Vascular endothelial cells form 3D vascular tubes when plated onto matrigel. 12 and 24 h post-seeding, cells will be imaged and the extent of vascular tube formation, tube thickness, and branch points will be quantified using WimTube/Wimasis image analysis package. For the aortic sprouting assay, a mouse aorta will be removed and cut into 1 mm sections. The aorta will be placed upon the initial matrigel layer and overlayed with additional matrigel either containing DOI or controls. Aortas will be imaged daily using a stereomicroscope and quantified for: 1) initiation of vessel sprouting; 2) length of sprouts; 3) number of sprouts; 4) number of branch points.

Effects on Destabilizing Pre-Formed Tube Structures will be assessed. In vivo studies demonstrate that vascularization of the sclera and cornea had already occurred prior to beginning treatment. Therefore, DOI may not only inhibit progression of vascularization, but may resolve regions of neovascularization as indicated by a marked lessening of branch structures and a thinning of size and density of vessels. This may be due to DOI's ability to destabilize endothelial cell attachments, branch structures and vascular smooth muscle cell stabilization of vessels. To assess the destabilization activity of DOI, aortic rings will be allowed to grow tube structures with multiple branch points (these rings can be derived from control rings herein). Rings and tube structures will be imaged and then treated in growth factor media containing DOI or controls. The effects of 5-HT2a receptor agonists on maintenance of the branch points and tube structures will be imaged daily over 7 days and changes in tube structures, tube length, and branch points will be determined quantitatively. On day 7 rings and their attached structures will be fixed and a final assessment of structural integrity/stability will be determined by staining with smooth muscle actin, DAPI, and extracellular markers. Tubes will be imaged by deconvolution fluorescent microscopy and analyzed for overall differences in integrity, branch point stabilization, length of vessels, and presence of SMA markers surrounding formed tubes.

Sub-Objective 4C:

To validate the direct therapeutic potential of 5-HT2a agonist formulations in suppressing neovascularization/angiogenesis in non-inflammatory VEGF-corneal implant and matrigel plug implant in vivo models.

The infectious and biochemical models described herein are utilized to determine if 5-HT2a receptor agonists can prevent and resolve pathological vascularization. However, the nature of these studies precludes assignment of direct anti-vascularization activity as they are complicated by DOI's ability to inhibit viral replication, as well as its anti-inflammatory properties. VEGF is involved in inducing pathologic angiogenesis and increased vascular permeability in several serious eye diseases and in cancer. It will therefore be determined if DOI can directly block VEGF-mediated neovascularization in 2 complementary and directly translatable model systems: 1) in a VEGF-mediated ocular vascularization model that has become a standard for evaluating a drug's anti-vascularization activity; 2) in a matrigel implant tumor vascularization model that will assess the ability of locally and systemically administered DOI delivery to block vascularization of a disease tissue.

Sub-objective 4.C. 1 will validate DOI as a suppressor of VEGF-mediated ocular neovascularization and its associated pathology following implant of a slow-release VEGF pellet within a rabbit corneal micropocket.

A rabbit corneal micropocket assay will be used to assess the ability of topically administered DOI to prevent VEGF-mediated corneal vascularization^{30,31}. VEGF or saline control slow release micropellets will be generated as described previously³⁰. A corneal micropocket will be created in each rabbit eye 3 mm from the corneal limbus and micropellets will be implanted. Starting the day after implant, paired OD and OS eyes will be treated 4× daily with either control (OD eyes) or DOI (OS eyes) drops, respectively. The utilization of sister eyes for topical drug evaluation controls for animal-to-animal variability. Eyes will be clinically assessed daily and imaged by slit lamp biomicroscopy as described in all other ocular studies herein.

Quantitative Assessment of the Effects of DOI on VEGF-mediated vascularization. The daily area of corneal neovascularization will be determined as described previously^{30,31} by measuring the vessel length (L) from the limbus; the number of clock hours (C) of limbus involved; and the radius of the cornea (r). The amount of vascularization present in each eye on each day will be calculated by the formula: A=C/12×3.1416 (r²−(r−L)².

Assessment of clinical parameters in ocular neovascularization model. VEGF-induced vascularization and vessel permeability can lead to corneal edema, inflammation, and ocular clouding. Therefore, a panel of clinical parameters (described herein) will be assessed daily by fluorescent slit-lamp biomicroscopy to ascertain the therapeutic effects of topical DOI treatment on vascularization-mediated eye disease.

Evaluation of DOI suppression of VEGF-induced vascular leakage/permeability. Eyes treated with DOI not only showed a resolution of vascularization, but clinical presentation of edema and chemosis were greatly reduced. VEGF is a known mediator of vascular permeability and we have observed in this model system that edema and chemosis is common, irrespective of the extent of vascularization induced. To ascertain the effects of DOI on VEGF-mediated vascular leakage, FITC dextran will be systemically delivered via the ear vein and its presence will be assessed in the cornea, conjunctiva and sclera by fluorescent biomicroscopy. If visual examination shows clear indication that DOI suppresses vascular leakage by the relative absence of FITC within ocular tissues, animals will be sacrificed, corneas removed, and the relative levels of FITC will be determined spectrophotometrically following tissue homogenization and digestion. In addition, representative eyes will be processed for histology for direct visualization of extent of ocular vascularization and presence of edema.

Sub-objective 4C.2 will validate DOI as a suppressor of angiogenesis and vascularization of a matrigel plug implant in a tumor vascularization model.

Most anti-vascularization therapies are initially being developed to inhibit vascularization of solid tumors. This subobjective will expand the evaluation of DOI's ability to suppress angiogenesis and pathological vascularization, while simultaneously evaluating its effectiveness at suppressing vascularization following local or systemic delivery. Matrigel will be infused with VEGF (and/or PDGF) and either combined with DOI or a control treatment. The resulting paired suspensions will be injected subcutaneously into each mouse flank and allowed to polymerize³³. 7-10 days post implant, mice will be sacrificed and the extent of vascularization into the matrigel will be assessed. In a parallel experiment, untreated growth factor infused matrigel will be implanted and either DOI or controls will be delivered systemically by subQ injection, every day in order to evaluate whether systemic delivery can suppress vascularization of the matrigel implant.

Quantitative and Visual Assessment of the Effects of DOI on Matrigel Vascularization. The ability of localized and systemic delivery of DOI to thwart growth factor-induced vascularization of the matrigel implant will be assessed by: 1) direct visualization and imaging of the blood content present between the two treatments (matrigel is clear if no vascularization); 2) quantifying the amount of hemoglobin present within the excised implants using Drabkins reagent³⁴; 3) immuno-histopathological examination of sections of the plug with CD34 staining of the vascular endothelium; 4) FITC-dextran injection into the tail vein followed by excision of the explant and confocal 3D imaging of vascularization and extent of branching³³.

### REFERENCES CITED IN THIS EXAMPLE

1. Risau W. Mechanisms of angiogenesis. Nature. 1997 Apr. 17; 386(6626):671-4. Review.
2. Greaves N S, Ashcroft K J, Baguneid M, Bayat A. Current understanding of molecular and cellular mechanisms in fibroplasia and angiogenesis during acute wound healing. J Dermatol Sci. 2013 December; 72(3):206-17. Epub 2013 Jul. 30. Review.
3. Carmeliet P. Angiogenesis in health and disease. Nat Med. 2003 June; 9(6):653-60. Review 4. Folkman J. Angiogenesis in cancer, vascular, rheumatoid and other disease. Nat Med. 1995 January; 1(1):27-31. Review.
5. Chung A S, Ferrara N. Developmental and pathological angiogenesis. Annu Rev Cell Dev Biol. 2011; 27:563-84. Epub 2011 Jul. 13. Review.
6. Khong T L, Larsen H, Raatz Y, Paleolog E. Angiogenesis as a therapeutic target in arthritis: learning the lessons of the colorectal cancer experience. Angiogenesis. 2007; 10(4):243-58. Epub 2007 Sep. 6.
7. Varricchi G, Granata F, Loffredo S, Genovese A, Marone G. Angiogenesis and lymphangiogenesis in inflammatory skin disorders. Am Acad Dermatol. 2015 July; 73(1):144-53. Epub 2015 Apr. 25. Review.
8. Sousa A, Len O, Escola-Verge L, Magnifico B, Mora C, Papiol E, Revilla E, Almirante B. Influenza A virus infection is associated with systemic capillary leak syndrome: case report and systematic review of the literature. Antivir Ther. 2016; 21(2):181-3. Epub 2015 Sep. 2.
9. Armstrong S M, Mubareka S, Lee W L. The lung microvascular endothelium as a therapeutic target in severe influenza. Antiviral Res. 2013 August; 99(2):113-8. Epub 2013 May 16. Review.
10. Bradley J, Ju M, Robinson G S. Combination therapy for the treatment of ocular neovascularization. Angiogenesis. 2007; 10(2):141-8. Epub 2007 Mar. 13. Review.
11. Chen J, Smith L E. Retinopathy of prematurity. Angiogenesis. 2007; 10(2):133-40. Epub 2007 Feb. 27. Review.
12. Li W, Man X Y, Chen J Q, Zhou J, Cai S Q, Zheng M. Targeting VEGF/VEGFR in the treatment of psoriasis. Discov Med. 2014 September; 18(98):97-104. Review.
13. Di Rosso M E, Palumbo M L, Genaro A M. Immunomodulatory effects of fluoxetine: A new potential pharmacological action for a classic antidepressant drug? Pharmacol Res. 2015 Nov. 28. pii: S1043-6618(15)₀₀₂₇₉-0. [Epub ahead of print] Review.
14. Worthington J J. The intestinal immunoendocrine axis: novel cross-talk between enteroendocrine cells and the immune system during infection and inflammatory disease. Biochem Soc Trans. 2015 August; 43(4):727-33. Epub 2015 Aug. 3. Review.
15. Nau F Jr, Yu B, Martin D, Nichols C D. Serotonin 5-HT2A receptor activation blocks TNF-α mediated inflammation in vivo. PLoS One. 2013 Oct. 2; 8(10):e75426. eCollection 2013.
16. Yu B, Becnel J, Zerfaoui M, Rohatgi R, Boulares A H, Nichols C D. Serotonin 5-hydroxytryptamine(2A) receptor activation suppresses tumor necrosis factor-alpha-induced inflammation with extraordinary potency. J Pharmacol Exp Ther. 2008 November; 327(2):316-23. Epub 2008 Aug. 15.
17. Nau F Jr, Miller J, Saravia J, Ahlert T, Yu B, Happel K I, Cormier S A, Nichols C D. Serotonin 5-HT₂ receptor activation prevents allergic asthma in a mouse model. Am J Physiol Lung Cell Mol Physiol. 2015 Jan. 15; 308(2):L191-8.
18. Whitcher J P, Srinivasan M, Upadhyay M P. Corneal blindness: a global perspective Bulletin of the World Health Organization, 2001, 79: 214-221.
19. Facts about the cornea and corneal disease. National Eye Institute website, March 2010. Available at http://www.nei.nih.gov/health/cornealdisease/
20. Liesegang T J. Herpes simplex virus epidemiology and ocular importance. Cornea. 2001 January; 20(1):1-13. Review.
21. Rowe A M, St Leger A J, Jeon S, Dhaliwal D K, Knickelbein J E, Hendricks R L. Herpes keratitis. Prog Retin Eye Res. 2013 January; 32:88-101.
22. Pavan-Langston, D., Foster, C. S., Trifluorothymidine and idoxuridine therapy of ocular herpes. Am. J. Ophthalmol. 1977. 84, 818-825.
23. Imperia P S, Lazarus H M, Dunkel E C, Pavan-Langston D, Geary P A, Lass J H. An in vitro study of ophthalmic antiviral agent toxicity on rabbit corneal epithelium. Antiviral Res. 1988 July; 9(4):263-72.
24. Choong K, Walker N J, Apel A J, Whitby M. Aciclovir-resistant herpes keratitis. Clin Experiment Ophthalmol. 2010 April; 38(3):309-13.
25. Piret J, Boivin G. Resistance of herpes simplex viruses to nucleoside analogues: mechanisms, prevalence, and management. Antimicrob Agents Chemother. 2011 February; 55(2):459-72.
26. Kaufman H E. Treatment of viral diseases of the cornea and external eye. Prog Retin Eye Res. 2000 January; 19(1):69-85.
27. Aquavella J V, Gasset A R, Dohlman C H. Corticosteroids in corneal wound healing. Am J Ophthalmol 1964; 58: 621-6.
28. Mitsui Y, Hanabusa J. Corneal infections after cortisone therapy. Br J Ophthalmol 1955; 39: 244-50.
29. Sharif N A. Serotonin-2 receptor agonists as novel ocular hypotensive agents and their cellular and molecular mechanisms of action. Curr Drug Targets. 2010 August; 11(8):978-93. Review.
30. Bhattacharjee P S, Huq T S, Mandal T K, Graves R A, Muniruzzaman S, Clement C, McFerrin H E, Hill J M. A novel peptide derived from human apolipoprotein E is an inhibitor of tumor growth and ocular angiogenesis. PLoS One. 2011 Jan. 6; 6(1):e15905.
31. D'Amato R J, Loughnan M S, Flynn E, Folkman J. Thalidomide is an inhibitor of angiogenesis. Proc Natl Acad Sci USA. 1994 Apr. 26; 91(9):4082-5.
32. Kim L A, D'Amore P A. A brief history of anti-VEGF for the treatment of ocular angiogenesis. Am J Pathol. 2012 August; 181(2):376-9. Epub 2012 Jun. 29. Review
33. Baker M, Robinson S D, Lechertier T, Barber P R, Tavora B, D'Amico G, Jones D T, Vojnovic B, Hodivala-Dilke K. Use of the mouse aortic ring assay to study angiogenesis. Nat Protoc. 2011 Dec. 22; 7(1):89-104.
34. Balasubramaniam P, Malathi A. Comparative study of hemoglobin estimated by Drabkin's and Sahli's methods. J Postgrad Med. 1992 January-March; 38(1):8-9.
35. Csonka C, Pali T, Bencsik P, Gorbe A, Ferdinandy P, Csont T. Measurement of nitric oxide in biological samples. Br J Pharmacol. 2014 Jul. 2. [Epub ahead of print]

### Example 7

Aortic Ring Assay Protocol

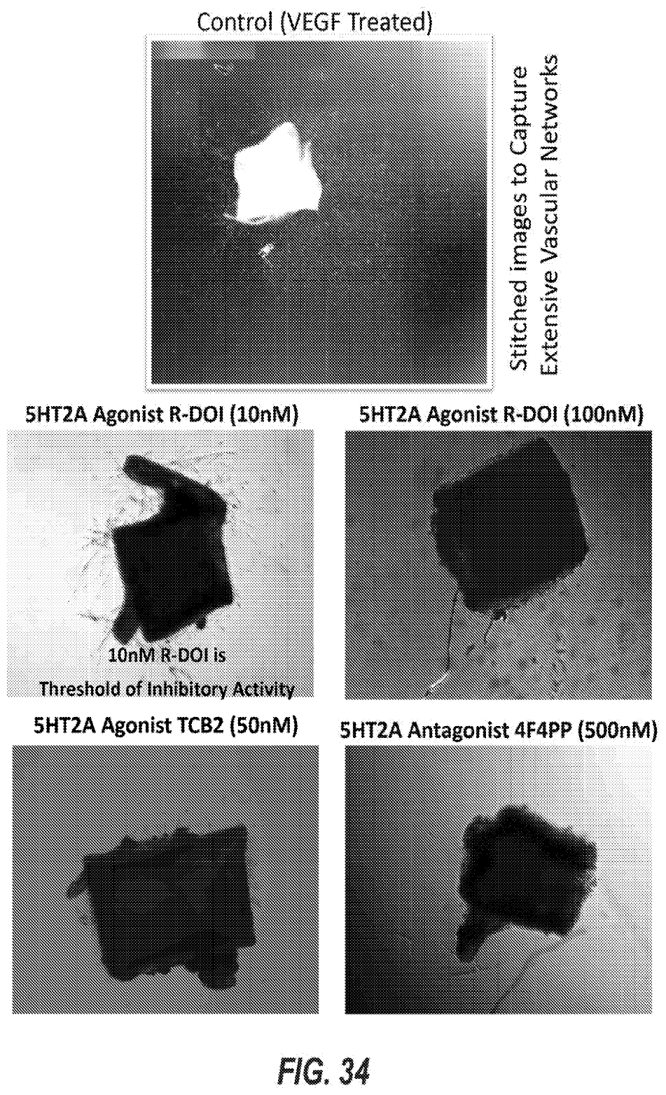

Referring to FIG. 34 for example, the aortas from sacrificed mice are removed, cleaned, and dissected into 1 mm tubule sections. These aortic rings are implanted into matrigel basement membrane and incubated in endothelial cell growth medium containing vascular endothelial growth factor. Aortic rings are continuously incubated in the presence of 5HT2A receptor agonists and antagonists at the indicated concentrations and examined by microscopy daily. Extensive sprouting, branching and networking of new blood vessels can be observed in control aortic rings, where multiple images needed to be stitched together in order to capture the extensive blood vessel network formed.

By contrast, 5HT2A agonists (R-DOI and TCB2), inhibited blood vessel sprouting, branching and formation. It was also determined that at a 10 nM concentration of R-DOI, the inhibitory effects for neovascularization, sprouting, branching, and networking began to no longer be as effective.

Unexpectedly, the 5HT2A antagonist (4F4PP) had similar neovascularization inhibitory effects as the agonists (R-DOI and TCB2).

Endothelial Tubule Formation Assay

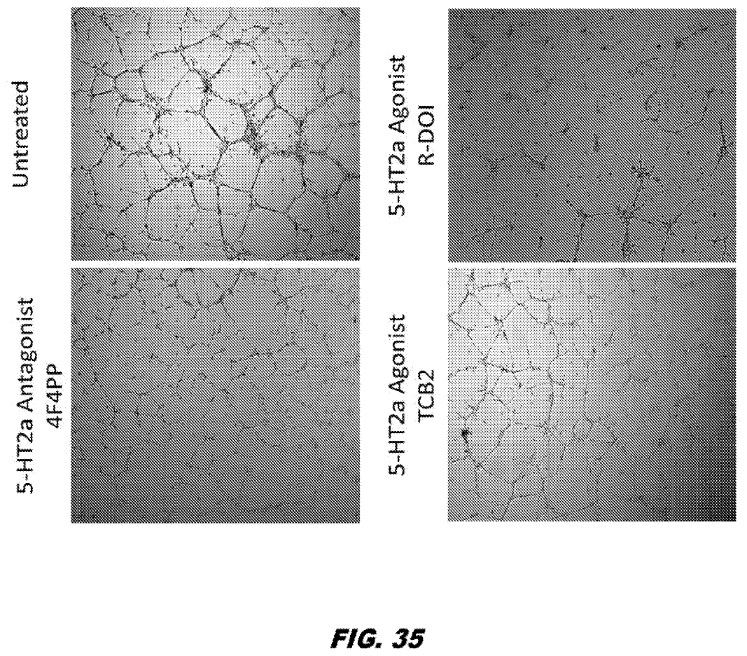

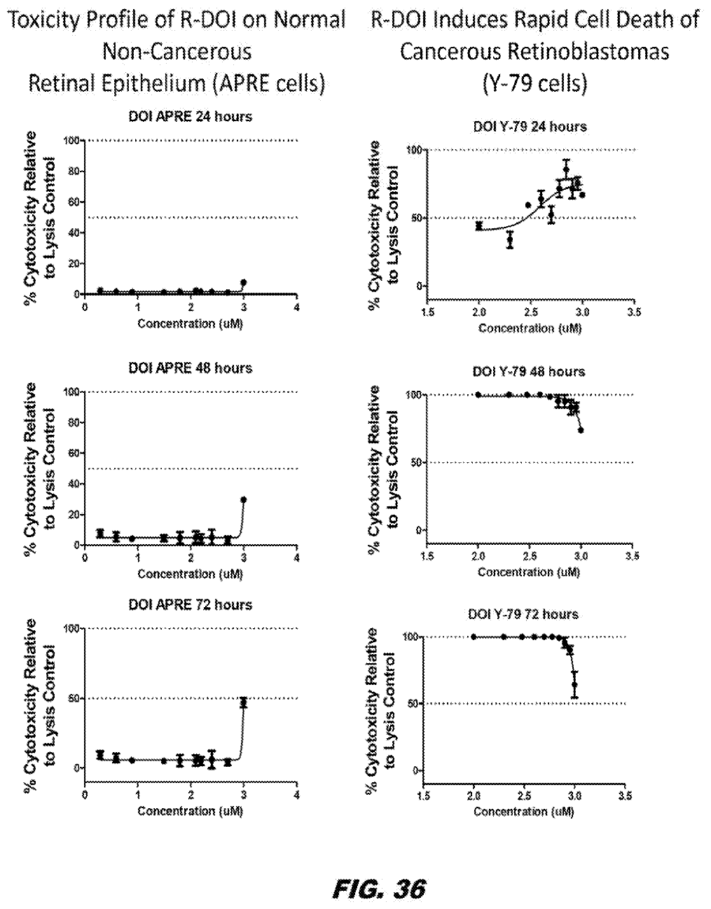

Referring to FIG. 35 for example, to assess the ability of 5-HT2A agonists and antagonists to inhibit capillary-like endothelial tube formation, human microvascular endothelial cells were seeded into geltrex basement membrane and overlaid with endothelial growth medium containing vascular endothelial growth factor (VEGF) and the indicated drugs. Both 5HT2A agonists and antagonists disrupted formation of endothelial tube networks, the formation of branching, complex capillary structures, and interconnectivity of capillary tubes.

### Example 8

Murine Model of HSV-1 (RE) Chronic Herpetic Keratitis.

BALB/c eyes were scarified and infected with 10,000 PFU per eye of HSV-1 RE, an HSV-1 strain that causes herpetic keratitis in 100% of ocular infections.

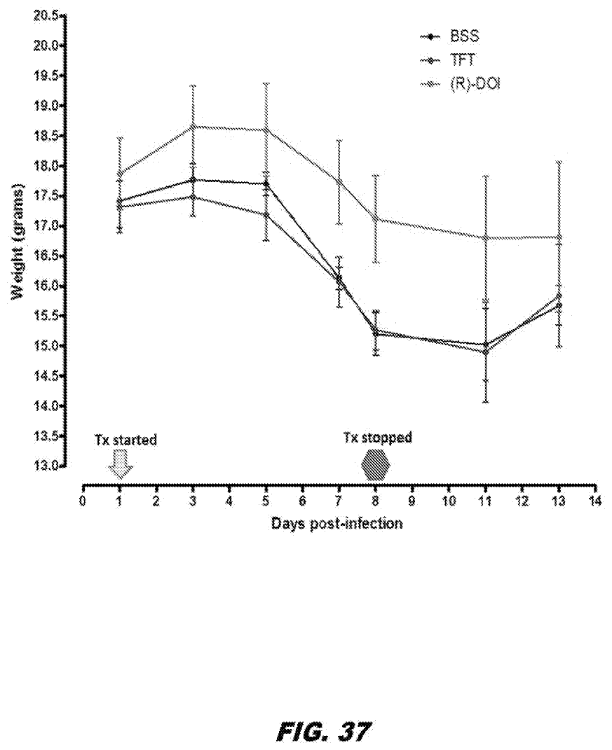

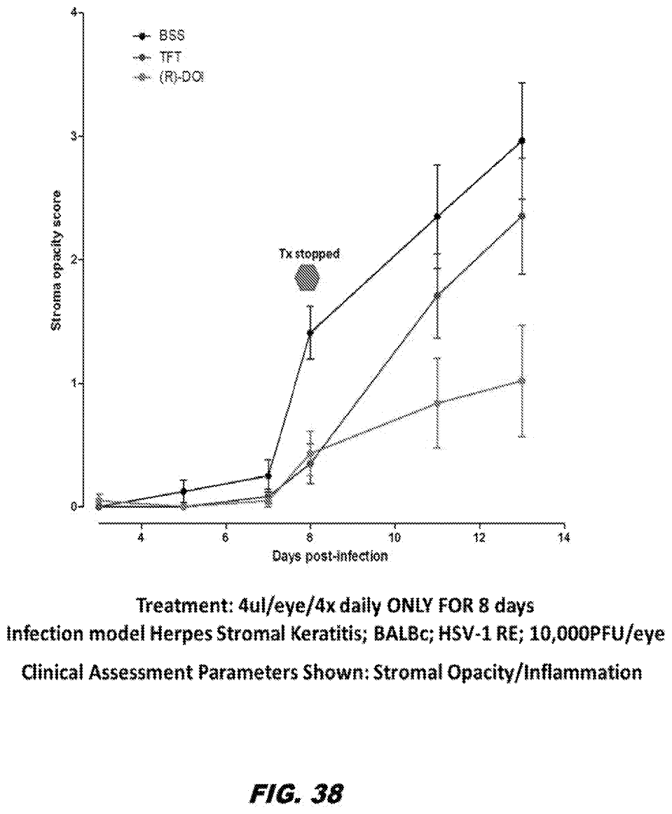

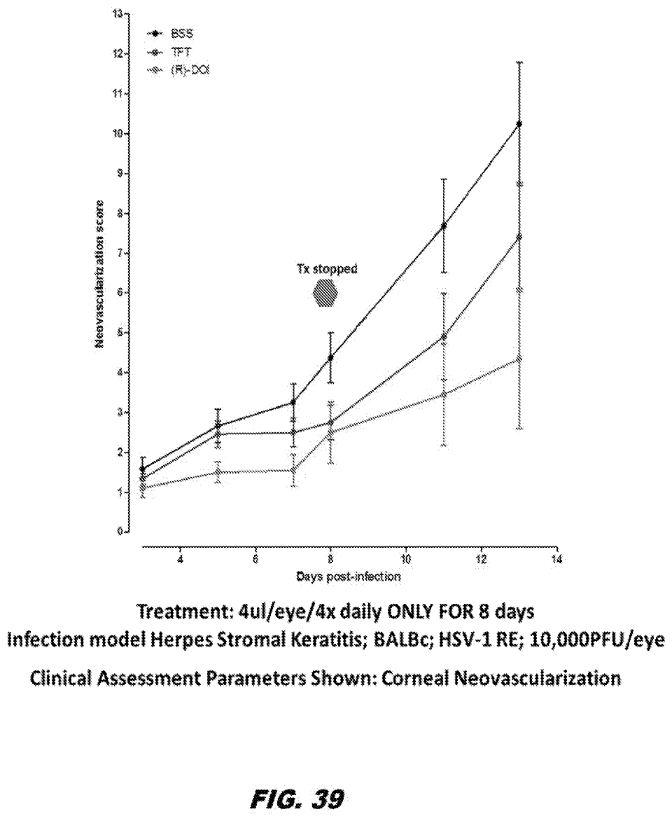

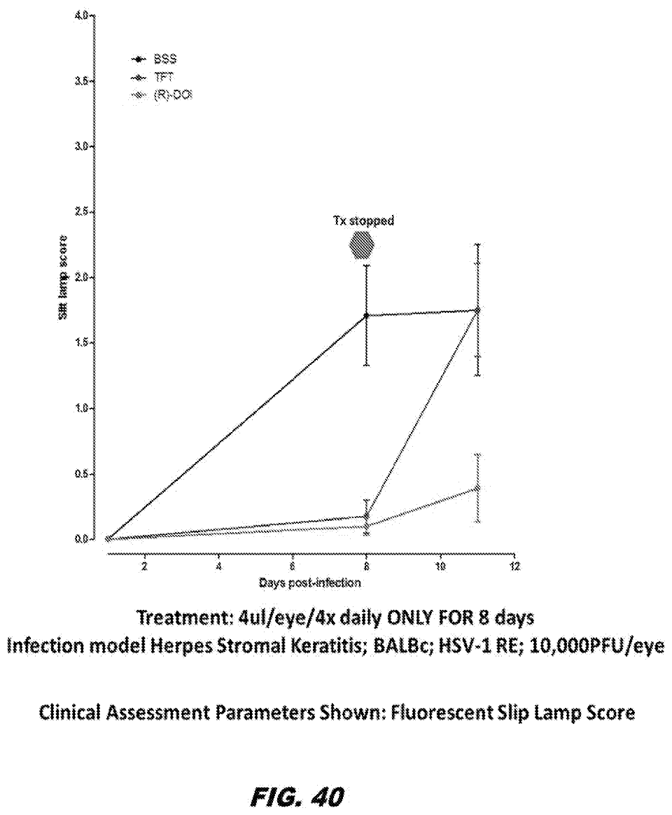

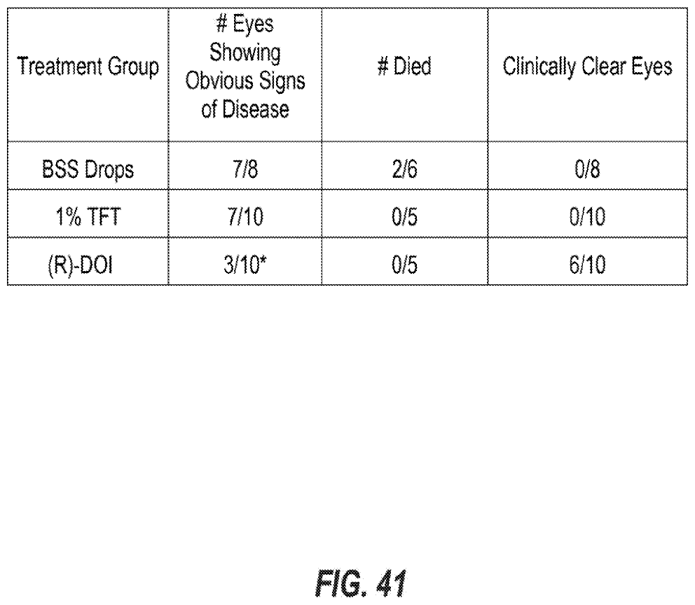

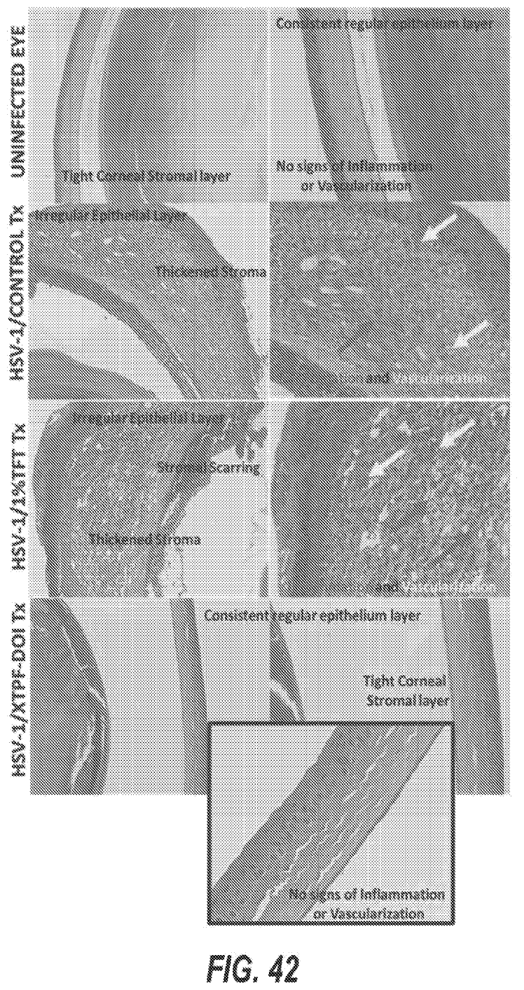

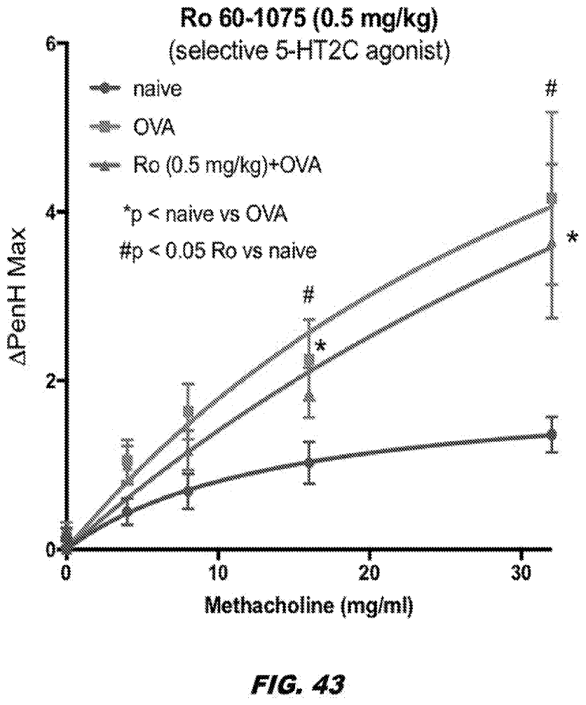

Referring to FIGS. 37-42, for example, individual clinical parameters were monitored and scored in a masked fashion for 15 days post infection. Parameters included weight, death, epiphora, blepharitis, corneal epithelium, corneal neovascularization, stromal opacity, scleral inflammation and fluorescently visualized slitlamp biomicroscopy.

24 hours post infection, topical drops were applied in a masked fashion 4× per day at 4 ul per drop to the surface of the eye. Topical drops consisted of 1% Trifluorthymidine (1% TFT; Viroptic), Ophthalmic Balanced Salt Solution (BSS), or (R)-DOI in Ophthalmic BSS.

16 BALB/c mice, which preferentially respond with a TH2 biased immune response, were randomly sorted into 3 treatment arms: 1) Ophthalmic Balanced Saline Solution (BSS) treated (6 mice); 2) DOI treated ((R)-DOI) (5 mice); 3) 1.0% TFT (5 mice). Animals were anesthetized with xylene:ketamine and both eyes were scarified in a cross hatch pattern using a curved needle. Immediately following ocular scarification, eyes were inoculated with a 3 microliter drop containing 10,000 plaque forming units (PFU) of Herpes Simplex Virus type 1 (HSV-1) RE strain. The next morning following infection animals were treated with the respective treatment as assigned within their treatment arm. Treatments were applied topically to the eye in a 4 microliter drop. Drops were applied 4× daily from 9 am to 5:30 pm starting immediately following clinical scoring. Treatments were applied for the first 8 days post infection and then stopped on day 8. Clinical scoring was done using a slit lamp biomicroscope magnified at 16× on the days indicated by a single individual masked to the drug treatment parameters. Slit lamp biomicroscopy also included fluorescein exclusion labeling of the corneal surface following scoring of all clinical parameters. Each eye was scored independently. Animal deaths were recorded if euthanasia was required due to severe encephalitis or if animals died from HSV-associated disease. Clinically clear eyes were scored as such if no apparent signs of disease were present in any clinical parameter during the chronic phase.

At day 15 post infection, a stage that would be during chronic immune-associated disease with no virus present, animals were euthanized and the eyes were removed for histology. Random representative eyes were prepared by taking sections through the central cornea and processed by H&E histology for visualization. Sections were examined microscopically and photographed across the central cornea. Multiple eyes from each group that showed the best representation of that groups clinical scores extremes and midpoints are shown.

The 5HT Agonist, DOI, Inhibits HSV-1 Neuronal Reactivation from Latency within Trigeminal Ganglia.

14 trigeminal ganglia from 7 ocularly infected mice that contained latent HSV-1 genomes within its neurons for greater than 60 days were removed, randomly divided into 2 groups of 7 ganglia, and were subsequently explanted and eviscerated in media that contained either 500 nM of DOI or an equivalent buffer control without drug. HSV-1 reactivation from latent neurons was induced using hyperthermic shock (42C) for 1 hour. Each day for 10 days post explant and induction of reactivation, ⅕ volume of media volume was removed and assessed for the presence of infectious HSV-1, indicating reactivation of virus from latency. This volume was replaced with media that contained either 500 nM of DOI drug or an equivalent of mock carrier buffer.

The 5HT agonist, DOI, maintains latency of HSV-1 within reactivation induced neurons as observed by the number and percentage of neurons positive for the presence of any infectious HSV-1. In addition, there was a significant delay in reactivation (2 fold greater) of HSV-1 from TGs that showed slight positivity for eventual presence of infectious virus.

In addition, the 5HT agonist, DOI significantly inhibited the degree of reactivation and amount of infectious virus shed from latent neurons. Analysis of average total reactivated infectious virus (PFU/ml/TG) or total reactivated infectious virus per positive TG (PFU/ml/positive TG) both indicate that DOI suppressed HSV reactivation, active replication, and shedding of infectious virus from latent neurons relative to mock treated neurons.

TABLE 11

| | Days post Explant | | | | | | | | | |
|---|---|---|---|---|---|---|---|---|---|---|
| | 1 | 2 | 3 | 4 | 5 | 6 | 7 | 8 | 9 | 10 |
| DOI 500 nM | 0/7 (0%) | 0/7 (0%) | 0/7 (0%) | 0/7 (0%) | 0/7 (0%) | 0/7 (0%) | 0/7 (0%) | 1/7 (14.3%) | 1/7 (14.3%) | 2/7 (28.6%) |
| Mock | 0/7 (0%) | 0/7 (0%) | 0/7 (0%) | 0/7 (0%) | 2/7 (28.6%) | 4/7 (57.1%) | 4/7 (57.1%) | 5/7 (71.4%) | 5/7 (71.4%) | 5/7 (71.4%) |

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method of reducing or ameliorating vascularization-associated pathology in a non-ocular tissue of a subject, the method comprising administering to the subject afflicted with a disease associated with tissue vascularization-associated pathology a therapeutically effective amount of a composition comprising a serotonin receptor agonist.

2. The method of claim 1, wherein the tissue comprises an immunologically-restricted tissue.

3. A method of reducing or ameliorating a hypersensitivity or a hypersensitivitiy-associated disease process in an immunologically-restricted tissue of a subject, the method comprising administering to a subject afflicted with a hypersensitivity or a hypersensitivity-associated disease process in an immunologically-restricted tissue a therapeutically effective amount of a composition comprising a serotonin receptor agonist.

4. A method of treating a vascularization-associated non-ocular disease in a subject, the method comprising administering to a subject afflicted with a vascularization-associated disease a therapeutically effective amount of a composition comprising a serotonin receptor agonist.

5. A method of treating a hypersensitivity-associated ocular disease in a subject, the method comprising administering to a subject afflicted with a hypersensitivity-associated ocular disease a therapeutically effective amount of a composition comprising a serotonin receptor agonist.

6. The method of claim 2, wherein the immunologically-restricted tissue comprises a tissue of the lung, skin, brain, or a combination thereof.

7. The method of claim 2 or 3, wherein an immunologically-restricted tissue is infected.

8. The method of claim 7, wherein the infection comprises a viral infection, a bacterial infection, a fungal infection, a protozoan infection, or a combination thereof.

9. The method of claim 7 or 38, wherein a DNA virus causes infection.

10. The method of claim 7 or 38, wherein a RNA virus causes infection.

11. The method of claim 1, 3, 4, 5, or 38 wherein the serotonin receptor agonist comprises a compound of formula (I)

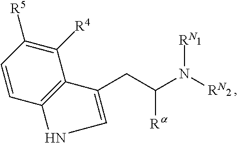

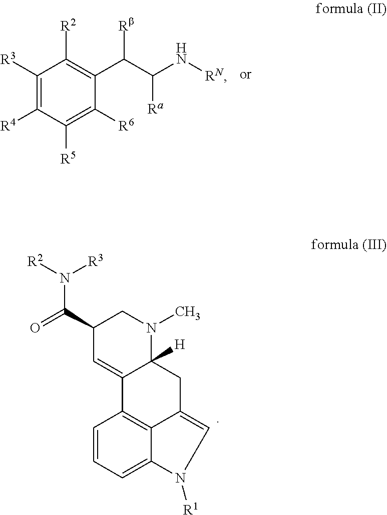

12. The method of claim 1, 3, 4, 5, or 38 wherein the serotonin receptor agonist comprises 2,5-Dimethoxy-4-iodoamphetamine (DOI).

13. The method of claim 1, 3, 4, 5, or 38 wherein the method comprises a low dose of the serotonin receptor agonist.

14. The method of claim 1, 3, 4, 5, or 38 wherein the composition further comprises at least one antimicrobial agent, at least one anti-pathogenic agent, at least one drug, or a combination thereof.

15. The method of claim 14, wherein the antimicrobial agent comprises an antiviral agent, an antibacterial agent, an antifungal agent, an antiprotozoal agent, or a combination thereof.

16. The method of claim 1, 3, 4, 5, or 38, wherein the serotonin receptor comprises the 5-HT2A serotonin receptor.

17. The method of claim 7, wherein the infection causes pathogenesis in at least one tissue of the subject.

18. The method of claim 17, wherein the pathogenesis comprises angiogenesis, neovascularization, hypersensitivity, vascular leakage, vascular permeability, edema, lymphangiogenesis, hypertension, or a combination thereof.

19. The method of claim 17, wherein the pathogenesis affects a tissue of the eye, lung, skin, brain, or a combination thereof.

20. The method of claim 1 or 4, wherein vascularization-associated pathologies comprises angiogenesis of blood vessels, angiogenesis of lymphatic vessels, vascular leakage, vascular permeability, vasoconstriction, vasodialation, vascular occlusions, hypertension, edema, ischemia, or a combination thereof.

21. A composition comprising at least one serotonin receptor agonist and at least one antimicrobial agent selected from an antibacterial agent, an antifungal agent, and an antiprotozoal agent.

22. The method of claim 21, wherein the composition further comprises at least one antiviral agent.

23. The composition of claim 21, wherein the composition further comprises at least one antipathogenic agent.

24. The composition of claim 21, wherein the composition comprises a low-dose of the serotonin receptor agonist.

25. The composition of claim 21, wherein the serotonin receptor agonist comprises a compound of

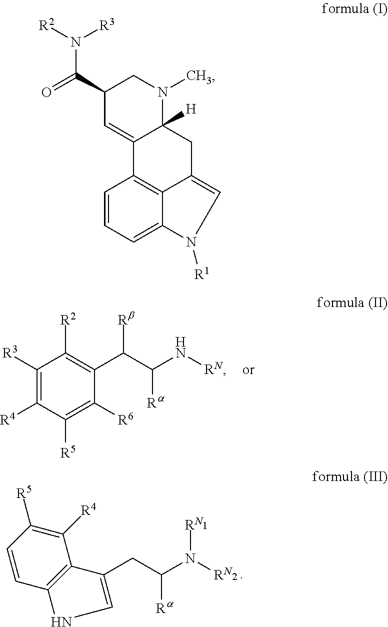

26. The composition of claim 21, wherein the serotonin receptor agonist comprises 2,5-Dimethoxy-4-iodoamphetamine (DOI).

27. The composition of claim 21, wherein the composition comprises an ocular drop, dermal patch, ocular gel, topical gel, systemic delivery system, enteric capsule, nebulized inhalant, inhalant, intrathecal composition, or an injectable.

28. The composition of claim 21, wherein the serotonin receptor comprises the 5-HT2A serotonin receptor.

29. The composition of claim 21, wherein the serotonin receptor agonist comprises a 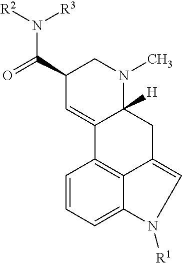
chemical having the following formula:
wherein R¹, R², and R³ are selected from the group comprising CH₂CH₃, CH(CH₃)CH₂CH₃, CH(CH₃)CH₂CH₂CH₃, C₂H₅, CH₂CH₂CH₃, CH(CH₃)₂ and H.

30. The composition of claim 21, wherein the serotonin receptor agonist comprises a 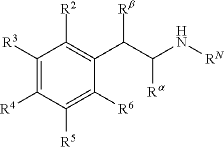
chemical having the following formula:
wherein R^{α}, R^{β}, R², R³, R⁴, R⁵, R⁶ and R^{N} are selected from the group comprising OCH₃, CH₃, SCH₃, Br, I, CH₂CH(CH₃)₂, and H.

31. The composition of claim 21, wherein the serotonin receptor agonist comprises a chemical having the following formula: 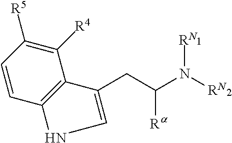
wherein R^{α}, R^{N}₁, R^{N}₂, R⁴ and R⁵ are selected from the group comprising C, CH₃, OH, F, OCH₃ and H.

32. The method of claim 3, wherein the immunologically-restricted tissue comprises a tissue of the lung, skin, brain, eye or a combination thereof.

33. The method of claim 1, 17, and 20, wherein the pathogenesis comprises hypersensitivity, a hypersensitivity-associated disease process, vascularization, vascular leakage, vascular permeability, angiogenesis, lymphangiogenesis, neovascularization, vasodialation, vasoconstriction, vascular occlusions, edema, corneal epithelial defects, increased intraocular pressure, increased oxygen saturation, ischemia, haemorrhage, necrotizing inflammation, epithelial hyperproliferation, epithelial thickening, fibrosis, or a combination thereof.

34. The method of claim 7, wherein the infection comprises a viral infection.

35. The method of claim 33, wherein the viral infection comprises herpetic keratitis, stromal keratitis, herpetic uveitis, herpetic iritis, viral keratoconjunctivitis, viral retinitis, adenoviral conjunctivitis.

36. The method of claim 5 wherein the ocular disease comprises AMD, choroidal vascularization, diabetic retinopathies, viral retinopathies, glaucoma, corneal allograft transplant rejection, ocular hypertension, corneal neovascularization, keratoconjunctivitis, viral conjunctivitis, allergic conjunctivitis, uveitis, iritis, or keratitis.

37. The method of claim 33, wherein the viral infection is associated with ulceration, keratoconjunctivitis, blepharitis, neovascularization, edema, endophthalmitis, haemorrhage, photophobia, glaucoma, necrotizing inflammation, loss of vision, reduced vision, uveitis, iritis, ocular redness, scleral injection, retinitis, fibrosis, epithelial thickening, blepharitis, endophthalmitis, photophobia, glaucoma, loss of vision, or a combination thereof.

38. A method of delaying or preventing viral reactivation in a tissue of a subject, the method comprising administering to the subject afflicted with a persistent viral infection a therapeutically effective amount of a composition comprising a serotonin receptor agonist.
